(19) United States
(12) Patent Application Publication
(10) Pub. No.: US 2021/0158906 A1
(43) Pub. Date: May 27, 2021

(54) SYSTEMS, METHODS AND PROCESSES FOR DYNAMIC DATA MONITORING AND REAL-TIME OPTIMIZATION OF ONGOING CLINICAL RESEARCH TRIALS

(71) Applicant: BRIGHT CLINICAL RESEARCH LIMITED, Hong Kong (CN)

(72) Inventors: Tailiang XIE, Belle Mead, NJ (US); Ping Gao, Bridgewater, NJ (US)

(21) Appl. No.: 17/165,022

(22) Filed: Feb. 2, 2021

Related U.S. Application Data
(63) Continuation-in-part of application No. PCT/IB2019/056613, filed on Aug. 2, 2019.
(60) Provisional application No. 62/807,584, filed on Feb. 19, 2019.
(60) Provisional application No. 62/713,565, filed on Aug. 2, 2018.

(51) Int. Cl. G16H 10/20; G06F 17/18; G06F 3/0482

(52) U.S. Cl. CPC G16H 10/20; G06F 3/0482; G06F 17/18

(57) ABSTRACT

This invention relates to a method and process which dynamically monitors data from an on-going randomized clinical trial associated with a drug, device, or treatment. In one embodiment, the present invention automatically and continuously unblinds the study data without human involvement. In one embodiment, a complete trace of statistical parameters such as treatment effect, trend ratio, maximum trend ratio, mean trend ratio, minimum sample size ratio, confidence interval and conditional power are calculated continuously at all points along the information time. In one embodiment, the invention discloses a graphical user interface-based method and system to early conclude a decision, i.e., futile, promising, sample size re-estimate, for an on-going clinical trial. In one embodiment, exact type I error rate control, median unbiased estimate of treatment effect, and exact two-sided confidence interval can be continuously calculated.

### CROSS-REFERENCE TO RELATED APPLICATIONS

This application is a continuation-in-part application of International Application No. PCT/IB2019/056613, filed Aug. 2, 2019, which claims the benefits of U.S. Ser. No. 62/807,584, filed Feb. 19, 2019 and U.S. Ser. No. 62/713,565, filed Aug. 2, 2018. The entire contents and disclosures of these prior applications are incorporated herein by reference into this application.

Throughout this application, various references are referred to and disclosures of these publications in their entireties are hereby incorporated by reference into this application to more fully describe the state of the art to which this invention pertains.

### FIELD OF THE INVENTION

Embodiments of the invention are directed towards systems, methods and processes for dynamic data monitoring and optimization of ongoing clinical research trials.

Using an electronic patient data management system such as commonly used EDC systems, treatment assignment system such as IWRS system and a specially designed statistical package, embodiments of the invention are directed towards a “closed system” or a graphical user interface (GUI) for dynamically monitoring and optimizing on-going clinical research trials or studies. This systems, methods and processes of the invention integrate one or more subsystems in a closed system thereby allowing the computation of the treatment efficacy score of the drug, medical device or other treatment in a clinical research trial without unblinding the individual treatment assignment to any subject or personnel participating in the research study. At any time during or after various phases of the clinical research study, as new data is cumulated, embodiments of the invention automatically estimate treatment effect, its confidence interval (CI), conditional power, updated stopping boundaries, and re-estimate the sample size as needed to achieve desired statistical power, and perform simulations to predict the trend of the clinical trial. The system can be also used for treatment selection, population selection, prognosis factor identification, signal detection for drug safety and connection with Real World Data (RWD) for Real World Evidence (RWE) in patient treatments and healthcare following approval of a drug, device or treatment.

### BACKGROUND OF THE INVENTION

In the United States, the Food and Drug Administration (the “FDA”) oversees the protection of consumers exposed to health-related products ranging from food, cosmetics, drugs, gene therapies, and medical devices. Under the FDA guidance, clinical trials are performed to test the safety and efficacy of new drugs, medical devices or other treatments to ultimately ascertain whether a new medical therapy is appropriate for the intended patient population. As used herein, the terms “drug” and “medicine” are used interchangeably and are intended to include, but are not necessarily limited to, any drug, medicine, pharmaceutical agent (chemical, small molecule, complex delivery, biologic, etc.), treatment, medical device or otherwise requiring the use of clinical research studies, trials or research to procure FDA approval. As used herein, the terms “study” and “trial” are used interchangeably and intended to mean a randomized clinical research investigation, as described herein, directed towards the safety and efficacy of a new drug. As used herein, the terms “study” and “trial” are further intended comprise any phase, stage or portion thereof.

Acronyms and Terms

| # | Acronym | Full Name, and Calculation |
|---|---|---|
| 1. | CI | Confidence Interval |
| 2. | DAD | Dynamic Adaptive Design |
| 3. | DDM | Dynamic Data Monitoring |
| 4. | IRT | Interactive Responding Technology |
| 5. | IWRS | Interactive Web-Responding System |
| 6. | RWE | Real-World Evidence |
| 7. | PV | Pharmacovigilance |
| 8. | TLFs | Tables, listing and figures |
| 9. | RWD | Real World Data |
| 10. | RCT | Randomized Clinical Trial |
| 11. | GS | Group Sequential |
| 12. | GSD | Group Sequential Design |
| 13. | AGSD | Adaptive GSD |
| 14. | DMC | Data Monitoring Committee |
| 15. | ISG | Independent statistical group |
| 16. | tₙ | Interim points |
| 17. | AGS | Adaptive Group Sequential |
| 18. | S, F | Stopping boundaries S (success) and F (failure) |
| 19. | SS | Sample size |
| 20. | SSR | Sample size re-estimation |
| 21. | z-score(s) | High efficacy score(s) |
| 22. | EDC | Electronic Data Capture |
| 23. | DDM | Dynamic Data Monitoring Engine |
| 24. | EMR | Electronic Medical Records |
| 25. | θ | Treatment effect size |
| 26. | N₀ | A planned/initial sample size (or “information” in general) N₀ (per arm) |
| 27. | α | Type-I error rate |
| 28. | H₀: θ = 0 | Null hypothesis |
| 29. | n_{E} and n_{C} | The number of subjects in the experimental group and in the control arm |
| 30. | X̅_{E,n}_{_{E}} | $\begin{matrix}Sample means in the experimental group , which is calculated by \\ \frac{1}{{n}_{E}} {\sum }_{i = 1}^{{n}_{E}} {X}_{E , i} \sim N ( {μ}_{E} , \frac{{σ}_{E}^{2}}{{n}_{E}} )\end{matrix}$ |
| 31. | X̅_{C,n}_{_{C}} | $\begin{matrix}Sample means in the control group , which is calculated by \\ \frac{1}{{n}_{C}} {\sum }_{i = 1}^{{n}_{C}} {X}_{C , i} \sim N ( {μ}_{C} , \frac{{σ}_{C}^{2}}{{n}_{C}} )\end{matrix}$ |
| 32. | Zₙ_{_{E}}_{,n}_{_{C}}, | $Wald statistics , which is calculated by ( {\overline{X}}_{E , {n}_{E}} - {\overline{X}}_{C , {n}_{C}} ) / \sqrt{\frac{{\hat{σ}}_{E}^{2}}{{n}_{E}} + \frac{{\hat{σ}}_{C}^{2}}{{n}_{C}}}$ |
| 33. | \hat{σ}_{E}²(\hat{σ}_{C}²) | Estimated variance for X_{E} (X_{C}) |
| 34. | iₙ_{_{E}}_{,n}_{_{C}} | $Estimated Fisher ’ s information , calculated by {( \frac{{\hat{σ}}_{E}^{2}}{{n}_{E}} + \frac{{\hat{σ}}_{C}^{2}}{{n}_{C}} )}^{- 1}$ |
| 35. | S(iₙ_{_{E}}_{,n}_{_{C}}) | $\begin{matrix}Score function , calculated by {Z}_{{n}_{E} , {n}_{C}} / \sqrt{\frac{{\hat{σ}}_{E}^{2}}{{n}_{E}} + \frac{{\hat{σ}}_{C}^{2}}{{n}_{C}}} = {Z}_{{n}_{E} , {n}_{C}} \sqrt{{i}_{{n}_{E} , {n}_{C}}} = \\ \hat{θ} {i}_{{n}_{E} , {n}_{C}} \cdot {S}_{{n}_{E} , {n}_{C}} \sim N ( θ {i}_{{n}_{E} , {n}_{C}} , {i}_{{n}_{E} , {n}_{C}} )\end{matrix}$ |
| 36. | CP(θ, N, C\|Sₙ_{_{E}}_{,n}_{_{C}}) | $\begin{matrix}Conditional Power \\ CP ( θ , N , C \vert u ) = P ( \frac{{S}_{N}}{\sqrt{{I}_{N}}} \geq C \vert {S}_{{n}_{E} , {n}_{C}} = u ) = 1 - Φ ( \frac{C \sqrt{{I}_{N}} - u - θ ( {I}_{N} - {i}_{{n}_{E} , {n}_{C}} )}{\sqrt{{I}_{N} - {i}_{{n}_{E} , {n}_{C}}}} ) ,\end{matrix}$ |
| 37. | \hat{θ} | $The point estimate , calculated by \frac{{S}_{{n}_{E} , {n}_{C}}}{{i}_{{n}_{E} , {n}_{C}}} \sim N ( θ , \frac{1}{{i}_{{n}_{E} , {n}_{C}}} ) or {\overline{X}}_{E , {n}_{E}} - {\overline{X}}_{C , {n}_{C}}$ |
| 38. | C | The critical/boundary value |
| 39. | C₁ | $\begin{matrix}Adjusted critical boundary value after sample size re \text{-} estimation , \\ calculated as {C}_{1} = \frac{1}{\sqrt{{I}_{{N}_{new}}}} \{ \frac{\sqrt{{I}_{{N}_{new}} - {i}_{{n}_{E} , {n}_{C}}}}{\sqrt{{I}_{{N}_{0}} - {i}_{{n}_{E} , {n}_{C}}}} ( {C}_{0} \sqrt{{I}_{{N}_{0}}} - u ) \} + \frac{u}{\sqrt{{I}_{{N}_{new}}}} , or as \\ {C}_{1} = \frac{1}{\sqrt{{T}_{1}}} \{ \frac{\sqrt{{T}_{1} - {t}_{0}}}{\sqrt{{T}_{0} - {t}_{0}}} ( {C}_{0} \sqrt{{T}_{0}} - {u}_{{t}_{0}} ) \} + \frac{{u}_{{t}_{0}}}{\sqrt{{T}_{1}}} .\end{matrix}$ |
| 40. | C_{g} | Final boundary value with O'Brien-Fleming boundary |
| 41. | r | $Information ratio ( \frac{{I}_{{N}_{new}}}{{I}_{{N}_{0}}} )$ |
| 42. | t | The information time (fraction) based on the originally planned |
| | | information I_{N}_{₀} at any iₙ_{_{E}}_{,n}_{_{C}}, i.e., iₙ_{_{E}}_{,n}_{_{C}}/I_{N}_{₀} |
| 43. | S(t) | The score function at information time t, where B(t)~N(0, t) is the |
| | | standard continuous Brownian motion process, calculated by |
| | | S(t) ≈ B(t) + θt~N(θt, t) |
| 44. | l | Total of the number of line segments examined |
| 45. | TR(l) | $\begin{matrix}Expected “ trend ratio ” of length l , calculated as \\ TR ( l ) = E ( \frac{1}{l} {\sum }_{i = 0}^{l - 1} sign ( S ( {t}_{i + 1} ) - S ( {t}_{i} ) ) )\end{matrix}$ |
| 46. | Mean TR | $\begin{matrix}Mean trend ratio , calculated as \\ \frac{1}{l - A + 1} ( {\sum }_{j = A}^{l} TR ( j ) ) = \frac{1}{l - A + 1} ( {\sum }_{j = A}^{l} \frac{1}{j} {\sum }_{i = 0}^{j - 1} sign ( S ( {t}_{i + 1} ) - S ( {t}_{i} ) ) ) , \\ wherein l represents the {l}^{th} block of patients to be monitored , A is the {1}^{st} \\ block to start of monitoring .\end{matrix}$ |
| 47. | mTR | $\begin{matrix}Maximum trend ratio ( mTR ) = {max}_{l} TR ( l ) , wherein TR ( l ) = \\ E ( \frac{1}{l} {\sum }_{i = 0}^{l - 1} sign ( S ( {t}_{i + 1} ) - S ( {t}_{i} ) ) ) , t = {i}_{{n}_{E} , {n}_{C}} / {I}_{{N}_{0}} as the information time \\ ( fraction ) based on the originally planned information {I}_{{N}_{0}} at any {i}_{{n}_{E} , {n}_{C}} ,\end{matrix}$ |
| 48. | τ | Time fraction when the SSR is conducted, τ = (number of patients |
| | | associated with the time of SSR)/total number of planed patients. |
| 49. | C₁ | $\begin{matrix}Adjusted critical boundary value after sample size re \text{-} estimation , \\ calculated as {C}_{1} = \frac{1}{\sqrt{{I}_{{N}_{new}}}} \{ \frac{\sqrt{{I}_{{N}_{new}} - {i}_{{n}_{E} , {n}_{C}}}}{\sqrt{{I}_{{N}_{0}} - {i}_{{n}_{E} , {n}_{C}}}} ( {C}_{0} \sqrt{{I}_{{N}_{0}}} - u ) \} + \frac{u}{\sqrt{{I}_{{N}_{new}}}} , or as \\ {C}_{1} = \frac{1}{\sqrt{{T}_{1}}} \{ \frac{\sqrt{{T}_{1} - {t}_{0}}}{\sqrt{{T}_{0} - {t}_{0}}} ( {C}_{0} \sqrt{{T}_{0}} - {u}_{{t}_{0}} ) \} + \frac{{u}_{{t}_{0}}}{\sqrt{{T}_{1}}} .\end{matrix}$ |
| 50. | C_{g} | Final boundary value with O'Brien-Fleming boundary |
| 51. | α(t) | $\begin{matrix}Continuous alpha \text{-} spending function , calculated by 2 \{ 1 - Φ ( {z}_{1 - α / 2} / \sqrt{t} ) \} , \\ 0 < t \leq 1 , to ensure the control of the type \text{-} I error rate\end{matrix}$ |
| 52. | bₖ | $\begin{matrix}Futility boundary value be {b}_{k} at information fraction time {t}_{k} = \frac{{i}_{k}}{{I}_{K}} , k = \\ 1 , … , K - 1. ( {i}_{K} = {I}_{k} and {t}_{K} = 1 ) . Thus the method would stop the \\ study at time {t}_{k} if {Z}_{k} \leq {b}_{k} and conclude futility for the test treatment\end{matrix}$ |
| 53. | ETI_{θ} | $\begin{matrix}Expected total information , calculated by \\ {\sum }_{k = 1}^{K - 1} {i}_{k} P ( stop at {t}_{k} for the first time \text{\textbar} θ ) + {I}_{K} P ( never stop at any interim \\ analysis \text{\textbar} θ ) = {I}_{K} {\sum }_{k = 1}^{K - 1} {t}_{k} P ( {Z}_{k} \leq {b}_{k} at {t}_{k} for the first time \text{\textbar} θ ) + \\ {I}_{K} P ( never stop at any interim analysis \text{\textbar} θ )\end{matrix}$ |
| 54. | CP_{TR(N)} | $\begin{matrix}Trend ratio based conditional power , calculated as \\ {CP}_{TR ( N )} = P ( \frac{S ( {I}_{N} )}{\sqrt{{I}_{N}}} \geq C \vert a \leq max \{ TR ( l ) , l = 10 , 11 , 12 , … \} < b ) , \\ where N = {N}_{0} or {N}_{new} is used .\end{matrix}$ |
| 55. | FR(t) | Futility ratio at time t, calculated by (number of points meeting |
| | | S(t) =<0)/(number of points of S(t) calculated) |
| 56. | f(θ) | $\begin{matrix}For inferences ( point estimate and confidence intervals ) . f ( θ ) is an \\ increasing function of θ , and f ( 0 ) is the p \text{-} value . It is defined as \\ f ( θ ) = {P ( \frac{S ( {T}_{0} )}{\sqrt{{T}_{0}}} \geq \frac{{u}_{{T}_{0}}}{\sqrt{{T}_{0}}} )}_{θ} = P ( B ( {T}_{0} ) + θ {T}_{0} \geq {u}_{{T}_{0}} ) = 1 - φ ( \frac{{u}_{{T}_{0}} - θ {T}_{0}}{\sqrt{{T}_{0}}} ) .\end{matrix}$ |
| 57. | u_{T}_{₀}^{BK} | $\begin{matrix}“ Backward image ” , calculated as \\ {u}_{{T}_{0}}^{BK} = \{ \frac{\sqrt{{T}_{1} - {t}_{0}}}{\sqrt{{T}_{0} - {t}_{0}}} ( {u}_{{T}_{1}} - {u}_{{t}_{0}} + θ ( {T}_{1} - {t}_{0} ) ) \} + {u}_{{t}_{0}} + θ ( {T}_{0} - {t}_{0} )\end{matrix}$ |
| 58. | PS(θ) | $\begin{matrix}Performance Score which is calculated by \\ PS ( θ ) = \{ \begin{matrix}- 1 , & ( {P}_{d} , {N}_{d} ) ∈ ( {A}_{1} ⋃ {A}_{2} ⋃ {A}_{3} ) \\ 0 , & ( {P}_{d} , {N}_{d} ) ∈ ( {B}_{1} ⋃ {B}_{2} ⋃ {B}_{3} ) \\ 1 , & ( {P}_{d} , {N}_{d} ) ∈ C\end{matrix}\end{matrix}$ |

On average, it takes at least ten years for a new drug to complete the journey from initial discovery to approval to the marketplace, with clinical trials alone taking six to seven years on average. The average cost to the research and development of each successful drug is estimated to be S2.6 billion. As discussed below, most clinical trials are comprised of three pre-approval phases: Phase I, Phase II and Phase III. Most clinical trials fail at Phase II and thus do not to advance to Phase III. Such failures occur for many reasons, but primarily include issues related to safety, efficacy and commercial viability. As reported in 2014, the success rate of any particular drug completing Phase II and advancing to Phase III is only 30.7%. See FIG. 1. The success rate of any particular drug completing Phase III and resulting in a New Drug Application (“NDA”) with the FDA is only 58.1%. In summary, only about 9.6% of drug candidates that were initially tested in human subjects (Phase I) were eventually approved by the FDA for use among the population. Importantly, in the pursuit of drug candidates that ultimately fail to obtain FDA approval, substantial sums of money are expended by the drug's sponsor. Even worse, in that process significant numbers of humans are unnecessarily and needlessly subjected to testing procedures for an ultimately futile drug candidate.

Once a new drug has undergone studies in animals and the results appear favorable, the drug can be studied in humans Before human testing may begin, findings of animal studies are reported to the FDA to obtain approval to do so. This report to the FDA is called an application for an Investigational New Drug (an “IND” and the application therefor, an “INDA” or “IND Application”).

The process of experimentation of the drug candidate on humans is referred to as a clinical trial, which generally involves four phases (three (3) pre-approval phases and one (1) post-approval phase). In Phase I, a few human research participants, referred to as subjects, (approximately 20 to 50) are used to determine the toxicity of the new drug. In Phase II, more human subjects, typically 50-100, are used to determine efficacy of the drug and further ascertain safety of the treatment. The sample size of Phase II trials varies, depending on the therapeutic area and the patent population. Some Phase II trials are larger and may comprise several hundred subjects. Doses of the drug are stratified to try to gain information about the optimal regimen. A treatment may be compared to either a placebo or another existing therapy. Phase III trials aim to confirm efficacy that has been suggested by results from Phase II trials. For this phase, more subjects, typically on the order of hundreds to thousands of subjects, are needed to perform a more conclusive statistical analysis. A treatment may be compared to either a placebo or another existing therapy. In Phase IV (post-approval study), the treatment has already been approved by the FDA, but more testing is performed to evaluate long-term effects and to evaluate other indications. That is, even after FDA approval, drugs remain under continued surveillance for serious adverse effects. The surveillance—broadly referred to as post-marketing surveillance—involves the collection of reports of adverse events via systematic reporting schemes and via sample surveys and observational studies.

Sample size tends to increase with the phase of the trial. Phase I and II trials are likely to have sample sizes in the 10s or low 100s compared to 100s or 1000s for Phase III and IV trials.

The focus of each phase shifts throughout the process. The primary objective of early phase testing is to determine whether the drug is safe enough to justify further testing in humans. The emphasis in early phase studies is on determining the toxicity profile of the drug and on finding a proper, therapeutically effective dose for use in subsequent testing. The first trials, as a rule, are uncontrolled (i.e., the studies do not involve a concurrently observed, randomized, control-treated group), of short duration (i.e., the period of treatment and follow-up is relatively short), and conducted to find a suitable dose for use in subsequent phases of testing. Trials in the later phases of testing generally involve traditional parallel treatment designs (i.e., the studies are controlled and generally involve a test group and a control group), randomization of patients to study treatments, a period of treatment typical for the condition being treated, and a period of follow-up extending over the period of treatment and beyond.

Most drug trials are done under an IND held by the “sponsor” of the drug. The sponsor is typically a drug company but can be a person or agency without “sponsorship” interests in the drug.

The study sponsor develops a study protocol. The study protocol is a document describing the reason for the experiment, the rationale for the number of subjects required, the methods used to study the subjects, and any other guidelines or rules for how the study is to be conducted. During clinical trials, participants are seen at medical clinics or other investigation sites and are generally seen by a doctor or other medical professional (also known as an “investigator” for the study). After participants sign an informed consent form and meet certain inclusion and exclusion criteria, they are enrolled in the study and are subsequently referred to as study subjects.

Subjects enrolled into a clinical study are assigned to a study arm in a random fashion, which is done to avoid biases that may occur in the selection of subjects for a trial. For example, if subjects who are less sick or who have a lower baseline risk profile are assigned to the new drug arm at a higher proportion than to the control (placebo) arm, a more favorable but biased outcome for the new drug arm may occur. Such a bias, even if unintentional, skews the data and outcome of the clinical trial to favor the drug under study. In instances where only one study group is present, randomization is not performed.

The Randomized Clinical Trial (RCT) design is commonly used for Phase II and III trials in which patients are randomly assigned the experimental drug or control (or placebo). The treatments are usually randomly assigned in a double-blind fashion through which doctors and patients are unaware which treatment was received. The purpose of randomization and double-blinding is to reduce bias in efficacy evaluation. The number of patients to be studied and the length of the trial are planned (or estimated) based on limited knowledge of the drug in early stage of development.

“Blinding” is a process by which the study arm assignment for subjects in a clinical trial is not revealed to the subject (single blind) or to both the subject and the investigator (double blind). Blinding, particularly double blinding, minimizes the risk of bias. In instances where only one study group is present, blinding is not performed.

Generally, at the end of the trial (or at specified interim time periods, discussed further below) in a standard clinical study, the database containing the completed trial data is transported to a statistician for analysis. If particular occurrences, whether adverse events or efficacy of the test drug, are seen with an incidence that is greater in one group over another such that it exceeds the likelihood of pure chance alone, then it can be stated that statistical significance has been reached. Using statistical calculations that are well known and utilized for such purposes, the comparative incidence of any given occurrence between groups can be described by a numeric value, referred to as a “p-value.” A p-value<0.05 indicates that there is a 95% likelihood that an incident occurred not due to the result of chance. In statistical context, the “p-value” is also referred to the false positive rate or false positive probability. Generally, FDA accepts the overall false positive rate <0.05. Therefore, if the overall p<0.05, the clinical trial is considered to be “statistically significant”.

In some clinical trials, multiple study arms, or even a control group, may not be utilized. In such cases, only a single study group exists with all subjects receiving the same treatment. This is typically performed when historical data about the medical treatment, or a competing treatment is already known from prior clinical trials and may be utilized for the purpose of making comparisons, or for other ethical reasons.

The creation of study arms, randomization, and blinding are well-established techniques relied upon within the industry and FDA approval process for determining safety and efficacy of a new drug. Such methods do present challenges, however, as these methods require the maintenance of the blinding to protect the integrity of a clinical trial, the clinical trial sponsor is prevented from tracking key information related to safety and efficacy while the study is ongoing.

One of the objectives of any clinical trial is to document the safety of a new drug. However, in clinical trials where randomization is conducted between two or more study arms, this can be determined only as a result of analyzing and comparing the safety parameters of one study group to another. When the study arm assignments are blinded, there is no way to separate subjects and their data into corresponding groups for purposes of performing comparisons while the trial is being conducted. Moreover, as discussed in greater detail, below, study data is only compiled and analyzed either at the end of the trial or at pre-determined interim analysis points, thereby subjecting study subjects to potential safety risks until such time that the study data is unblinded, analyzed and reviewed.

Regarding efficacy, any clinical trial seeking to document efficacy will incorporate key variables that are followed during the course of the trial to draw the conclusion. In addition, studies will define certain outcomes, or endpoints, at which point a study subject is considered to have completed the study protocol. As subjects reach their respective endpoints (i.e., as subjects complete their participation in the study), study data accrues along the study's information time line. These parameters, including both key variables and study endpoints, cannot be analyzed by comparison between study arms while the subjects are randomized and blinded. This poses potential challenges in ethics and statistical analysis.

Another related problem is statistical power. By definition, statistical power refers to the probability of a test appropriately rejecting the null hypothesis, or the chance of an experiment's outcome being the result of chance alone. Clinical research protocols are engineered to prove a certain hypothesis about a drug's safety and efficacy and disprove the null hypothesis. To do so, statistical power is required, which can be achieved by obtaining a large enough sample size of subjects in each study arm. When insufficient number of subjects are enrolled into the study arms, there exists the risk of the study not accruing enough subjects to reach statistical significance level to support the rejection of the null hypothesis. Because randomized clinical trials are usually blinded, the exact number of subjects distributed throughout study arms is not known until the end of the project. Although this maintains data collection integrity, there are inherent inefficiencies in the system, regardless of the outcome.

In a case where the study data reaches statistical significance for demonstrating efficacy or meeting futility criteria, as study subjects reach the endpoint of their participation in the study and study data accrues, an optimal time to close a clinical study would be at the very moment when statistical significance is achieved. While that moment may occur before the planned conclusion of a clinical trial, the time of its occurrence is generally not known. Thus, the trial would continue after its occurrence and the time and money spent beyond the occurrence would be unnecessary. Further, study subjects would continue to be enrolled above and beyond what is needed to reach the goals of the study, thereby placing human subjects under experimentation unnecessarily.

In a case where the study data it close to, but still falls short of, reaching statistical significance, generally there is a consensus that this is due to insufficient number of subjects being enrolled into the study. In such cases, to develop more supportive data, clinical trials will need to be extended. These extensions would not be possible if statistical analysis is performed only after a full closure of the study.

In a case where there is no trend toward significance, then there is little chance of reaching the desired conclusion even if more subjects are enrolled. In this case, it is desirable to close the study as early as possible once the conclusion can be established that the drug under investigation does not work and that continued study data has little chance of reaching statistical significance (i.e., continued investigation of the drug is futile). In randomized and blinded clinical trials, this trend would not be detected, and such conclusion of futility would not be made until final data analysis is conducted, typically at the end of trial or at pre-determined interim points. Again, in such cases, without the ability to detect the trend early, not only are time and money lost, but an excess of human subjects is placed under study unnecessarily.

To overcome such obstacles, clinical study protocols have implemented the use of interim analysis to help determine whether continued study is cost effective and ethical in terms of human testing. However, even such modified, sequential testing procedures may fall short of optimal testing since they necessarily require pre-determined interim timepoints, the experimentation periods between the interim analyses can be lengthy, study data needs to be unblinded, substantial time may be required for statistical analysis, etc.

FIG. 2 depicts a traditional “end of study analysis” randomized clinical trial design, commonly used for Phase II and III trials, where subjects are randomly assigned to either the drug (experimental) arm or the control (placebo) arm. In FIG. 2, the two hypothetical clinical trials are depicted for two different drugs (designated “Trial I” for the first drug and “Trial II” for the second drug). The center horizontal axis T designates the length of time (also referred to as “information time”) as each of the two trials proceed with trial information (efficacy results in terms of p-values) plotted for Trial I and Trial II. The vertical axis designates the efficacy score (commonly referred to as the “z-score”, e.g. the standardized difference of means) for the two trials. The start point for plotting study data along the information time T is at 0. Time continues along the information time axis T as the two studies proceed and study data (after statistical analysis) of both trials is plotted as it accrued with time. Both studies fully completed at line C (Conclusion—time of final analysis). The upper line S (“Success”) is the boundary for a statistically significant level of p<0.05. When (and if) accrued trial result data crosses S, a statistically significant level of p<0.05 is achieved, and the drug is deemed efficacious for the efficacy parameters defined in the study protocol. The lower line F (“Failure”) is the boundary for futility that indicates that the test drug is unlikely to have any meaningful efficacy. Both S and F are pre-calculated and established in the respective study's protocol. FIGS. 3-7 comprise similar efficacy score/information time graphs.

Continuing with FIG. 2, the hypothetical treatments of Trial I and Trial II were randomly assigned in a double-blinded fashion wherein neither the investigators nor the subjects knew whether the drug or the placebo was administered to subjects. The number of subjects that participated in each trial and the length of the trials were planned (or estimated) in the study protocol for the respective trial and were based on limited knowledge of the drugs in the earlier stages of their development. Upon completion C of the respective trials, the data accumulated during each trial is analyzed to determine whether the study objectives were met according to whether the results on primary endpoint(s) are statistically significant, i.e., p<0.05. At point C (the end of the trial), many trials—as those depicted in FIG. 2—are below the threshold of “success” (p<0.05) or are otherwise found to be futile. Ideally, such futile trials would have been terminated earlier to avoid unethical testing in patients and the expenditure of significant financial resources.

Continuing further with FIG. 2, the two trials depicted therein consist of a single time of data analysis, i.e., the conclusion of the trial at C. Trial I, while demonstrating a potentially successful drug candidate, still falls short of (below) S, i.e., the drug of Trial I has not met a statistically significant level of p<0.05 for efficacy. As for Trial I, a study involving more subjects or different dosage(s) could have resulted in p<0.05 for efficacy before the end of the trial; however, it was not possible for the sponsor to know of such fact until after Trial I concluded and the results analyzed. Trial II, on the other hand, should have been terminated earlier to avoid financial waste and unethically subjecting subjects to experimentation. This is demonstrated by the downward trend of the plotted efficacy score of the Trial II drug candidate away from a statistically significant level of p<0.05 for efficacy.

FIG. 3 depicts a randomized clinical trial design of two hypothetical Phase II or Phase III trials where subjects are randomly assigned to either the test drug (experimental) arm or the control (placebo) arm and wherein one or more interim data analyses are utilized. Specifically, the trials of FIG. 3 employ a commonly used Group Sequential (“GS”) design, wherein the study protocols incorporate one or more pre-determined interim statistical analyses of accumulated trial data while the trial is ongoing. This is unlike the design of FIG. 2, wherein study data is only unblinded, subjected to statistical analysis and reviewed after the study is complete.

Continuing with FIG. 3, points S and F are not single predetermined data points along line C. Rather, S and F are predetermined boundaries established in the study protocol and reflect the interim analysis aspect of the design. The upper boundary S, signifying that the drug's efficacy has achieved a statistically significant level of p<0.05 (and thus, the drug candidate is deemed efficacious for the efficacy parameters defined in the study protocol), and the lower boundary F, signifying that the drug is deemed a failure and further testing futile, are initially established as in FIG. 2. Unlike the data of the trials plotted in the graph of FIG. 2, however, wherein the results of neither trials are analyzed until the end of the trials at C, the stopping boundaries (both upper boundary S and lower boundary F) of the GS design of FIG. 3 are pre-calculated at predetermined interim points t₁ and t₂ (t₃, as depicted in FIG. 3, corresponds directly with study completion endpoint C). Upper boundary S and lower boundary F are precalculated at interim points t₁ and t₂ based on the rule that the overall false positive rate (α-level) must be <5%.

There are different types of flexible stopping boundaries. See, e.g., Flexible Stopping Boundaries When Changing Primary Endpoints after Unblinded Interim Analyses, Chen, Liddy M., et al, J BIOPHARM STAT. 2014; 24(4): 817-833; Early Stopping of Clinical Trials, at www.stat.ncsu.edu/people/tsiatis/courses/st520/notes/520chapter_9. pdf. One of the most commonly used flexible stopping boundaries is the O'Brien-Fleming boundary. As with the non-flexible boundaries of the non-interim trials of FIG. 2, with flexible boundaries, the upper boundary S, as pre-calculated, establishes efficacy (p<0.05) for the drug, whereas the lower boundary F, as pre-calculated, establishes failure (futility) for the drug.

Drug studies utilizing one or more interim analyses present certain obstacles. Specifically, clinical studies utilizing one or more interim data analyses must “unblind” study information in order to submit the data for appropriate statistical analyses. Drug trials without interim data analyses likewise unblind the study data—but at a point when the study has concluded, thereby mooting any potential for the intrusion of unwanted bias into the study's design and results. A drug trial using interim data analyses must, therefore, unblind and analyze the data in such a method and manner to protect the integrity of the study.

One means of properly performing the requisite statistical analyses of an interim based study is through an independent data monitoring committee (“DMC” or “IDMC”) that often works in conjunction with an independent third-party independent statistical group (“ISG”). At a predetermined interim data analyses, the accrued study data is unblinded through the DMC and provided to the ISG. The ISG then performs the necessary statistical analysis comparing the test and control arms. Upon competition of the statistical analysis of the study data, the results are returned to the DMC. The DMC reviews the results, and based on that review, the DMC makes various recommendations to the drug's sponsor concerning the continuation of the trial. Depending on the specific statistical analyses of a drug at an interim analysis (and the phase of study), the DMC may recommend continuing the trial, or that the experimentation be halted either due to likely futility; or, contrarily, the drug study has established the requisite statistical evidence of efficacy for the drug.

A DMC is typically comprised of a group of clinicians and biostatisticians appointed by a study's sponsor. According to the FDA's Guidance for Clinical Trial Sponsors—Establishment and Operation of Clinical Trial Data Monitoring Committees (DMC), “A clinical trial DMC is a group of individuals with pertinent expertise that reviews on a regular basis accumulating data from one or more ongoing clinical trials.” The FDA guidance further explains that “The DMC advises the sponsor regarding the continuing safety of trial subjects and those yet to be recruited to the trial, as well as the continuing validity and scientific merit of the trial.”

In the fortunate situation that the experimental arm is shown to be undeniably superior to the control arm, the DMC may recommend termination of the trial. This would allow the sponsor to seek FDA approval earlier and to allow the superior treatment to be available to the patient population earlier. In such case, however, the statistical evidence needs to be extraordinarily strong. However, there may be other reasons to continue the study, such as, for example, collecting more long-term safety data. The DMC considers all such factors when making its recommendation to the sponsor.

In the unfortunate situation that the study shows futility, the DMC may recommend that the trial be terminated. By way of example, if a trial is only one-half complete, but the experimental arm and the control arm have nearly identical results, the DMC may recommend that the study be halted. In this case, it is extremely unlikely that the trial, should it continue to its planned completion, would have the statistical evidence needed to obtain FDA approval of the drug. The sponsor would save money for other projects by abandoning the trial and other treatments could be made available for current and potential trial subjects. Moreover, future subjects would not undergo needless experimentation.

While a drug study utilizing interim data analysis has its benefits, there are downsides. First, there is the inherent risk that study data may be improperly leaked or compromised. While there have been no known incidences in which such confidential information was leaked or utilized by members of a DMC, cases have been suspected where such information was improperly used by individuals comprising or working for the ISG. Second, an interim analysis may require temporary stoppage of the study and use valuable time. Typically, an ISG may take between 3-6 months to perform its data analyses and prepare the interim results for the DMC. In addition, the interim data analysis is only a “snapshot” view of the study data at the interim analysis timepoint. While study data is statistically analyzed at various respective interim points (tₙ), trends in ongoing data accumulation are not typically investigated.

Referring again to FIG. 3, given the data results at interim information time points t₁ and t₂ of Trial I, the DMC would likely recommend to the sponsor of the drug of Trial I to continue further study. This conclusion is supported by the continued increase in the efficacy score of the drug, thereby continuing the study increases the likelihood of establishing an efficacy score that reaches statistical significance of p<0.05. The DMC may or may not recommend that Trial II continue. While the efficacy score of the drug of Trial II has decreased, Trial II has not crossed the line of failure—at least not yet. The data for Trial II is disappointing and may ultimately (and likely) be futile, but the DMC may nonetheless determine that the drug of Trial II warrants continued study. Unless the drug of Trial II had poor safety profile, it is possible that the DMC may recommend continued study.

In summary, although a GS design utilizes predetermined interim data analysis timepoints to statistically analyze and review the then-accrued study data at such timepoints, it nonetheless has various shortcomings. These include: 1) unblinding the study data in midstream to a third party, namely, the ISG, 2) the GS design only provides a “snapshot” of data at interim timepoints, 3) the GS design does not identify specific trends in accrual of trial data, 4) the GS design does not “learn” from the study data to make adaptations in study parameters to optimize the trial, 5) each interim analysis timepoint requires between 3-6 months for data analysis and preparation of interim data results.

The Adaptive Group Sequential (“AGS”) design is an improved version of the GS design, wherein interim data is analyzed and used to optimize (adjust) certain trial parameters or processes, such as sample size re-estimation and re-calculation of stopping boundaries, etc. By using this approach, it is possible to design a trial which can have any number of stages, begins with any number of experimental treatments, and permits any number of these to continue at any stage. In other words, an AGS design “learns” from interim study data, and as a result, adjusts (adapts) the original design to optimize the goals of the study. See, e.g., FDA Guidance for Industry (Draft Guidance), Adaptive Designs for Clinical Trials of Drugs and Biologics, September 2018, www.fda.gov/downloads/Drugs/Guidances/UCM201790.pdf. As with a GS design, an AGS design implements interim data analysis points, requires review and monitoring by a DMC, and requires 3-6 months for statistical analysis and result compilation.

FIG. 4 depicts an AGS trial design, again for the hypothetical drug studies, Trial I and Trial II. At predetermined interim timepoint t₁, study data for each trial is compiled and analyzed in the same fashion as that of the GS trial design of FIG. 3. Upon statistical analysis and review, however, various study parameters of each study may be adjusted, i.e., adapted for study optimization, thereby resulting in a recalculation of the upper boundary S and lower boundary F.

In the AGS study design of FIG. 4, data is compiled and analyzed for study adaptation, i.e., “learning & adaptation,” such as, for example, re-calculation of sample sizes, and thus, adjustment of stopping boundaries. As a result of such adaptations, e.g., modification of study sample sizes, boundaries are recalculated. At interim timepoint t₁ in FIG. 4, data is analyzed, and based on such analysis, study sample size is adjusted (increased). As a result of such modification, stopping boundaries S (success) and F (failure) are re-calculated. The initial boundaries of S₁ and F₂ are no longer used. Rather, commencing with interim timepoint t₁, stopping boundaries S₂ and F₂ are utilized. Continuing with FIG. 4, at predetermined interim timepoint t₂, study data is again compiled and analyzed. Once again, various study parameters may be adjusted (i.e., adapted for study optimization), e.g., modification of study sample size. In FIG. 4, study sample size is adjusted (increased) at interim timepoint t₂. As a result of such modification, stopping boundaries S₁ (success) and F₂ (failure) are re-calculated. Upper boundary S is recalculated an is now depicted as upper boundary S₃. Lower boundary F is recalculated and is now depicted as lower boundary F₃.

While the AGS design of FIG. 4 is an improvement over the GS design of FIG. 3, certain shortfalls remain. An AGS design still requires a DMC to review study data, thereby requiring a stoppage, albeit temporary, of the study at the predetermined interim time point and the unblinding of study data and the submission of that data to a third-party for statistical analysis, thereby presenting risk of comprising the integrity of study data. In addition, in an AGS design, data simulation is not performed to verify the validity and confidence of the interim results. As with a GS design, an AGS design still requires 3-6 months to complete the interim data analysis, review the results and make the appropriate recommendations. As with the GS design of FIG. 3, with the AGS design of FIG. 4, at the various interim timepoints the DMC could recommend that both Trial I and Trial II proceed, as both are within the various (and possibly adjusted) stopping boundaries. Or, the DMC could find, based on the specific data analyses presented to it, that Trial II be halted based on lack of efficacy. An obvious exception to proceeding with Trial II would also be if the drug of that study also exhibited a poor safety profile.

In summary, although an AGS design improves upon a GS design, it nonetheless has various shortcomings. These include: 1) unblinding the study data in midstream and providing same to a third party, namely, the ISG, 2) the AGS design still only provides a “snapshot” of data at interim timepoints, 3) the AGS design does not identify specific trends in accrual of trial data, 5) each interim analysis point requires between 3-6 months for data analysis and preparation of interim data results.

As noted above, the various interim timepoint designs of FIGS. 3 and 4 (GS and AGS) only present a “snapshot” of data at one or more pre-determined fixed interim timepoints to the DMC. Even after statistical analysis, such snapshot views could mislead the DMC and prevent optimal recommendations concerning the study at hand. What is desired, and what is provided in the embodiments of the invention, are methods, processes and systems of continuous data monitoring of trials whereby study data (efficacy and/or safety) is analyzed and recorded as it accrues in real time for subsequent review and consideration by the DMC at predetermined interim time points. As such, and after proper statistical analyses, the DMC would be presented with real-time results and study trends—as the data accrued—and thus be able to make better and optimal recommendations. A brief review of such continuous monitoring is instructive.

Referring to FIG. 5, a continuous monitoring design is depicted wherein study data for Trial I and Trial II are recorded or plotted along the T information time axis as such subject data accrues, i.e., as subjects complete the study. Each plot of study data undergoes full statistical analysis in relation to all data accrued at the time. Statistical analysis, therefore, does not wait for an interim timepoint tₙ, as in the GS and AGS designs of FIGS. 3-4 or the conclusion of trial design of FIG. 2. Rather, statistical analysis is ongoing in real time as study data accrues and the resultant data recorded in terms of efficacy score and/or safety along the information time axis T. At predetermined interim timepoints the entirety of the recorded data, in the graph format of FIGS. 5-7, is revealed to the DMC.

Referring specifically to FIG. 5, study data for Trial I and Trial II is compiled in real time, statistically analyzed and then recorded with subject endpoint accrual along information time axis T. At interim timepoint t₁, the recorded study data for both trials is revealed to and reviewed by the DMC. Based on the current status of study data, including trends in accrued study data, the DMC would be able to make more accurate and optimal recommendations as to both studies, including, but not limited to, adaptive recalculations of boundaries and/or other study parameters. As to Trial I in FIG. 5, the DMC would likely recommend continued study of the drug. As to Trial II, the DMC may find a trend towards low or lack of efficacy but would likely wait to the next interim timepoint for further consideration. In addition, the DMC may also find, based on reviewed study data, that sample size be adjusted, e.g., increased, and that stopping boundaries be re-calculated in accordance with the sample size modification.

Referring to FIG. 6, both Trial I and Trial II continue to interim timepoint t₂. Accrued study data is statistically analyzed in real time (as it accrues) in a closed environment and recorded in the same fashion as that described with respect to FIG. 5. At interim timepoint t₂, the continuously accrued, statistically analyzed and recorded study data of both Trial I and Trial II is revealed to and reviewed by the DMC. At interim timepoint t₂ in FIG. 6, the DMC would likely recommend that Trial I continue; sample size may or may not be adjusted (and thus, boundary S may or may not be re-calculated). At interim timepoint t₂ in FIG. 6, the DMC may find that it has convincing evidence, including the established trend of accrued study data, to recommend that the Trial II be terminated. This would be particularly so if the drug of Trial II has a poor safety profile. Possibly, depending on the specific statistical analysis available to the DMC with respect to Trial II, the DMC may recommend that the study continue, since the general trace of data in FIG. 6 shows the trial within the stopping boundaries.

Referring to FIG. 7, without continuous monitoring of Trial I and Trial II, the DMC could recommend that both studies continue, as both are within both stopping boundaries (S and F). Likely, the DMC would recommend that Trial II be terminated; again, however, any such recommendation would necessarily depend on the specific statistical analysis data reviewed by the DMC in accordance with a method, process and system that uses the real time statistical analysis of subject data as it accrues in a closed loop environment.

For ethical, scientific or economic reasons, most long-term clinical trials, especially those studying chronic diseases with serious endpoints, are monitored periodically so that the trial may be terminated or modified when there is convincing evidence either supporting or against the null hypothesis. The traditional group sequential design (GSD), which conducting tests at fixed time-points and pre-determined number of tests (Pocock, 1997; O'Brien and Fleming, 1979; Tsiatis, 1982) were much enhanced by the alpha-spending function approach (Lan and DeMets, 1983; Lan and Wittes, 1988; Lan and DeMets, 1989) with flexible test schedule and number of interim analyses during trial monitoring. Lan, Rosenberger and Lachin (1993) further proposed “occasional or continuous monitoring of data in clinical trials”, which, based on the continuous Brownian motion process, can improve the flexibility of GSD. However, due to logistic reasons, only occasional interim monitoring was performed in practice in the past. Data collection, retrieving, management and presentation to the Data Monitoring Committee (DMC), who conducts the interim looks, are all factors that hinder continuous data monitoring from practice.

The above GSD or continuous monitoring methods were very useful for making early study termination decision by properly controlling the overall type-I error rate, when the null hypothesis is true. The maximum information is pre-fixed in the protocol.

Another major consideration in clinical trial design is to estimate adequate amount of information needed to provide the desired study power, when the null hypothesis is not true. For this task, both the GSD and the fixed sample design depend on data from earlier trials to estimate the amount of (maximum) information needed. The challenge is that such estimate from external source may not be reliable due to perhaps different patient populations, medical procedures, or other trial conditions. Thus the prefixed maximum information in general, or sample size in specific, may not provide the desired power. In contrast, the sample size re-estimation (SSR) procedure, developed in the early 90's by utilizing the interim data of the current trial itself, aims to secure the study power via possibly increasing the maximum information originally specified in the protocol (Wittes and Britan, 1990; Shih, 1992; Gould and Shih, 1992; Herson and Wittes, 1993); see commentary on GSD and SSR by Shih (2001).

The two methods, GSD and SSR, later joined together and formed the so-called adaptive GSD (AGSD) by many authors during the last two decades, including Bauer and Kohne (1994), Proschan and Hunsberger (1995), Cui, Hung and Wang (1999), Li et al. (2002), Chen, DeMets and Lan (2004), Posch et al. (2005), Gao, Ware and Mehta (2008), Mehta et al. (2009), Mehta and Gao (2011), Gao, Liu and Mehta (2013), Gao, Liu and Mehta (2014), to just name a few. See Shih, Li and Wang (2016) for a recent review and commentary. AGSD has amended GSD with the capability of extending the maximum information pre-specified in the protocol using SSR, as well as possibly early termination of the trial.

### SUMMARY OF THE INVENTION

With SSR, there is still a critical issue of when the current trial data becomes reliable enough to perform a meaningful re-estimation. In the past, roughly the mid-trial time was suggested by practitioners as a principle, since there is no efficient continuous data monitoring tool available to analyze the data trend. However, mid-trial time-point is a snap shot which does not really guarantee data adequacy for SSR. Such a shortcoming can be overcome with data-dependent timing of SSR, based on continuous monitoring.

As the computing technology and computing power have drastically improved today, the fast transfer of data in real time is no longer an issue. Using the accumulating data for conducting continuous monitoring and timing the readiness of SSR by data trend will realize the full potential of AGSD. In this invention, this new procedure is termed as Dynamic Adaptive Design (DAD).

In this invention, the elegant continuous data monitoring procedure developed in Lan, Rosenberger and Lachin (1993) was expanded based on the continuous Brownian Motion process to DAD with a data-guided analysis for timing the SSR. DAD may be written in a study protocol as a flexible design method. When DAD is implemented as the trial is ongoing, it serves as a useful monitoring and navigation tool; this process is named as Dynamic Data Monitoring (DDM). In one embodiment, the terms of DAD and DDM may be used together or exchangeable in this invention, discloses a method of timing the SSR. In one embodiment, the overall type-I error rate is always protected, since both continuous monitoring and AGS have already been shown protecting the overall type-I error rate. It is also demonstrated by simulations that trial efficiency is much achieved by DAD/DDM in terms of making right decisions on either futility or early efficacy termination, or deeming trial as promising for continuation with sample size increase. In one embodiment, the present invention provides median unbiased point estimate and exact two-sided confidence interval for the treatment effect.

As for the statistical issues, the present invention provides a solution regarding how to examine a data trend and to decide whether it is time to do a formal interim analysis, how the type-I error rate is protected, the potential gain of efficiency, and how to construct a confidence interval on the treatment effect after the trial ends.

A closed system, method and process of dynamically monitoring data in an on-going randomized clinical research trial for a new drug is disclosed such that, without using humans to unblind the study data, a continuous and complete trace of statistical parameters such as, but not limited to, the treatment effect, the safety profiles, the confidence interval and the conditional power, may be calculated automatically and made available for review at all points along the information time axis, i.e., as data for the trial populations accumulates.

### BRIEF DESCRIPTION OF THE FIGURES

FIG. 1 is a bar graph that depicts approximate probabilities of success of drug candidates in various phases or stages in the FDA approval process based on historical data.

FIG. 2 depicts a graphical representation of efficacy of two hypothetical clinical studies of two drug candidates as measured by efficacy score along information time.

FIG. 3 depicts a graphical representation of efficacy and interim points of two hypothetical clinical studies of two drug candidates implementing a Group Sequential (GS) design.

FIG. 4 depicts a graphical representation of efficacy and interim points of two hypothetical clinical studies of two drug candidates implementing an Adaptive Group Sequential (AGS) design.

FIG. 5 depicts a graphical representation of efficacy and interim points of two hypothetical clinical studies of two drug candidates implementing a Continuous Monitoring design at interim point t₁.

FIG. 6 depicts a graphical representation of efficacy and interim points of two hypothetical clinical studies of two drug candidates implementing a Continuous Monitoring design at t₂.

FIG. 7 depicts a graphical representation of efficacy and interim points of two hypothetical clinical studies of two drug candidates implementing a Continuous Monitoring design at t₃.

FIG. 8 is a graphical schematic of an embodiment of the invention.

FIG. 9 is a graphical schematic of an embodiment of the invention depicting a work flow of a dynamic data monitoring (DDM) portion/system therein.

FIG. 10 is a graphical schematic of an embodiment of the invention depicting an interactive web response system/portion (IWRS) and electronic data capture (EDC) system/portion therein.

FIG. 11 is a graphical schematic of an embodiment of the invention depicting a dynamic data monitoring (DDM) portion/system therein.

FIG. 12 is a graphical schematic of an embodiment of the invention further depicting a dynamic data monitoring (DDM) portion/system therein.

FIG. 13 is a graphical schematic of an embodiment of the invention further depicting a dynamic data monitoring (DDM) portion/system therein.

FIG. 14 depicts graphical representations of statistical results of a hypothetical clinical study displayed as output by embodiments of the invention.

FIG. 15 depicts a graphical representation of efficacy of a promising hypothetical clinical study of a drug candidate displayed as output by embodiments of the invention.

FIG. 16 depicts a graphical representation of efficacy of a promising hypothetical clinical study of a drug candidate displayed as output by embodiments of the invention wherein subject enrollment is re-estimated and stopping boundaries are recalculated.

FIG. 17 is a schematic flow diagram showing representative steps of an exemplary implementation of an embodiment of the present invention.

FIG. 18 shows accumulative data from a simulated clinical trial according to one embodiment of the present invention.

FIG. 19 shows a trend ratio (TR) calculation according to one embodiment of the present invention

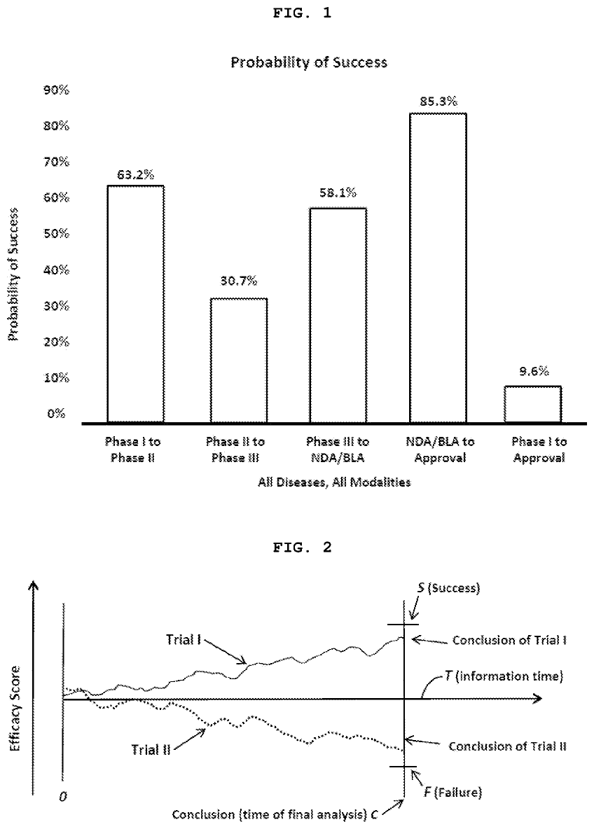

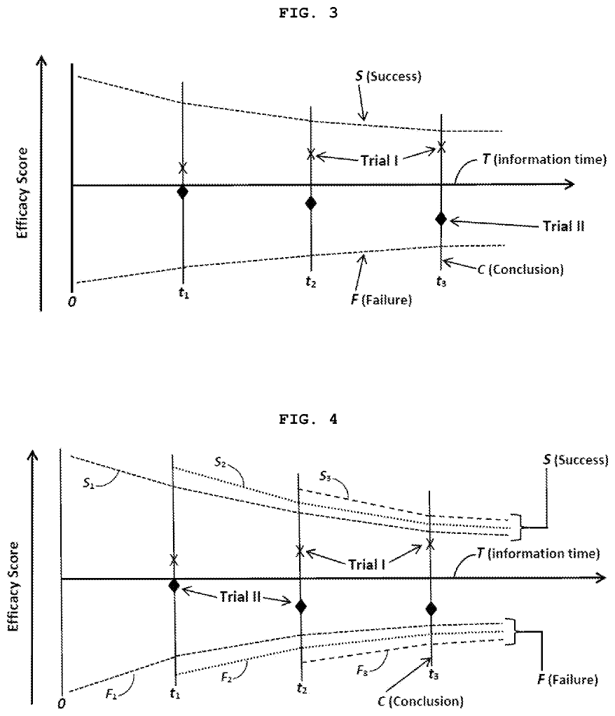

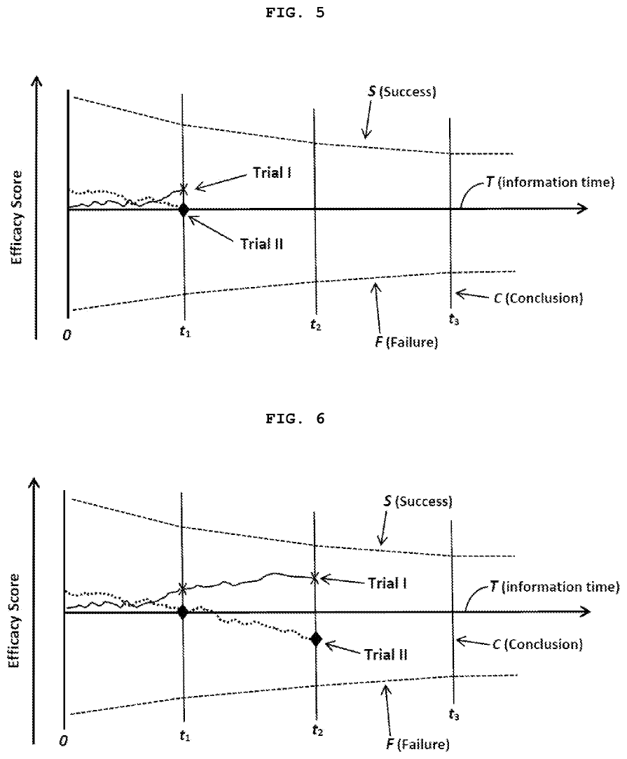

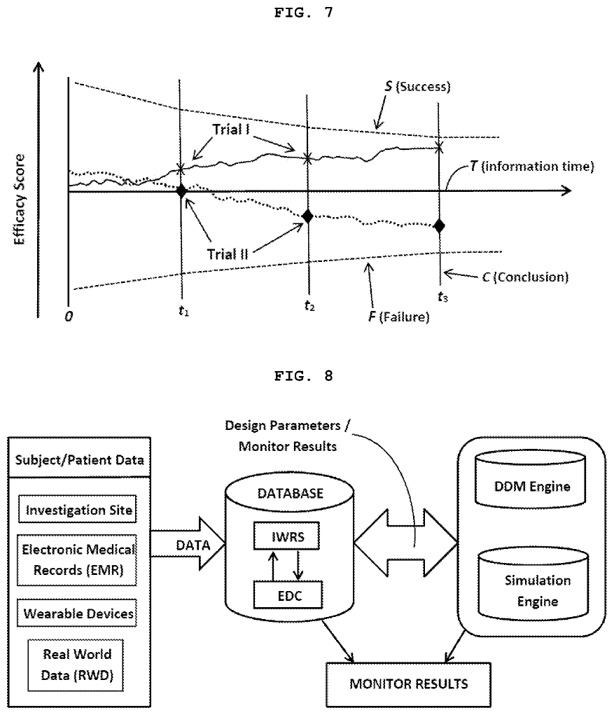

$$( TR ( l ) = \frac{1}{ι} {\sum }_{i = 0}^{ι - 1} sign ( S ( {t}_{i + 1} ) - S ( {t}_{i} ) ) ,$$

the calculation starts when l≥10, each time interval has 4 patients). The sign(S(tᵢ₊₁) −S(tᵢ)) is shown on the top row.

$$T R ( 10 ) = \frac{1}{10} {\sum }_{i = 0}^{9} sign ( S ( {t}_{i + 1} ) - S ( {t}_{i} ) ) = \frac{4}{10} = 0.4$$

$$T R ( 11 ) = \frac{1}{11} {\sum }_{i = 0}^{10} sign ( S ( {t}_{i + 1} ) - S ( {t}_{i} ) ) = \frac{5}{11} = 0.45$$

$$T R ( 12 ) = \frac{1}{12} {\sum }_{i = 0}^{11} sign ( S ( {t}_{i + 1} ) - S ( {t}_{i} ) ) = \frac{6}{12} = 0.5$$

$$T R ( 13 ) = \frac{1}{13} {\sum }_{i = 0}^{12} sign ( S ( {t}_{i + 1} ) - S ( {t}_{i} ) ) = \frac{5}{13} = 0.38$$

$$T R ( 14 ) = \frac{1}{14} {\sum }_{i = 0}^{13} sign ( S ( {t}_{i + 1} ) - S ( {t}_{i} ) ) = \frac{6}{14} = 0.43$$

$$T R ( 15 ) = \frac{1}{15} {\sum }_{i = 0}^{14} sign ( S ( {t}_{i + 1} ) - S ( {t}_{i} ) ) = \frac{5}{15} = 0.33$$

FIGS. 20A and 20B show a distribution of the maximum trend ratio, and a (conditional) rejection rate of Ho at the end of trial using maximum trend ratio CP_{mTR}, respectively.

FIG. 21 shows a graphical display of different performance score regions (sample size is Nₚ; Np0 is the required sample size for a clinical trial with a fixed sample size design, P₀ is the desired power. Performance score (PS)=1 is the best score, PS=0 is acceptable score, whereas PS=−1 is undesired score).

FIG. 22 shows the entire trace of Wald statistics for an actual clinical trial that eventually failed.

FIGS. 23A-23C show the entire trace of Wald statistics, Conditional power, and Sample Size Ratio, respectively, for an actual clinical trial that eventually succeeded.

FIG. 24 shows a representative GUI for dynamic monitoring of a clinical trial.

### DETAILED DESCRIPTION OF THE INVENTION

A clinical trial typically begins with a sponsor of the drug to undergo clinical research testing providing a detailed study protocol that may include items such as, but not limited to, dosage levels, endpoints to be measured (i.e., what constitutes a success or failure of a treatment), what level of statistical significance will be used to determine the success or not of the trial, how long the trial will last, what statistical stopping boundaries will be used, how many subjects will be required for the study, how many subjects will be assigned to the test arm of the study (i.e., to receive the drug), and how many subjects will be assigned to the control arm of the study (i.e., to receive either alternate treatment or placebo), etc. Many of these parameters are interconnected. For instance, the number of subjects required for the test group, and thus, receiving the drug, to provide the level of statistically significance required depends strongly on the efficacy of the drug treatment. If the drug is very efficacious, i.e., it is believed that the drug will achieve high efficacy scores (z-scores) and is predicted to achieve a level of statistical significance, i.e., p<0.05 early in the study, then significantly fewer patients will be required than if the treatment is beneficial, but at a lower degree of effectiveness. As the true effectiveness of the treatment is generally unknown for the study being designed, an educated guess about the effectiveness must be made, typically based on previous early phase studies, research publications or laboratory data of the treatments effect on biological cultures and animal models. Such estimates are built into the protocol of the study.

In embodiments, the study, and the design thereof based on the postulated effectiveness of the treatment, may proceed by randomly assigning subjects to either an experimental treatment (drug) or control (placebo or an active control or alternative treatment) arm. This may, for instance, be achieved using an Interactive Web Response System (“IWRS”) that may be a hardware and software package with build-in random number generator or pre-uploaded a list of random sequences. Enrolled subjects may be randomly assigned to either the treatment or control arm by the IWRS. The IWRS may contain subject's ID, treatment group assigned, date of randomization and stratification factors such as gender, age groups, disease stages, etc. This information will be stored in a database. This database may be secured by, for instance, suitable password and firewall protections such that the subject and the study investigators administering the study are unaware to which arm the subject has been assigned. Since neither subject nor investigator knows to which arm the subject has been assigned (and whether the subject is receiving the drug or a placebo or alternative treatment), the study, and the data resulting therefrom are effectively blinded. (To ensure blinding, for instance, both drug and placebo may be delivered in identical packaging but with encrypted bar codes, wherein only the IWRS database is able to direct the clinicians as to which package to administer to a subject. This may, therefore, be done without either the subject or the clinician being able to determine if it is the treatment drug or a placebo or an alternative treatment).

As the study progresses, subjects may be periodically evaluated to determine how the administered treatment is affecting them. This evaluation may be conducted by clinicians or investigators, either in person, or via suitable monitoring devices such as, but not limited to, wearable monitors, or home-based monitoring systems. Investigators and clinicians obtaining subjects' evaluation data may also be unaware to which study arm the subject was assigned, i.e., evaluation data is also blinded. This blinded evaluation data may be gathered using suitably configured hardware and software such as a server with Window or Linux operating system that may take the form of an Electronic Data Capture (“EDC”) system and may be stored in a secure database. The EDC data or database may likewise be protected by, for instance, suitable passwords and/or firewalls such that the data remains blinded and unavailable to participants in the study including subjects, investigators, clinicians and the sponsor.

In an embodiment of the invention, the IWRS for treatment assignment, the EDC for the evaluation database and Dynamic Data Monitoring Engine (“DDM”, a statistical analysis engine) may be securely linked to each other. This may, for instance, be accomplished by having the databases and the DDM all located on a single server that is itself protected and isolated from outside access, thereby forming a closed loop system. Or the secured databases and the secure DDM may communicate with each other by secure, encrypted communication links over a data communication network. The DDM may be equipped and suitably programmed such that it may obtain evaluation records from the EDC, and treatment assignment from the IWRS to calculate treatment effect, the score statistics, Wald statistics and 95% confidence intervals, conditional power and perform various statistical analysis without human involvement as such to maintain blindness of the trials to subjects, investigators, clinicians, the study sponsor or any other person(s) or entities.

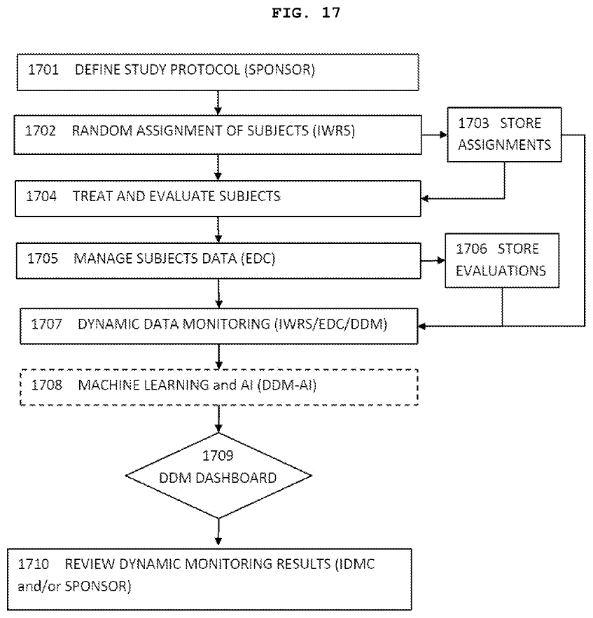

As the clinical trial proceeds in information time, i.e., as additional subjects in the study reach a trial endpoint and study data accrues, the closed system comprising the three interconnected software modules (EDC, IWRS and DDM) may perform continuous and dynamic data monitoring of internally unblinded data (discussed in greater detail, below, with respect to FIG. 17). The monitoring may include, but not limited to, computing the point estimate of efficacy score (i.e., the trace of cumulative treatment effect) and its 95% confidence interval, conditional power over the information time. Based on the data collected to date, the DDM may perform tasks including, but not limited to, calculating new sample size (number of subjects) needed to achieve desired statistical power, performing the trend analysis to predict the future of the study, performing analyses of study modification strategies, identifying optimal dose group so that the sponsor may consider to continue the study on the optimal dose group, identifying the subpopulation which is most likely to respond to the on the drug (treatment) under study so that further patient enrollment may only include such a subpopulation (population enrichment) and performing simulations on various study modification scenarios to estimate the success probability, etc.

Ideally, statistical analysis results, statistical simulations, etc. generated by the DDM on study data would be made available to the study's DMC and/or sponsor in real, or near real time, so that recommendations by the DMC can be made as early as practical and/or adjustments, modifications and adaptions can be made to optimize the study. For instance, a primary objective of a trial may be directed towards assessing the efficacy of three different dose levels of a drug against a placebo. Based on analysis by the DDM, it may become evident early in the trial that one of the dose levels is significantly more efficacious than either of the other two. As soon as that determination may be made by the DDM at a statistically significant level and made available to the DMC, it is advantageous to proceed further only with the most efficacious dose. This considerably reduces the cost of the study as now only one half of the subjects will be required for further study. Moreover, it may be more ethical to continue the treatment of all drug receiving subjects with the more efficacious dose rather than subjecting some of them to what is now reasonably known to be a less effective dose.

Current regulation allows such derived evaluations to be made available to the DMC prior to the study reaching a predetermined interim analysis time point, as discussed above, when all of the then-available study data may be unblinded to the ISG to perform interim analyses and present the unblinded results to the DMC. Upon receipt of analysis results, the DMC may advise the study's sponsor as to whether to continue and/or how to further proceed, and, in certain circumstances, may also provide guidance of recalculation of trial parameters such as, but not limited to, re-estimation of sample size and re-calculation of stopping boundaries.

The shortfalls of current practice include but are not limited to: (1) unblinding necessarily requires human involvement (e.g., the IS G), (2) preparation for and conducting the interim data study analysis by the ISG usually takes about 3-6 months, (3) thereafter, the DMC requires approximately two months prior to its DMC review meeting (wherein the DMC reviews the interim study data statistically analyzed by the ISG) to review the statistically analyzed study data the DMC received from the ISG (as such, at its DMC review meeting, the snapshot interim study data is about 5-8 months old).

The present invention can well address all these difficulties as above. The advantages of the present invention include, but not limited to, (1) the present closed system does not need human involvement (e.g., ISG) to unblind trial data; (2) the pre-defined analyses allow DMC and/or sponsor to review analysis results continuously in real time; (3) unlike conventional DMC practice where DMC reviews only snapshot of on-going clinical data, the present invention allows DMC to review the trace of data over patient accrual so that a more complete profile of safety and efficacy can be monitored; (4) the present invention can automatically perform sample-size re-estimation, update new stopping boundaries, perform trend analysis and simulations that predict the trial's success or failure.

Therefore, the present invention succeeds in conferring the desirable and useful benefits and objectives.

In one embodiment, the present invention provides a closed system and method for dynamically monitoring randomized, blinded clinical trials without using humans (e.g., the DMC and/or the ISG) to unblind the treatment assignment and to analyze the on-going study data.

In one embodiment, the present invention provides a display of a complete trace of the score statistics, Wald statistics, point estimator and its 95% confidence interval and the conditional power through information time (i.e., from commencement of the study through most recent accrual of study data).

In one embodiment, the present invention allows the DMC, sponsor or any others to review key information (safety profiles and efficacy scores) of on-going clinical trials in real time without using ISG thus avoiding a lengthy preparation.

In one embodiment, the present invention is to use machine learning and AI technology in the sense of using the observed accumulated data to make intelligent decision, to optimize clinical studies so that their chance of success may be maximized.

In one embodiment, the present invention detects, at a stage as early as possible, “hopeless” or “futile” trials to prevent unethical patient suffering and/or multi-millions-dollar financial waste.

A continuous data monitoring procedure as described and disclosed by the present invention (such as DAD/DDM) for a clinical trial provides advantages in comparison to the GSD or AGSD. A metaphor is used here for easy illustration. A GPS navigation device is commonly used to guide drivers to their destinations. There are basically two kinds of GPS devices: build-in GPS for automobiles (auto GPS) and smart phone GPS. Typically, the auto GPS is not connected to the internet and does not incorporate traffic information, thus the driver can be stuck in heavy traffic. On the other hand, a phone GPS that is connected to the internet can select the route with the shortest arrival time based on the real time traffic information. An auto GPS can only conduct a fixed and inflexible pre-planned navigation without using the real time information. In contract, a phone GPS app uses up-to-the minute information for dynamic navigation.

The GSD or AGSD selects time points for interim analyses without knowing when or whether the treatment effect is stable as at the time of analysis. Therefore, the selection of time points for interim analyses could be pre-mature (thus giving an inaccurate trial adjustment) or late (thus missing the opportunity for a timely trial adjustment). In this invention, the DAD/DDM with real-time continuous monitoring after each patient entry is analogous to the smart phone GPS that can guide the trial's direction in a timely fashion with immediate data input from the trial as it proceeds.

As for the statistical issues, the present invention provides a solution on how to examine a data trend and to decide whether it is time to do a formal interim analysis, how the type-I error rate is protected, the potential gain of efficiency, and how to construct a confidence interval on the treatment effect after the trial ends.

Embodiments of the present invention will now be described in more detail with reference to the drawings in which identical elements in the various figures are, as far as possible, identified with the same reference numerals. These embodiments are provided by way of explanation of the present invention, which is not, however, intended to be limited thereto. Those of ordinary skill in the art may appreciate upon reading the present specification and viewing the present drawings that various modifications and variations may be made thereto without departing from the spirit of the invention.

The within description and illustrations of various embodiments of the invention are neither intended nor should be construed as being representative of the full extent and scope of the present invention. While particular embodiments of the invention are illustrated and described, singly and in combination, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited in any fashion. Rather, particular, detailed and exemplary embodiments are presented.

The images in the drawings are simplified for illustrative purposes and are not necessarily depicted to scale. To facilitate understanding, identical reference numerals are used, where possible, to designate substantially identical elements that are common to the figures, except that suffixes may be added, when appropriate, to differentiate such elements.

Although the invention herein has been described with reference to particular illustrative and exemplary physical embodiments thereof, as well as a methodology thereof, it is to be understood that the disclosed embodiments are merely illustrative of the principles and applications of the present invention. Therefore, numerous modifications may be made to the illustrative embodiments and other arrangements may be devised without departing from the spirit and scope of the present invention. It has been contemplated that features or steps of one embodiment may be incorporated in other embodiments of the invention without further recitation.

FIG. 17 is a schematic flow diagram showing representative steps of an exemplary implementation of an embodiment of the present invention.

In Step 1701, DEFINE STUDY PROTOCOL (SPONSOR), a sponsor such as, but not limited to, a pharmaceutical company, may design a clinical research study to determine if a new drug is effective for a medical condition. Such a study typically takes the form of a random clinical trial that is preferably double-blinded as previously described. Ideally the investigator, clinician, or care giver, administering the treatment shall also be unaware as to whether the subject is being administered the drug or a control (placebo or alternative treatment), although safety issues, or if the treatment is a surgical procedure, sometime make this level of blinding impossible or undesirable.

The study protocol may specify the study in detail, and in addition to defining the objectives, rationale and importance of the study, may include selection criteria for subject eligibility, required baseline data, how the treatment is to be administered, how the results are to be collected, and what constitutes an endpoint or outcome, i.e., a conclusion that an individual subject has completed the study, has been effectively treated or not, or such other defined endpoint. The study protocol may also include an estimation of the sample size that is necessary to achieve a meaningful conclusion. For both cost minimization and reduced exposure of subjects to experimentation, it may be desirable to implement the study utilizing the minimum number of subjects, i.e., using the smallest sample size while seeking to achieve statistically meaningful results. The trial design may, therefore, rely heavily on complex, but proven to be valid, statistical analysis of raw study data. For this and other reasons, clinical research studies or trials typically assess a single type of intervention in a limited and controlled setting to make analysis of raw study data meaningful.

Nevertheless, the sample size necessary to establish a statistically significant conclusion of efficacy such as “superiority” or “inferiority” over a placebo or standard or alternative treatment may depend on several parameters, which are typically specified and defined in the study protocol. For example, the estimated sample size required for a study is typically inversely proportional to the anticipated intervention effect or efficacy of the treatment of the drug. The intervention effect is, however, not generally well known at the start of the study—it is the variable being determined—and may only be approximated from laboratory data based on the effect on cultures, animals, etc. As the trial progresses, the intervention effect may become better defined, and making adjustments to the trial protocol may become desirable. Other statistical parameters that may be defined in the protocol include the conditional power; stopping boundaries that may be based on the P-value or level of significance—typically taken to be <0.05; the statistical power, population variance, dropout rate and adverse event occurrence rate.

In Step 1702, RANDOM ASSIGNMENT OF SUBJECTS (IWRS), eligible subjects may be randomly assigned to a treatment group (arm). This may, for instance, be done using the interactive web-based responding system, i.e., IWRS. The IWRS may use a pre-generated randomization sequence or a build-in random generator to randomly assign subjects to a treatment group. When a subject's treatment group is assigned, a drug label sequence corresponding to the treatment group will also assigned by the IWRS so that the correct study drug may be dispensed to the subject. The randomization process is usually operated by study site, e.g., a clinic or hospital. The IWRS may also, for instance, enable the subject to resister for the study from home via a mobile device, a clinic or a doctor's office.

In Step 1703, STORE ASSIGNMENTS, the IWRS may store the randomization data such as, but not limited to, subject ID (identification), treatment arm, i.e., test (drug) vs. control (placebo), stratification factors, and/or subject's demographic information in a secured database. This data linking subject identity to treatment group (test or control) may be blinded to the subject, investigators, clinicians, caregivers and sponsor involved in conducting the study.

In Step 1704, TREAT AND EVALUATE SUBJECTS, study drug, or placebo or an alternative treatment in accordance with the assignment may be dispensed to the subject right after the subject was randomized Subjects are required to follow study visit schedule to return to the study site for evaluation. The number and frequency of visits are well defined in study protocol. Type of evaluation, such as vital signs, lab tests, safety and efficacy assessments, will be performed according to study protocol.

In Step 1705, MANAGE SUBJECTS DATA (EDC), an investigator, clinician or caregiver may evaluate a trial subject in accordance with guidelines stipulated in the study protocol. The evaluation data may then be entered in an Electronic Data Capture (EDC) system. The collection of evaluation data may also/or instead include the use mobile devices such as, but not limited to, wearable physiological data monitors.

In Step 1706, STORE EVALUATIONS, the evaluation data collected by the EDC system may be stored in an evaluation database. An EDC system must comply with federal regulation, e.g., 21 CFR Part 11 to be used for managing clinical trial subjects and data.

In Step 1707, DYNAMIC DATA MONITORING, the DDM system or engine may be integrated with the IWRS and the EDC to form a closed system to analyze unblinded data. The DDM may access data in both the blinded assignment database and the blinded evaluation database DDM engine computes treatment effect and 95% confidence interval, conditional power, etc. over the information time and displays the results on a DDM dashboard. The DDM may also perform trend analysis and simulations using the unblinded data while the study is ongoing.

The DDM system may, for instance, include a suite of suitably programmed statistical modules such as a function in R-language to compute the conditional power that may allow the DDM to automatically make up-to-date, near real-time calculations such as, but not limited to, a current estimate of efficacy scores, and statistical data such as, but not limited to, a conditional power of the current estimate of efficacy and a current confidence interval of the estimate. The DDM may also make statistical simulations that may predict, or help predict, the future trend of the trial based on the accrued study data collected to date. For example, at a specific time of data accrual, the DDM system may use the observed data (enrollment rate and pattern, treatment effect, trend) to simulate outcome for future patients. The DDM may use those modules to produce a continuous and complete trace of statistical parameters such as, but not limited to, the treatment effect, the confidence interval and the conditional power. These and other parameters may be calculated and made available at all points along the information time axis, i.e., as endpoint data for the trial populations accumulates.

Step 1708, MACHINE LEARNING AND AI (DDM-AI), at this step, the DDM will use the machine learning and AI technology to optimize the trial in order to maximize the success rate as described above, particularly in the paragraph [0088].

In Step 1709, DDM DASHBOARD, DDM dashboard is a graphical user interface operable with EDC, which displays dynamic monitoring results (as described in this invention). DMC and/or sponsor or authorized personnel can have access to the dashboard.

In Step 1710, DMC may review the dynamic monitoring results any time. DMC can also request for a formal data review meeting if there is any safety concern signal or efficacy boundary crossing. DMC can also make a recommendation whether the clinical trial shall continue or stop. If there is a recommendation to make, DMC will discuss with sponsor. Under certain restriction and compliance of regulation, the sponsor may also review the dynamic monitoring results.

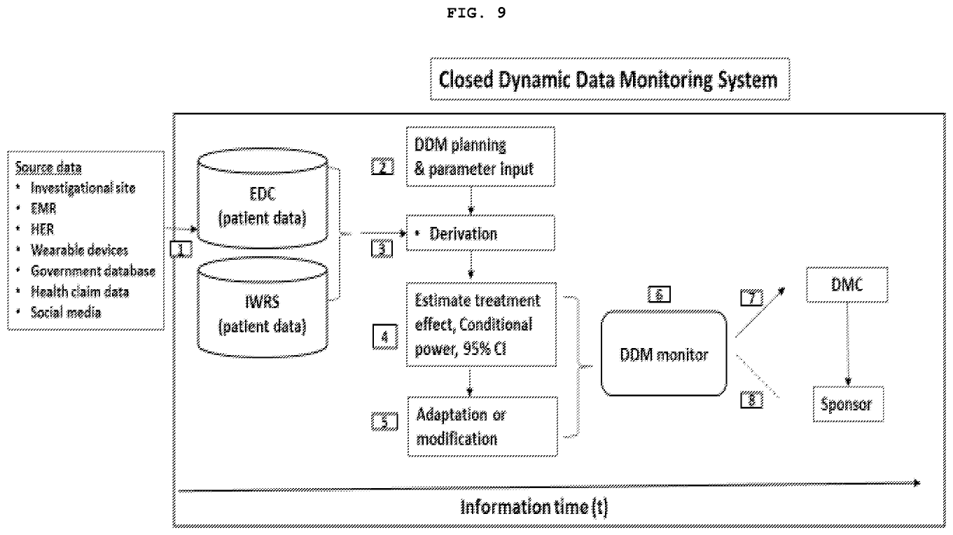

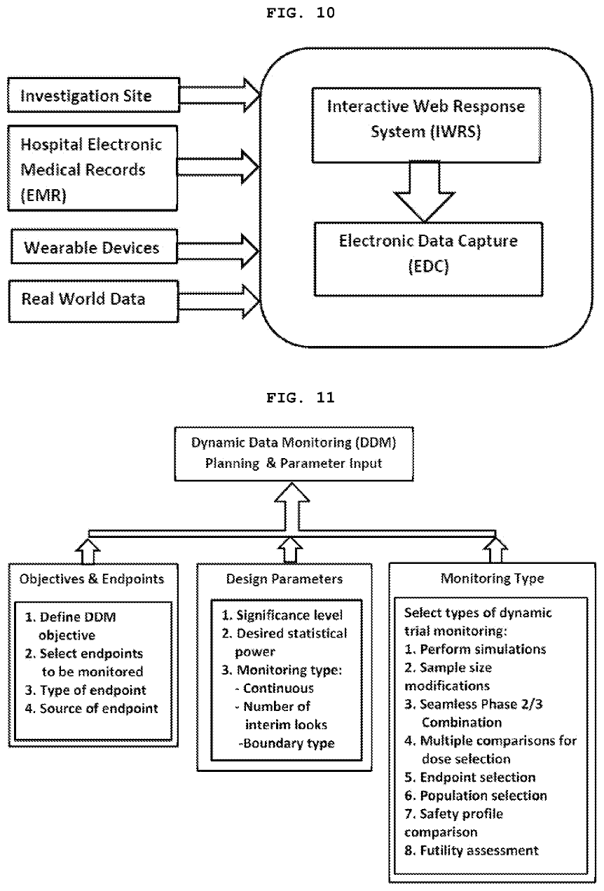

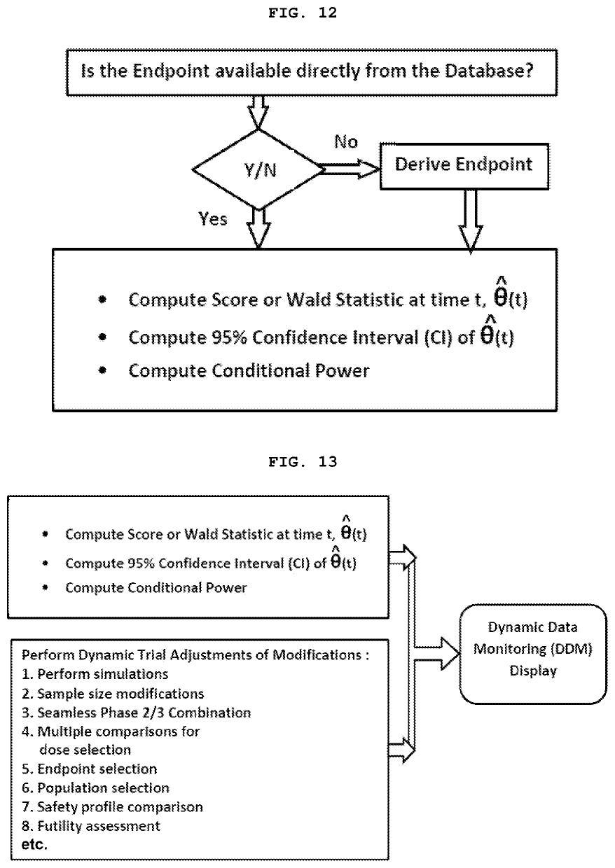

FIG. 8 shows a DDM system according to one embodiment of the present invention.

As shown, the system of the present invention may integrate multiple subsystems into a closed loop so that it may compute the score of treatment efficacy without human's involvement in unblinding individual treatment assignment. At any time as new trial data is accumulated, the system automatically and continuously estimates treatment effect, its confidence interval, conditional power, updated stopping boundaries, and re-estimate the sample size needed to achieve a desired statistical power, and perform simulations to predict the trend of the clinical trial. The system may be also used for treatment selection, population selection, prognosis factor identification and connection with Real World Data (RWD) for Real World Evidence (RWE) in patient treatments and healthcare. In one embodiment, the monitor results as shown in FIG. 8 are exported to a graphical user interface (GUI) and such GUI comprises a menu allowing a user to select one or more statistical quantities to be displayed. In one embodiment, GUI comprises a subsection showing whether the on-going clinical trial is promising or hopeless. In one embodiment, GUI comprises a subsection showing whether sample size adjustment is needed.

In some embodiments, the DDM system of the invention comprises a closed system consisting an EDC system, an IWRS and a DDM integrated into a single closed loop system. In one embodiment, such integration is essential to ensure that the use of treatment assignment for calculating treatment efficacy (such as the difference of means between treatment group and control group) may remain within the closed system. The scoring function for different types of endpoint may be built inside the EDC or inside DDM engine.

FIG. 9 shows a schematic representation of DDM system and the work flow (Component 1: Data Capture; Component 2: DDM Planning and Configuration; Component 3: Derivation; Component 4: Parameter Estimation; Component 5: Adaption and Modification; Component 6: Data Monitoring; Component 7: DMC Review; Component 8: Sponsor Notification. In one embodiment, the Data Monitoring (component 6) is a graphical user interface (GUI) as described in this invention.

In one embodiment, as shown in FIG. 9, the DDM system operates in the following manner:
At any time, t (t is referred to the information time during the trial), the efficacy score z(t) up to time t may be calculated within the EDC system or DDM engine;
The z(t) may be delivered to the DDM engine to compute the conditional power (probability of success) at t;
The DDM engine may also perform N (e.g., N>1000) times of simulations using the observed efficacy score z(t) to predict the trend of the clinical trial, for example, using observed z(t) and its trend for first 100 patients, simulate 1000 more patients with the same pattern to predict the future performance of the trial;
This process may be dynamically executed as the trial progresses;
The process may be used for many purposes such as population selection and prognosis factor identification.

FIG. 10 shows Component 1 of the system in FIG. 9 according to one embodiment of the present invention.

FIG. 10 illustrates how patient data may be entered into the EDC system. The source of the data may include but not limited to, an entity such as an investigator site, hospital Electronic Medical Records (EMR), wearable devices, that may transmit the data directly to the EDC, real world data, such as, but not limited to, governmental data, insurance claim data, social medias, or some combination thereof. This data may all be captured by the EDC system.

Subjects enrolled in the study may be randomly assigned to treatment groups. For double-blind, randomized clinical trials, the treatment assignment should not be disclosed to anyone involved in conducting the trial during the entire course of the trial. Typically, the IWRS keeps the treatment assignment separate and secure. In a conventional DMC monitoring practice, only a snapshot of study data at a predefined intermediate point may be disclosed to the DMC. The ISG then typically requires approximately 3-6 months to prepare the interim analysis results. This practice requires significant human involvement and may create potential risk of unintentional “unblinding”. These may be considered as major disadvantages in current DMC practice. The closed systems of embodiments of the present invention for performing interim data analyses of ongoing studies are thus preferable over current DMC practice.

FIG. 11 shows a schematic representation of a second portion (Component 2 in FIG. 9) according to one embodiment of the present invention).

As shown in FIG. 11, a user, e.g., a study's sponsor, may need to specify the endpoints that may be monitored. Endpoints are typically definable, measurable outcomes that may result from the treatment of the subject of the study. In one embodiment, multiple endpoints may be specified, such as one or more primary efficacy endpoints, one or more safety endpoints, or any combination thereof. In one embodiment, the endpoints subject to monitoring are selected by a user on a menu of a graphical user interface (GUI). In one embodiment, the user may select one or more statistical quantities for the monitoring.

In one embodiment, in selecting the endpoints to be monitored, the type of the endpoint can also be specified, i.e., if it may be analyzed using a particular type of statistic such as, but not limited to, as a normal distribution, as a binary event, as a time-to-event, or as a Poisson distribution, or any combination thereof.

In one embodiment, the source of the endpoint can also be specified, i.e., how the endpoint may be measured and by whom and how it may be determined that an endpoint has been reached.

In one embodiment, the statistical objectives of the DDM can also be defined. This may for instance, be accomplished by the user specifying one or more study, or trial, design parameters such as, but not limited to, a statistical significance level, a desired statistical power, and a monitoring type such as, but not limited to, continuous monitoring or frequent monitoring, including a frequency of such monitoring.

In one embodiment, one or more interim looks are specified, i.e., stopping points that may be based on information time or percent patient accrual, when the trial may be halted and data may be unblinded and analyzed. The user may also specify the type of stopping boundary to be used such as a boundary based on Pocock type analysis, one based on an O'Brien-Fleming type analysis, the user's choice or on alpha spending, or some combination thereof.

The user may also specify a type of dynamic monitoring, including actions to be taken such as, but not limited to, performing simulations, making sample size modifications, attempting to perform a seamless Phase 2/3 trial combination, making multiple comparisons for dose selection, making endpoint selection and adjustment, making trial population selection and adjustment, making a safety profile comparison, making a futility assessment, or some combination thereof.

FIG. 12 shows a schematic flow chart of actions that may be accomplished using Components 3 and 4 in FIG. 9 according to some embodiments of the invention.

In these components, the endpoint data of the treatment being investigated may be analyzed. If the endpoint to be monitored is not directly available from the database, the system may, for instance, require a user to enter one or more endpoint formulas such as blood pressures, laboratory tests that may be used to derive the endpoint data from the available data. These formulas may be programmed into the system within the closed loop of the system.

Once the endpoint data is derived, the system may automatically compute statistical information using the endpoint data, such as, but not limited to, a point estimate (t) at information time t, its 95% confidence level or confidence interval (CI), the conditional power as a function of patient accrual, or some combination thereof.

FIG. 13 shows a tabulation of representative pre-specified types of monitoring that may be performed in Component 6 of the system in FIG. 9.

As shown in FIG. 13, at this juncture one or more pre-specified types of monitoring may be performed by the DDM engine, and the results displayed on, for instance, a DDM display monitor or video screen. In one embodiment, the DDM display is a graphical user interface as described in this invention. The tasks may, for instance, be tasks, such as, but not limited to, performing simulations, making sample size modifications, attempting to produce a seamless Phase 2/3 combination, making multiple comparisons for dose selection, making an endpoint selection, making a population selection, making a safety profile comparison, making a futility assessment, or some combination thereof.

The results of the DDM engine may be output in graphic or tabular form, or some combination thereof, and may, for instance, be displayed on a monitor, or video screen.

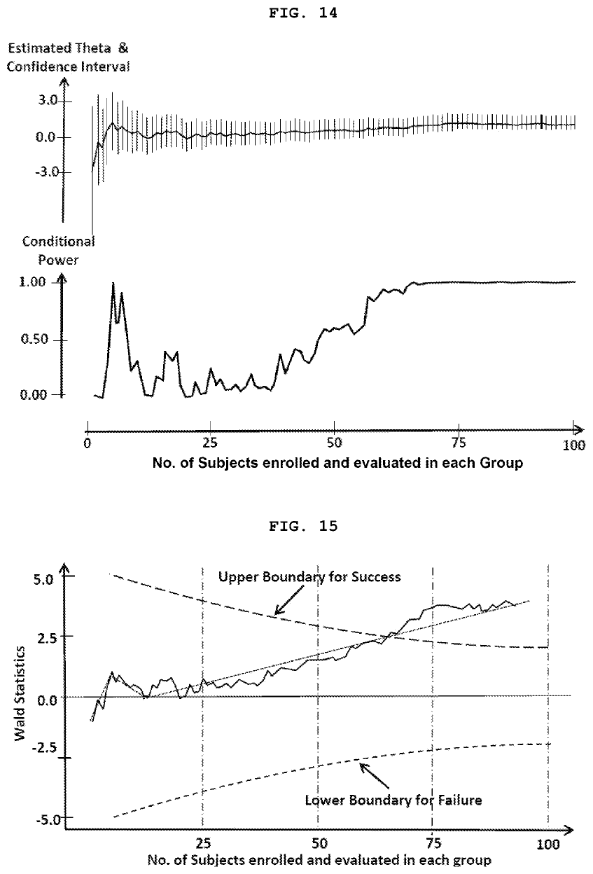

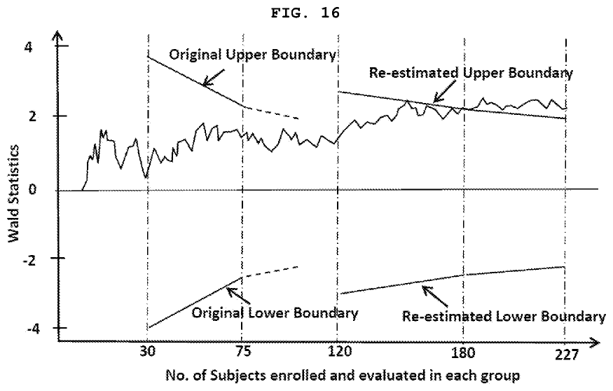

FIGS. 14 and 15 show exemplary graphical output from a DDM engine analysis of a promising trial. In one embodiment, such graphical output is subsection of the GUI. In one embodiment, such graphical output is displayed on a display parallel to the GUI.

Items displayed in FIGS. 14 and 15 include the estimated efficacy as a function of patient accrual, or information time, overlaid with the 95% confidence interval CI of the data points, and the Conditional Power as a function as well patient accrual, or information time, overlaid with O'Brien-Fleming analysis stopping boundaries. As seen from the plots of FIGS. 14 and 15, this simulated trial could have been stopped early at about the 75% patient accrual mark as by that point in the trial, the efficacy of the treatment had been proven to a statistically satisfactory degree.

FIG. 16 shows, in graphical form, representative results from a DDM engine analysis of a trial in which adaptations were made.

As shown in FIG. 16, the Adaptive Sequential Design began with an initial sample size of 100 patients per arm, or treatment group, and with pre-planned interim looks, or analysis, of unblinded data at the 30% and the 75% patient accrual points. As shown, a sample size re-estimation was performed at 75% patient accrual. The re-estimated sample was 227 per arm. Another two interim looks were planned at 120 and 180 patient accrual points. The trial crossed the updated stopping boundary for success when endpoint data on 180 patients had been accrued. If this trial had only been carried through to the initial goal of obtaining endpoint data on 100 patients, it would most likely fall slightly short of being a successful study as a statistically significant result may not have been arrived by that point. So, the trial could have failed had it been conducted based purely on the initial trial design. The trial, however, eventually became successful because of the continuous monitoring and the adaptation of a sample size estimation that the continuous monitoring enabled.

In one embodiment, the present invention provides a method of dynamically monitoring and evaluating an on-going clinical trial associated with a disease or condition, the method comprising:
1) collecting blinded data by a data collection system from the clinical trial in real time,
2) automatically unblinding the blinded data by an unblinding system operable with the data collection system into unblinded data,
3) continuously calculating statistical quantities, threshold values, and success and failure boundaries by an engine based on the unblinded data, and
4) outputting an evaluation result indicating one of the following:
   the clinical trial is promising, and
   the clinical trial is hopeless and should be terminated,
wherein the statistical quantities are selected from one or more from Score statistics, point estimate (\hat{θ}) and its 95% confidence interval, Wald statistics (Z(t)), conditional power (CP(θ,N,C|μ)), maximum trend ratio (mTR), sample size ratio (SSR), and mean trend ratio.

In one embodiment, the clinical trial is promising when one or more of the following are met:
(1) value of maximum Trend Ratio (mTR) is in a range of in (0.2, 0.4),
(2) value of mean trend ratio is no less than 0.2,
(3) value of the score statistics is constantly trending up or are constant positive along information time,
(4) the slope of a plot of Score Statistics vs information time is positive, and
(5) a new sample size is no more than 3 folds of the sample size as planned.

In one embodiment, the clinical trial is hopeless when one or more of the following are met:
(1) value of the mTR is less than −0.3 and the theta estimation is negative;
(2) the number of observed negative theta estimation (count each pair) is bigger than 90;
(3) value of the score statistics is constantly trending down or are constant negative along information time;
(4) the slope of a plot of Score Statistics vs information time is zero or near zero and there is no or very limited chance to cross the success boundary; and
(5) a new sample size is more than 3 folds of the sample size as planned.

In one embodiment, when the clinical trial is promising, the method further comprises conducting an evaluation of the clinical trial, and outputting a second result indicating whether a sample size adjustment is needed. In one embodiment, when SSR is stabilized within [0.6-1.2], no sample size adjustment is needed. In one embodiment, when SSR is stabilized and less than 0.6 or high than 1.2, the sample size adjustment is needed, wherein a new sample size is calculated by satisfying:

$$\hat{θ} \sqrt{{I}_{{N}_{n e w}} - {i}_{{n}_{E} , {n}_{C}}} \geq \frac{( C \sqrt{{I}_{N}} - {s}_{{n}_{E} , {n}_{C}} )}{\sqrt{{I}_{N} - {i}_{{n}_{E} , {n}_{C}}}} + {Φ}^{- 1} ( 1 - β ) , or$$

$${I}_{{N}_{n e w}} \geq {( \hat{θ} )}^{- 2} {( ( C \sqrt{{I}_{N}} - {s}_{{n}_{E} , {n}_{C}} ) / \sqrt{{I}_{N} - {i}_{{n}_{E} , {n}_{C}}} + {Φ}^{- 1} ( 1 - β ) )}^{2} + {i}_{{n}_{E} , {n}_{C}} ,$$

wherein (1−β) is a desired conditional power.

In one embodiment, the data collection system is an Electronic Data Capture (EDC) System. In one embodiment, the data collection system is an Interactive Web Respond System (IWRS). In one embodiment, the engine is a Dynamic Data Monitoring (DDM) engine. In one embodiment, the desired conditional power is at least 90%.

In one embodiment, the present invention provides a system for dynamically monitoring and evaluating an on-going clinical trial associated with a disease or condition, the system comprising:

1) a data collection system that collects blinded data from the clinical trial in real time,
2) an unblinding system, operable with the data collection system, that automatically unblind the blinded data into unblinded data,
3) an engine that continuously calculates statistical quantities, threshold values and success and failure boundaries based on the unblinded data, and
4) an outputting unit or graphical user interface that outputs an evaluation result indicating one of the following:
   the clinical trial is promising; and
   the clinical trial is hopeless and should be terminated;
wherein statistical quantities are selected from one or more from Score statistics, point estimate (\hat{θ}) and its 95% confidence interval, Wald statistics (Z(t)), conditional power (CP(θ,N,C|μ)), maximum trend ratio (mTR), sample size ratio (SSR), and mean trend ratio.

In one embodiment, the clinical trial is promising when one or more of the following are met:
(1) value of the score statistics is constantly trending up or are constant positive along information time,
(2) the slope of a plot of Score Statistics vs information time is positive,
(3) value of maximum Trend Ratio (mTR) is in a range of in (0.2, 0.4),
(4) value of mean trend ratio is no less than 0.2, and
(5) a new sample size is no more than 3 folds of the sample size as planned.

In one embodiment, the clinical trial is hopeless when one or more of the following are met:
(1) value of the mTR is less than −0.3 and the theta estimation is negative,
(2) the number of observed negative theta estimation (count each pair) is bigger than 90,
(3) value of the score statistics is constantly trending down or are constant negative along information time,
(4) the slope of a plot of Score Statistics vs information time is zero or near zero and there is no or very limited chance to cross the success boundary, and
(5) a new sample size is more than 3 folds of the sample size as planned.

In one embodiment, when the clinical trial is promising, the engine further conducts an evaluation of the clinical trial, and outputs a second result indicating whether a sample size adjustment is needed. In one embodiment, when SSR is stabilized within [0.6-1.2], no sample size adjustment is needed. In one embodiment, when SSR is stabilized and less than 0.6 or high than 1.2, the sample size adjustment is needed, wherein a new sample size is calculated by satisfying:

$$\hat{θ} \sqrt{{I}_{{N}_{n e w}} - {i}_{{n}_{E} , {n}_{C}}} \geq \frac{( C \sqrt{{I}_{N}} - {s}_{{n}_{E} , {n}_{C}} )}{\sqrt{{I}_{N} - {i}_{{n}_{E} , {n}_{C}}}} + {Φ}^{- 1} ( 1 - β ) , or$$

$${I}_{{N}_{n e w}} \geq {( \hat{θ} )}^{- 2} {( ( C \sqrt{{I}_{N}} - {s}_{{n}_{E} , {n}_{C}} ) / \sqrt{{I}_{N} - {i}_{{n}_{E} , {n}_{C}}} + {Φ}^{- 1} ( 1 - β ) )}^{2} + {i}_{{n}_{E} , {n}_{C}} ,$$

wherein (1−β) is a desired conditional power.

In one embodiment, the data collection system is an Electronic Data Capture (EDC) System. In one embodiment, the data collection system is an Interactive Web Respond System (IWRS). In one embodiment, the engine is a Dynamic Data Monitoring (DDM) engine. In one embodiment, the desired conditional power is at least 90%.

Although this invention has been described with a certain degree of particularity, it is to be understood that the present disclosure has been made only by way of illustration and that numerous changes in the details of construction and arrangement of parts may be resorted to without departing from the spirit and the scope of the invention.

In one embodiment, the present invention discloses a graphical user interface-based system for dynamically monitoring and evaluating an on-going clinical trial associated with a disease or condition. In one embodiment, the system comprises:
(1) a data collection system that dynamically collects blinded data from said on-going clinical trial in real time,
(2) an unblinding system, operable with said data collection system, that automatically unblinds said blinded data into unblinded data,
(3) an engine that continuously calculates statistical quantities, threshold values and success and failure boundaries based on said unblinded data and exports to a graphical user interface (GUI), and
(4) an outputting unit that dynamically outputs to said GUI a first evaluation result indicating one of the following:
   said on-going clinical trial is promising; and
   said on-going clinical trial is hopeless and should be terminated;
wherein said GUI comprises a menu allowing a user to select one or more said statistical quantities selected from the group consisting of maximum trend ratio (mTR), sample size ratio (SSR), and mean trend ratio,
wherein:
said mTR=max TR(l), wherein

$$T R ( l ) = E ( \frac{1}{ι} {\sum }_{i = 0}^{l - 1} sign ( S ( {t}_{i + 1} ) - S ( {t}_{i} ) ) ) ,$$

t=iₙ_{_{E}}_{,n}_{_{C}}/I_{N}_{₀} as the information time (fraction) based on the originally planned information I_{N}_{₀} at any iₙ_{_{E}}_{,n}_{_{C}};
said SSR=new sample size/original sample size, wherein said new sample size (I_{N}_{_{new}}) is calculated by satisfying:

$$\hat{θ} \sqrt{{I}_{{N}_{new}} - {i}_{{n}_{E} , {n}_{C}}} \geq \frac{( c \sqrt{{I}_{N}} - {S}_{{n}_{E} , {n}_{C}} )}{\sqrt{{I}_{N} - {i}_{{n}_{E} , {n}_{C}}}} + {Φ}^{- 1} ( 1 - β ) ,$$

or, equivalently,
I_{N}_{_{new}}≥(\hat{θ})⁻²((C\sqrt{I_{N}}−Sₙ_{_{E}}_{,n}_{_{C}})/{square root over (I_{N}−iₙ_{_{E}}_{,n}_{_{C}})}+Φ⁻¹(1−β))²+iₙ_{_{E}}_{,n}_{_{C}}, wherein (1−β) is a desired conditional power; and
said mean trend ratio is calculated by:

$$\frac{1}{i - A + 1} ( {\sum }_{j = A}^{l} TR ( j ) ) = \frac{1}{i - A + 1} ( {\sum }_{j = A}^{l} \frac{1}{j} {\sum }_{i = 0}^{j - 1} sign ( S ( {t}_{i + 1} ) - S ( {t}_{i} ) ) ) ,$$

wherein l represents the l^{th} block of patients to be monitored, and A is the 1^{st} block to start monitoring.

In one embodiment, the statistical quantities further comprise one or more of Score statistics, point estimate (\hat{θ}) and its 95% confidence interval, Wald statistics (Z(t)), and conditional power (CP(θ,t,C|u)) calculated by

$$CP ( θ , N , C \vert u ) = P ( \frac{{S}_{n}}{\sqrt{{I}_{N}}} \geq C \vert {S}_{{n}_{E} , {n}_{C}} = u ) = 1 - Φ ( \frac{c \sqrt{{I}_{N}} - u - θ ( {I}_{N} - {i}_{{n}_{E} , {n}_{C}} )}{\sqrt{{I}_{N} - {i}_{{n}_{E} , {n}_{C}}}} ) ,$$

wherein Φ is the standard normal distribution function.

In one embodiment, the GUI reveals via a subsection thereof that said on-going clinical trial is promising, when one or more of the following are met:
(1) value of the Score statistics is constantly trending up or is constantly positive along information time,
(2) the slope of a plot of the Score statistics versus information time is positive,
(3) value of said mTR is in the range of (0.2, 0.4),
(4) value of said mean trend ratio is no less than 0.2, and
(5) said sample size ratio (SSR) is no more than 3.

In one embodiment, the GUI reveals via a subsection thereof that said on-going clinical trial is hopeless and should be terminated, when one or more of the following are met:
(1) value of said mTR is less than −0.3, and said point estimate is negative,
(2) said point estimate is observed to be negative for over 90 times (count each pair),
(3) value of said Score statistics is constantly trending down or is constantly negative along information time,
(4) the slope of a plot of said Score statistics versus information time is zero or near zero, and there is no or very limited chance for said Score statistics to cross said success boundary with a statistically significant level p<0.05, and
(5) said sample size ratio (SSR) is greater than 3.

In one embodiment, when said on-going clinical trial is promising, said engine further conducts a second evaluation of said on-going clinical trial and outputs to said GUI a second result indicating whether a sample size adjustment is needed.

In one embodiment, the GUI reveals that no sample size adjustment is needed when said SSR is stabilized in the range of [0.6, 1.2].

In one embodiment, the GUI reveals that a sample size adjustment is needed when said SSR is stabilized and less than 0.6 or greater than 1.2.

In one embodiment, the data collection system is an Electronic Data Capture (EDC) System or Interactive Web Respond System (IWRS).

In one embodiment, the engine is a Dynamic Data Monitoring (DDM) engine.

In one embodiment, the desired conditional power is at least 90%.

In one embodiment, the present invention discloses a graphical user interface-based method of dynamically monitoring and evaluating an on-going clinical trial associated with a disease or condition. In one embodiment, the method comprises:
(1) dynamically collecting blinded data by a data collection system from said on-going clinical trial,
(2) automatically unblinding said blinded data by an unblinding system operable with said data collection system into unblinded data,
(3) continuously calculating statistical quantities, threshold values, and success and failure boundaries by an engine based on said unblinded data, wherein said statistical quantities, threshold values, and success and failure boundaries are communicated to a graphical user interface (GUI), and
(4) dynamically outputting to said GUI a first evaluation result indicating one of the following:
   said on-going clinical trial is promising, and
   said on-going clinical trial is hopeless and should be terminated,
wherein said GUI comprises a menu allowing a user to select one or more of said statistical quantities selected from the group consisting of maximum trend ratio (mTR), sample size ratio (SSR), and mean trend ratio,
wherein:
said

$$mTR = {max}_{1} TR ( l ) ,$$

wherein

$$TR ( l ) = E ( \frac{1}{l} {\sum }_{i = 0}^{l - 1} sign ( S ( {t}_{i + 1} ) - S ( {t}_{i} ) ) ) ,$$

t=iₙ_{_{E}}_{,n}_{_{C}}/I_{N}_{₀} as the information time (fraction) based on the originally planned information I_{N}_{₀} at any iₙ_{_{E}}_{,n}_{_{C}}; said SSR=new sample size/original sample size, wherein said new sample size (I_{N}_{_{new}}) is calculated by satisfying:

$$\hat{θ} \sqrt{{I}_{{N}_{new}} - {i}_{{n}_{E} , {n}_{C}}} \geq \frac{( c \sqrt{{I}_{N}} - {S}_{{n}_{E} , {n}_{C}} )}{\sqrt{{I}_{N} - {i}_{{n}_{E} , {n}_{C}}}} + {Φ}^{- 1} ( 1 - β ) ,$$

or, equivalently,
I_{N}_{_{new}}≥(\hat{θ})⁻²((\sqrt{I_{N}}−Sₙ_{_{E}}_{,n}_{_{C}})/{square root over (I_{N}−iₙ_{_{E}}_{,n}_{_{C}})}+Φ⁻¹(1−β))²+iₙ_{_{E}}_{,n}_{_{C}}, wherein (1−β) is a desired conditional power; and
said mean trend ratio is calculated by:

$$\frac{1}{i - A + 1} ( {\sum }_{j = A}^{l} TR ( j ) ) = \frac{1}{i - A + 1} ( {\sum }_{j = A}^{l} \frac{1}{j} {\sum }_{i = 0}^{j - 1} sign ( S ( {t}_{i + 1} ) - S ( {t}_{i} ) ) ) ,$$

wherein l represents the block of patients to be monitored, and A is the 1^{st} block to start monitoring.

In one embodiment, the statistical quantities further comprise one or more of Score statistics, point estimate (\hat{θ}) and its 95% confidence interval, Wald statistics (Z(t)), and conditional power (CP(θ,t,C|u)) calculated by

$$CP ( θ , N , C \vert u ) = P ( \frac{{S}_{n}}{\sqrt{{I}_{N}}} \geq C \vert {S}_{{n}_{E} , {n}_{C}} = u ) = 1 - Φ ( \frac{c \sqrt{{I}_{N}} - u - θ ( {I}_{N} - {i}_{{n}_{E} , {n}_{C}} )}{\sqrt{{I}_{N} - {i}_{{n}_{E} , {n}_{C}}}} ) ,$$

wherein Φ is the standard normal distribution function.

In one embodiment, the GUI reveals that said on-going clinical trial is promising, when one or more of the following are met:
(1) value of said mTR is in the range of (0.2, 0.4),
(2) value of said mean trend ratio is no less than 0.2,
(3) value of said Score statistics is constantly trending up or is constantly positive along information time,
(4) the slope of a plot of said Score statistics versus information time is positive, and
(5) said sample size ratio (SSR) is no more than 3.

In one embodiment, the GUI reveals that said on-going clinical trial is hopeless and should be terminated, when one or more of the following are met:
(1) value of said mTR is less than −0.3, and said point estimate is negative;
(2) said point estimate is observed to be negative for over 90 times (count each pair);
(3) value of said Score statistics is constantly trending down or is constantly negative along information time;
(4) the slope of a plot of said Score statistics versus information time is zero or nearly zero, and there is no or very limited chance for said Score statistics to cross said success boundary with a statistically significant level p<0.05; and
(5) said sample size ratio (SSR) is greater than 3.

In one embodiment, when said on-going clinical trial is promising, said method further comprises conducting a second evaluation of said on-going clinical trial and outputting to said GUI a second result indicating whether a sample size adjustment is needed.

In one embodiment, the GUI reveals that no sample size adjustment is needed when said SSR is stabilized in the range of [0.6, 1.2].

In one embodiment, the GUI reveals that a sample size adjustment is needed when said SSR is stabilized and less than 0.6 or greater than 1.2.

In one embodiment, the data collection system is an Electronic Data Capture (EDC) System, or Interactive Web Respond System (IWRS).

In one embodiment, the engine is a Dynamic Data Monitoring (DDM) engine.

In one embodiment, the desired conditional power is at least 90%.

The invention will be better understood by reference to the Experimental Details which follow, but those skilled in the art will readily appreciate that the specific experiments detailed are only illustrative, and are not meant to limit the invention as described herein, which is defined by the claims following thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended, and does not exclude additional, un-recited elements or method steps.

### EXAMPLES

### Example 1

### The Initial Design

In general, let θ denote the treatment effect size, which may be the difference in means, log-odds ratio, log-hazards ratio, etc. as dictated by the type of endpoint being studied. The design specifies a planned/initial sample size (or “information” in general) N₀ per arm, with a type-I error rate of a, and certain desired power, to test the null hypothesis H₀: θ=0 versus H_{A}: θ>0. For simplicity, two treatment groups with equal randomization are considered with an assumption that that the primary endpoint is normally distributed. Let X_{E}˜N (μ_{E},σ_{E}²) and X_{C}˜N (μ_{C},σ_{C}²) be the efficacy endpoints for the experimental and control groups, respectively. θ=μ_{E}−μ_{C}. For other endpoints, similar statistics (such as the score function, z-score, information time, etc.) can be constructed using normal approximations.

### Occasional and Continuous Monitoring

Some key statistics are laid out in this section. The AGSD currently in common practice provides occasional data monitoring. DAD/DDM can monitoring the trial and examine the data after each patient entry. The possible actions of data monitoring include: to continue accumulating the trial data without modification, to raise a signal to perform formal interim analysis, which may be of either futility or early efficacy, or to consider a sample size adjustment. The basic set-up of the initial trial design and mathematical notation for data monitoring are similar between the two. The present invention discloses how to find a proper time-point to perform a just-in-time formal interim analysis with DAD/DDM. Prior to this time-point, trial is continuing without modification. The alpha-spending function approach for continuous or occasional monitoring data of Lan, Rosenberger and Lachin (1993) is very flexible regarding testing the hypothesis at any information time. However, the timing for sample size adjustment—specifically, increase of sample size, is not a simple matter. A stable estimate of the effect size is needed to determine the increment, and presumably, the decision of increasing sample size should be made only once during the entire trial period. The following table 1 shows the timing issue with a focus on sample size re-estimate (SSR). For the first scenario in Table 1, the true value and assume value of 0 are 0.2 and 0.4, respectively. The initial sample size based on the assumed value is 133, which is much less than the one based on true value (i.e., 526). If the SSR is conducted at a time pre-fixed at 50% (67 patients), the adjustment is too early. For the second scenario in Table 1, the timing for SSR is conducted at 50% (263 patients), which is too late.

TABLE 1
Timing to conduct sample size re-estimation (SSR)
(Assumption: 90% power and σ = 1)

| | SS based on | | SS based on | 50% of | |
|---|---|---|---|---|---|
| True θ | true θ | Assumed θ | assumed θ | planned | Comment |
| 0.2 | 526 | 0.4 | 133 | 67 | Too early |
| 0.4 | 133 | 0.2 | 526 | 263 | Too late |

At an arbitrary time-point expressed by the number of subjects in the experimental group (n_{E}) and in the control arm (n_{C}), the sample means are

$${\overline{X}}_{E , {n}_{E}} = \frac{1}{{n}_{E}} {\sum }_{i = 1}^{{n}_{E}} {X}_{E , i} \sim N ( {μ}_{E} , \frac{{σ}_{E}^{2}}{{n}_{E}} ) and$$

$${\overline{X}}_{C , {n}_{C}} = \frac{1}{{n}_{C}} {\sum }_{i = 1}^{{n}_{C}} {X}_{C , i} \sim N ( {μ}_{C} , \frac{{σ}_{C}^{2}}{{n}_{C}} ) .$$

θ=X̅_{E,n}_{_{E}}−X̅_{C,n}_{_{C}}. The Wald statistics is

$${Z}_{{n}_{E} , {n}_{C}} = ( {\overline{X}}_{E , {n}_{E}} - {\overline{X}}_{C , {n}_{C}} ) / \sqrt{\frac{{\hat{σ}}_{E}^{2}}{{n}_{E}} + \frac{{\hat{σ}}_{C}^{2}}{{n}_{C}}} ,$$

where \hat{σ}_{E}² and \hat{σ}_{C}², are the estimated variances for X_{E} and X_{C}, respectively. The estimated Fisher's information is

$${i}_{{n}_{E} , {n}_{C}} = {( \frac{{\hat{σ}}_{E}^{2}}{{n}_{E}} + \frac{{\hat{σ}}_{C}^{2}}{{n}_{C}} )}^{- 1} .$$

Let the score function be

$$S ( {i}_{{n}_{E} , {n}_{C}} ) = {S}_{{n}_{E} , {n}_{C}} = {Z}_{{n}_{E} , {n}_{C}} / \sqrt{\frac{{\hat{σ}}_{E}^{2}}{{n}_{E}} + \frac{{\hat{σ}}_{C}^{2}}{{n}_{C}}} = {Z}_{{n}_{E} , {n}_{C}} \sqrt{{i}_{{n}_{E} , {n}_{C}}} = \hat{θ} {i}_{{n}_{E} , {n}_{C}} .$$

Sₙ_{_{E}}_{,n}_{_{C}}˜N(θiₙ_{_{E}}_{,n}_{_{C}},iₙ_{_{E}}_{,n}_{_{C}}).

At the end of the trial, I_{N}=N(\hat{σ}_{E}²+\hat{σ}_{C}²)⁻¹ per group, where N=N₀ if no change of the planned sample size or N=N_{new}; see Eq. (2) below. S_{N}=S_{N,N}˜N(θI_{N},I_{N}). Under the null hypothesis, approximately, S_{N}˜N(0, I_{N}) and

$${Z}_{N} = \frac{{S}_{N}}{\sqrt{{I}_{N}}} \sim N ( 0 , 1 ) .$$

The null hypothesis is rejected if

$$\frac{{S}_{N}}{\sqrt{{I}_{N}}} \geq C .$$

The cut-off C is chosen so that the type-I error rate is preserved at α, taking into account of possible multiplicity in testing such as sequential tests, SSR, and multiple endpoints. Details will be given in the sequel.

Given Sₙ_{_{E}}_{,n}_{_{C}}=sₙ_{_{E}}_{,n}_{_{C}}=u at iₙ_{_{E}}_{,n}_{_{C}}, S_{N}−Sₙ_{_{E}}_{,n}_{_{C}}˜N(θ[I_{N}−Iₙ_{_{E}}_{,n}_{_{C}}],[I_{N}−iₙ_{_{E}}_{,n}_{_{C}}]). The conditional power CP(θ,N,C|Sₙ_{_{E}}_{,n}_{_{C}}) is

$$CP ( θ , N , C \vert u ) = P ( \frac{{S}_{n}}{\sqrt{{I}_{N}}} \geq C \vert {S}_{{n}_{E} , {n}_{C}} = u ) = 1 - Φ ( \frac{c \sqrt{{I}_{N}} - u - θ ( {I}_{N} - {i}_{{n}_{E} , {n}_{C}} )}{\sqrt{{I}_{N} - {i}_{{n}_{E} , {n}_{C}}}} ) , \qquad ( 1 )$$

The conditional power (1) for given N and C is conditioning on two quantities: the unknown treatment effect size θ and the observed Sₙ_{_{E}}_{,n}_{_{C}}=sₙ_{_{E}}_{,n}_{_{C}}. Value of θ can be based on several considerations and is up to the choice of the researcher, including, for example, the optimistic estimate, which is the specific value in H_{A} on which the original sample size/power was based, the pessimistic estimate, which is 0 under H₀, the point estimate \hat{θ}, or some confidence limits based on \hat{θ}, or some combination of the above, perhaps even with other external information or opinion of a clinical meaningful effect that needs to be detected. A predictive power is obtained upon averaging Eq. (1) over a prior distribution of θ. These options are offered in the DAD/DDM procedure. In AGSD, a common (default) choice for calculating the new sample size is to use simply the point estimate \hat{θ} in (1), i.e., assuming the current observed trend will continue. The new sample size (information) to meet the desired conditional power of 1−β should satisfy

$$\hat{θ} \sqrt{{I}_{{N}_{new}} - {i}_{{n}_{E} , {n}_{C}}} \geq \frac{( C \sqrt{{I}_{N}} - {S}_{{n}_{E} , {n}_{C}} )}{\sqrt{{I}_{N} - {i}_{{n}_{E} , {n}_{C}}}} + {Φ}^{- 1} ( 1 - β ) , or {I}_{{N}_{new}} \geq {( \hat{θ} )}^{- 2} {( ( C \sqrt{{I}_{N}} - {S}_{{n}_{E} , {n}_{C}} ) / \sqrt{{I}_{N} - {i}_{{n}_{E} , {n}_{C}}} + {Φ}^{- 1} ( 1 - β ) )}^{2} + {i}_{{n}_{E} , {n}_{C}} . \qquad ( 2 )$$

Let

$$r = \frac{{I}_{{N}_{new}}}{{I}_{{N}_{0}}} .$$

Thus, r >1 suggests a need for sample size increase, and r<1 suggests sample size reduction. Note that

$$\hat{θ} = \frac{{S}_{{n}_{E} , {n}_{C}}}{{i}_{{n}_{E} , {n}_{C}}} ∼ N ( θ , \frac{1}{{i}_{{n}_{E} , {n}_{C}}} ) .$$

Moreover, although using conditional power to re-estimate the sample size is quite rational, it is not the only consideration for sample size adjustment. In practice, there may be budgetary concerns that would cap the sample size adjustment, or regulatory reasons to whole-number the new sample size to avoid a possible “back-calculation” that could reveal the exact \hat{θ}. These restrictions would of course affect the resulting conditional power. It is also often for a “pure” SSR not to reduce the planned sample size (i.e., not allow r<1) to avoid confusion with early stop procedures (for futility or efficacy). Later when futility with SSR is considered, sample size reduction will be allowed. See Shih, Li and Wang (2016) for more discussion on calculating I_{N}_{_{new}}.

To control the type-I error rate, the critical/boundary value C is considered as follows.

Without any interim analysis for efficacy, if there is no change of the planned information time I_{N}_{₀}, then the null hypothesis is rejected if

$$\frac{S ( {I}_{{N}_{0}} )}{\sqrt{{I}_{{N}_{0}}}} \geq {Z}_{1 - α} = {C}_{0} .$$

(For one-sided test, α=0.025, C₀=1.96). With the change to I_{N}_{_{new}}, to preserve the type-I error rate, the final critical boundary C₀ must be adjusted to C₁, which satisfies P(S(I_{N}_{_{new}}.)≥C₁{square root over (I_{N}_{_{new}})}·|S(iₙ_{_{E}}_{,n}_{_{C}})=u)=P(S(I_{N}_{₀})≥C₀\sqrt{I_{N}_{₀}}|S(iₙ_{_{E}}_{,n}_{_{C}})=u), using the independent increment property of the partial sum process of the score function (which is a Brownian motion). Thus C₁ is solved as (Gao, Ware and Mehta (2008)):

$${C}_{1} = \frac{1}{\sqrt{{I}_{{N}_{n e w}}}} \{ \frac{\sqrt{{I}_{{N}_{new}} - {i}_{{n}_{E} , {n}_{C}}}}{\sqrt{{I}_{{N}_{0}} - {i}_{{n}_{E} , {n}_{C}}}} ( {C}_{0} \sqrt{{I}_{{N}_{0}}} - u ) \} + \frac{u}{\sqrt{{I}_{{N}_{n e w}}}} . \qquad ( 3 )$$

That is, without any interim analysis for early efficacy, the null hypothesis will be rejected if

$$\frac{S ( {I}_{{N}_{n e w}} )}{\sqrt{{I}_{{N}_{new}}}} \geq {C}_{1}$$

after SSR at iₙ_{_{E}}_{,n}_{_{C}}, where C₁ satisfies Eq. (3). That is, C=C₁ in Eq. (1). Notice, C₁=C₀ if N_{new}=N₀.

If prior to SSR a GS boundary is employed for early efficacy monitoring, and the final boundary value is C_{g}, then C₀ in (3) should be replaced by C_{g}. C_{g} in DAD/DDM with continuous monitoring that permitting early stop for efficacy is discussed in Example 3. For example, with one-sided test where α=0.025, C₀=1.96 (without interim efficacy analysis), C_{g}=2.24 (with O'Brien-Fleming boundary).

Note that Chen, DeMets and Lan (2004) showed that if CP(\hat{θ}, N₀, C|Sₙ_{_{E}}_{,n}_{_{C}}) the conditional power for the planned end time using the current point estimate of θ at iₙ_{_{E}}_{,n}_{_{C}} is at least 50%, then increasing sample size will not inflate the type-I error, hence there is no need to change the final boundary C₀ (or C_{g}) to C₁ for the final test.

### Accumulating Data in DAD/DDM

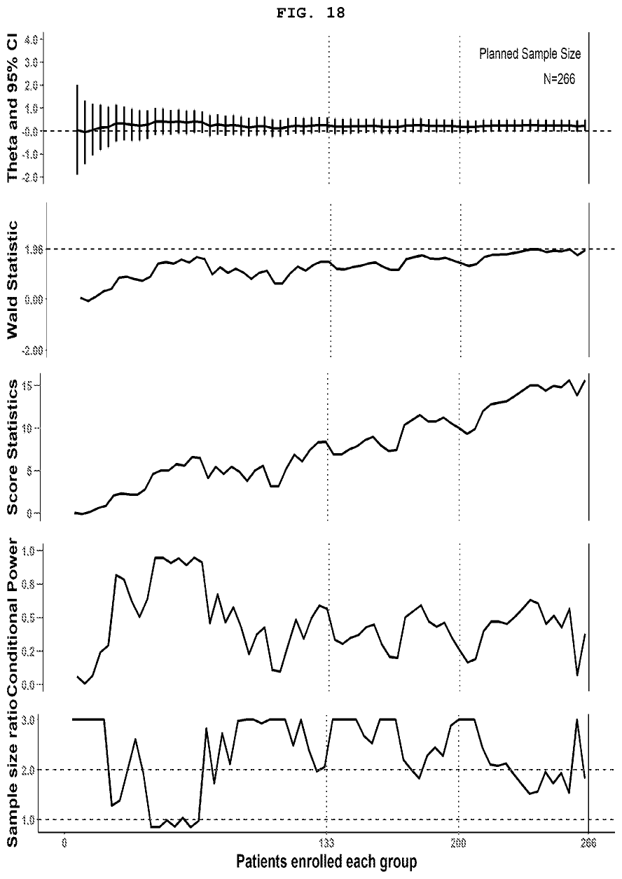

FIG. 18 illustrates the features of DAD/DMM by a simulated clinical trial with true θ=0.25 with common variance 1. Here, a sample size of N=336 per arm is needed with 90% power at α=0.025 (one-sided). However, it is assumed that θ_{assumed}=0.4 in planning the study and the planned sample size of N₀=133 per arm is used (266 in total). The trial is monitored continuously after each patient entry. The point estimate of

$$\hat{θ} = \frac{{S}_{{n}_{E} , {n}_{C}}}{{i}_{{n}_{E} , {n}_{C}}}$$

and 95% confidence interval, the Wald statistic (z-score, Zₙ_{_{E}}_{,n}_{_{C}}), the score function, the conditional power CP(\hat{θ},N₀,C|Sₙ_{_{E}}_{,n}_{_{C}}) and the information ratio

$$r = \frac{{I}_{{N}_{new}}}{{I}_{{N}_{0}}}$$

are plotted along the patients enrolled (n_{E}+n_{C}=n) axis for C=1.96. The following are observed:
1) All the curves fluctuate at both 50% (n=133) and 75% (n=200) of enrollment, commonly used time-points for interim analyses.
2) The point estimate

$$\hat{θ} = \frac{{S}_{{n}_{E} , {n}_{C}}}{{i}_{{n}_{E} , {n}_{C}}}$$

stabilizes to the positive direction, indicating positive efficacy.
3) The Wald statistics Zₙ_{_{E}}_{,n}_{_{C}} trends upward and close to but is unlikely to cross the critical value C=1.96 at the planned sample size of N₀=133 per arm. That is, the trial is promising and a sample size increase could help to make it eventually successful.
4) The

$$ratio = \frac{{I}_{{N}_{new}}}{{I}_{{N}_{0}}}$$

is above 2, suggesting that the sample size needs to be at least doubled.
5) The conditional power curve approaches zero in this setting since Zₙ_{_{E}}_{,n}_{_{C}} approaches somewhere below C=1.96. (See discussion in Example 2)

In this simulated example, the continuous data monitoring provides a better understanding of the behavior of data as the trial progresses. By analyzing the accumulative data, whether a trial is promising or hopeless can be detected. If it deems to be a hopeless trial, sponsor can make a “No Go” decision and terminate it earlier to avoid unethical patient suffering and financial waste. In one embodiment, SSR as disclosed in the present invention could make a promising trial eventually successful. Furthermore, even though a clinical trial is started with a wrong guess of treatment effect (θ_{assumed}), the data-guided analysis will lead a promising trial to the right target with an updated design, e.g., a corrected sample size. Example 2 below will show a trend ratio method as a tool to assess whether a trial is promising by using DAD/DDM. The trend ratio and futility stopping rules that are also disclosed herein can further help the decision making.

### Example 2

DAD/DDM with Consideration of SSR: Timing the SSR

Conditional power is useful in calculating I_{N}_{_{new}}, but not so useful in properly timing the interim analysis for SSR. By replacing sₙ_{_{E}}_{,n}_{_{C}}=Zₙ_{_{E}}_{,n}_{_{C}}\sqrt{iₙ^{E},n^{C}} in Eq. (1), as iₙ_{_{E}}_{,n}_{_{C}} approaches I_{N}_{₀}, i.e., as the enrollment increases to the planned sample size, there are only two possibilities for the conditional power: it either approaches to zero (when Zₙ_{_{E}}_{,n}_{_{C}} approaches somewhere below C), or to 1 (when Zₙ_{_{E}}_{,n}_{_{C}} approaches somewhere above C). For timing the SSR, the stability of \hat{θ} is also investigated. Since

$$\hat{θ} = \frac{{S}_{{n}_{E} , {n}_{C}}}{{i}_{{n}_{E} , {n}_{C}}} \sim N ( θ , \frac{1}{{i}_{{n}_{E} , {n}_{C}}} ) ,$$

it stabilizes when iₙ_{_{E}}_{,n}_{_{C}} increases. The additional information beyond the current observation Sₙ_{_{E}}_{,n}_{_{C}} at iₙ_{_{E}}_{,n}_{_{C}} that can provide desired power for the trial is I_{N}_{_{new}}−iₙ_{_{E}}_{,n}_{_{C}}, which also becomes more stable (thus more reliable) as iₙ_{_{E}}_{,n}_{_{C}} increases. However, if an adjustment is necessary, the later SSR is performed, the less interest and feasibility there is operationally to adjust sample size. Since it is difficult to make ‘operation interest and feasibility’ a quantifiable objective function or a constraint, as needed for any optimization problem, the present invention opts to using some trend stabilization method as follows.

### Trend Ratio and Maximum Trend Ratio

In this section, the present invention discloses a tool for trend analysis using DAD/DDM to assess whether the trial is trending for success (i.e., whether the trial is promising). This tool uses characteristics of Brownian motions that reflect the trend of the trajectory. Toward this end, denote t=iₙ_{_{E}}_{,n}_{_{C}}/I_{N}_{₀} as the information time (fraction) based on the originally planned information I_{N}_{₀} at any iₙ_{_{E}}_{,n}_{_{C}}. Let S(t)≈B(t)+θt˜N(θt,t) be the score function at information time t, where B(t)˜N(0, t) is the standard continuous Brownian motion process (see, e.g., Jennison and Turnbull (1997)).

Under the alternative hypothesis of θ>0, the mean trajectory of S(t) is upwards and the curve should hover around the line y(t)=Bt. If the curve at discrete information time t₁, t₂, . . . is inspected, then more line segments S(tᵢ₊ᵢ) −S(tᵢ) should be upwards (i.e., sign(S(tᵢ₊₁) −S(tᵢ))=1) than those that are downwards (i.e., sign(S(tᵢ₊₁) −S(tᵢ))=−1). Let l be the total of the number of line segments examined, then the expected “trend ratio” of length l, TR(l), is

$$E ( \frac{1}{l} {\sum }_{i = 0}^{l - 1} sign ( S ( {t}_{i + 1} ) - S ( {t}_{i} ) ) ) > 0 .$$

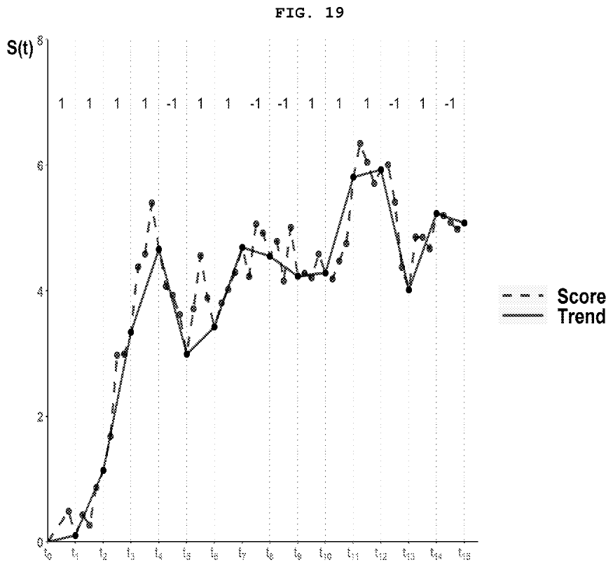

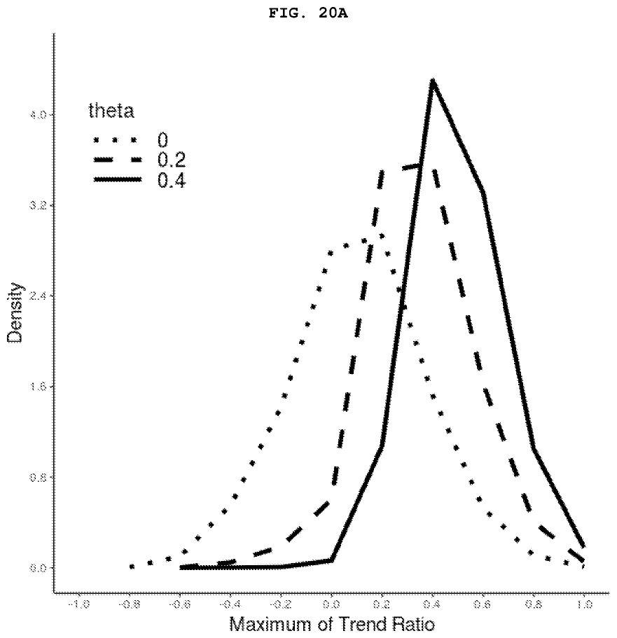

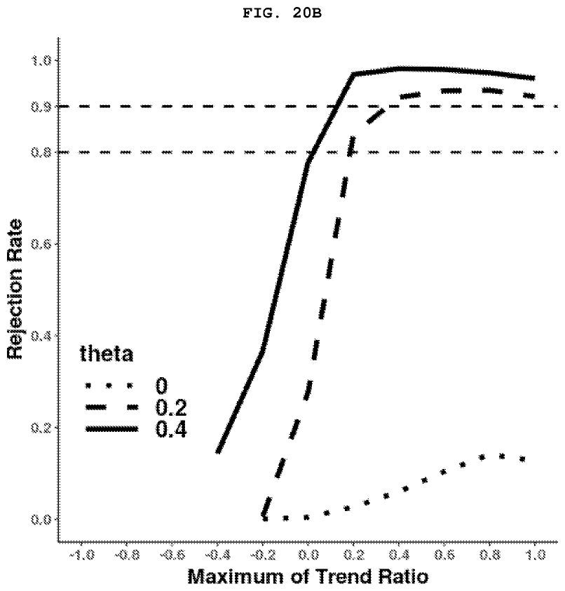

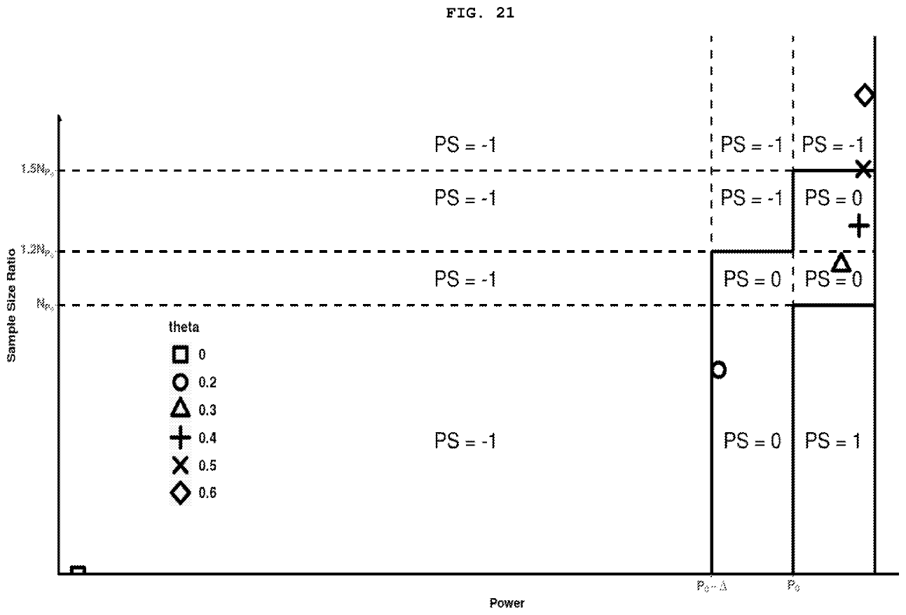

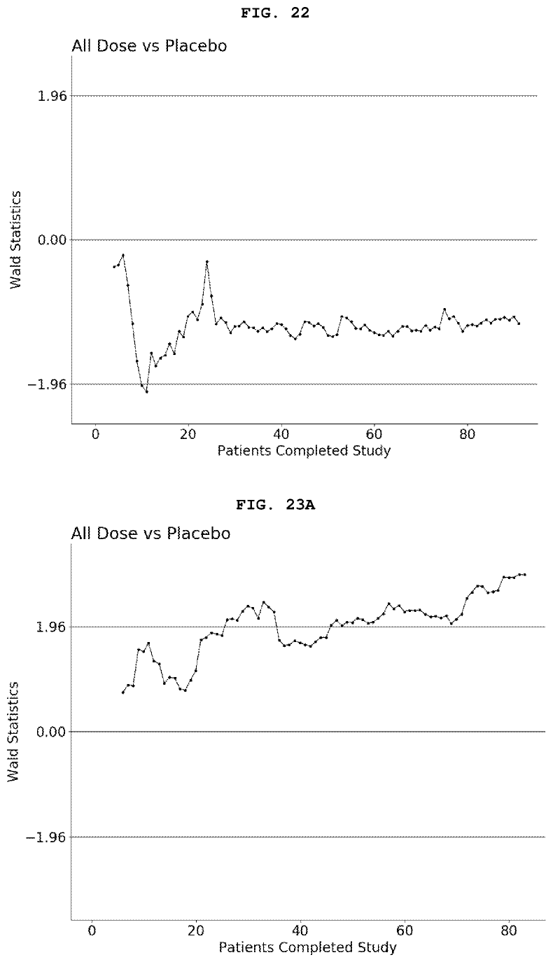

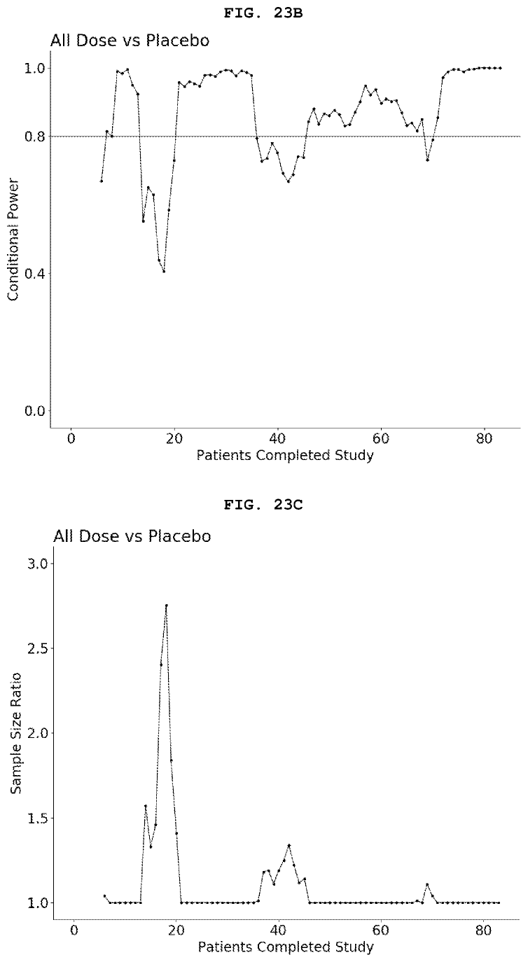

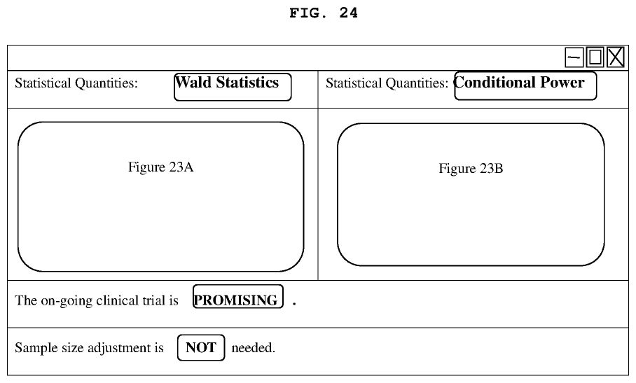

This trend ratio is similar to the “moving average” in time series analysis of financial data. The present invention equally spaces the time information times tᵢ,tᵢ₊₁, tᵢ₊₂, . . . , according to the block size used by the original randomization (e.g., every 4 patients as demonstrated here) and start the trend ratio calculation when l is, say ≥10 (i.e., with at least 40 patients total). Here the starting time-point and the block size in terms of number of patients are options for DAD/MDD. FIG. 19 illustrates a trend ratio calculation according to one embodiment of the present invention.

In FIG. 19, the trend sign(S(tᵢ₊₁)−S(tᵢ)) is calculated for every 4 patients (between tᵢ₊₁ and tᵢ) and start calculating the TR(l) when l≥10. When there are 60 patients at t₁₂, TR(l) for l=10, 11, are calculated. The maximum of the 6 TRs in FIG. 19 is equal to 0.5 (when 1=12). The maximum TR (mTR) would conceivably be more sensitive than the mean trend ratio to pick up the trend of the data of the 60 patients. The mTR=0.5 indicates a positive trend during the segments being examined.

To study the property and possible use of mTR, a simulation study with 100,000 runs was conducted for each of the 3 scenarios: θ=0, 0.2, and 0.4. In each scenario, the planned sample size is 266 in total, the trend sign(S(tᵢ₊₁)−S(tᵢ)) is calculated for every block of 4 patients between tᵢ₊₁ and tᵢ and TR(l) is started when l≥10. As usually SSR is performed no later than the information fraction ¾ (i.e., 200 patients in total here), mTR is calculated over TR(l), l=10, 11, 12, . . . , 50, i.e., starting t₁₀ till t₅₀.

FIG. 20A displays the empirical distribution of the mTR among 41 segments. As seen, mTR shifts to the right as θ increases. FIG. 20B displays the simulation results of rejecting H₀:θ=0 by applying the mTR at different cutoffs. Specifically, in each scenario of θ and each simulation run, conditioning on a≤mTR <b, the final test

$$\frac{S ( {I}_{{N}_{0}} )}{\sqrt{{I}_{{N}_{0}}}} \geq {C}_{0}$$

is performed. FIG. 20B displays the empirical estimate of

$$P ( \frac{S ( {I}_{{N}_{0}} )}{\sqrt{{I}_{{N}_{0}}}} \geq {C}_{0} \vert a \leq max \{ T R ( 1 ) , 1 = 10 , 11 , 12 , \text{...} 50 \} < b ) .$$

To differentiate it from the conditional power seen in Eq. (1), this “trend ratio based conditional power” is termed as CP_{TR(N}_{₀}₎. It shows that the larger cutoff value is, the higher chance is for the trial finally in rejection region of the null hypothesis. For example, when θ=0.2 (relatively small treatment effect size compared to θ=0.4), 0.2≤mTR<0.4 is associated with a greater than 80% chance of correctly rejecting the null hypothesis at the end of the trial (i.e., conditional power=0.80), while maintaining conditional type-I error rate at a reasonably low level. As a matter of fact, the conditional type-I rate does not have a relevant interpretation. Rather, it is the unconditional type-I error rate to be controlled, as opposed to the conditional type-I error rate.

To use mTR in monitoring the signal of possible conducting SSR in a timely manner, FIG. 20B suggests to set 0.2 as the cutoff for mTR. It means that the timing of SSR with continuous monitoring is flexible; that is, at any iₙ_{_{E}}_{,n}_{_{C}}, the first time when the mTR is greater than 0.2, a new sample size is calculated. Otherwise, the clinical trial shall move on without doing SSR. In one embodiment, one can over-rule the signal, or even over-rule the new sample size calculated, move on without modification of the trial, without affecting the type-I error rate control.

With the information TR(l), l=10, 11, 12, . . . , available at iₙ_{_{E}}_{,n}_{_{C}}, when calculating the new sample size by Eq. (2), instead of using a single point estimate of

$$\hat{θ} = \frac{{S}_{{n}_{E} , {n}_{C}}}{{i}_{{n}_{E} , {n}_{C}}}$$

Sₙ_{_{E}}_{,n}_{_{C}} and iₙ_{_{E}}_{,n}_{_{C}}, the average of the \hat{θ}'s as well as the average Sₙ_{_{E}}_{,n}_{_{C}} and average iₙ_{_{E}}_{,n}_{_{C}}, are used respectively, in the interval associated with the mTR. The average Sₙ_{_{E}}_{,n}_{_{C}} and average iₙ_{_{E}}_{,n}_{_{C}} will also be applied to the calculation of the critical value C₁ in Eq. (3).

### Sample Size Ratio and Minimum Sample Size Ratio

In this section, the present invention discloses another tool for trend analysis using DAD/DDM to assess whether the trial is trending for success (i.e., whether the trial is promising).

### Comparison of SSR Using Trend to Using a Single Time-Point

The conventional SSR is usually conducted at some middle time-point when t≈½ but no later than ¾. DAD/DDM as disclosed in the present invention uses trend analysis over several time-points as described above. Both use conditional power approach, but utilize different amount of data in estimating the treatment effect. These two methods are compared by simulation as follows. Assume a clinical trial with true θ=0.25 and common variance=1. (The same set up as in the second section of Example 1). Here, a sample size of N=336 per arm (672 total) is ideally needed with 90% power at α=0.025 (one-sided). However, it is assumed that θ_{assumed}=0.4 in planning the study and the planned sample size of N=133 per arm (266 total) is used with randomization of block size 4. Two situations were compared: monitoring the trial continuously after each patient entry with the DAD/DDM procedure versus the conventional SSR procedure. Specifically, with the conventional SSR procedure, SSR at either t≈½ (N=66 per arm or 132 in total) or t≈¾ (N=100 per arm or 200 in total) was conducted using the snap-shot point estimate at these time points respectively.

With the DAD/DDM, there is no pre-specified time-point to conduct SSR, but the timing with mTR was monitored. Calculation of TR(l) started at tₗ=t₁₀ with every 4 patients entry (hence total=40 patients at t₁₀). For timing by mTR, the calculation moves along t₁₀, t₁₁, . . . t_{L} and find max of TR(l) over 1, 2, . . . L−9 segments, respectively, until the first time mTR≥0.2 or till t≈½ (132 patients in total) where t_{L}=t₃₃ and the max would be over 33−9=24 segments—to compare with the above conventional t≈½ method, or till t≈¾ (200 patients in total) where t_{L}=t₅₀ and the max would be over 50−9=41 segments—to compare with the conventional t≈¾ method. Only at the first mTR≥0.2 will the new sample size be calculated with Eq. (2) using the average of the \hat{θ}'s as well as the average Sₙ_{_{E}}_{,n}_{_{C}} and average iₙ_{_{E}}_{,n}_{_{C}} in the interval associated with mTR.

Denote i the time fraction when the SSR is conducted. For the conventional SSR method, SSR is always conducted and conducted at τ=½ or ¾ as designed. (Thus, the unconditional and conditional probabilities are the same in Table 2). For DAD/DDM, τ=(# of patients associated with the first mTR≥0.2)/266. If τ exceeds ½ (for the first comparison) or ¾ (for the second comparison), τ=1 indicates that SSR is not done. (Thus, the unconditional and conditional probabilities are different in Table 2.) The starting point for sample size change or futility are both using n>=45 while total each group is 133. The increments are both 4 pts each group.

In Table 1, sample size re-estimation is made based on “Do we have 6 consecutive sample size ratios (New sample size/original sample size) bigger than 1.02 or smaller than 0.8”. The decision is made after 45 patient each group but ratio is calculated every block (i.e. at n=4, 8, 12, 16, 20, 24, 28, 32, etc.). If all the sample size ratios at 24, 32, 36, 40, 44, 48 are bigger than 1.02 or all less than 0.8, then sample size change was made at n=48 based on the sample size re-estimation calculation at n=48. However, the present invention calculated the Max trend ratio after each simulation trial ends. It doesn't have an effect on decision of Dynamic adaptive design.

For both methods, sample size reduction (“Pure” SSR) is not allowed. If the N_{new} is less than the originally planned sample size, or the treatment effect estimate is negative, the trial shall then continue with the planned sample size (266 total). Nevertheless, SSR is conducted even though the sample size remains unchanged in these situations. Let AS=(average new sample size)/672 as the percentage of the ideal sample size under Ha, or =(average new sample size)/266 under H₀. Tables 2 and 3 show the comparisons as summarized below:
(1) When the null hypothesis is true, both methods control the type-I error rate at 0.025. In this case, ideally the sample size should not be increased. Without a futility rule, the design caps the new sample size at 800 in total (≈3 times of the planned 266) as a saving guard. It can be seen that the proposed continuous monitoring based on mTR method saves more by requesting much less increase (AS≈143-145%) than the conventional single-time snapshot analysis (AS≈183-189%), relative to the planned total of 266. If a futility rule (such as stop if new sample size exceeded 800) is incorporated, then more obvious advantage can be seen; futility monitoring is fully described in following examples.
(2) When the alternative hypothesis is true, both methods are able to request sample size increase since the planned sample size was based an over-estimate of the treatment effect. However, the proposed continuous monitoring based on mTR method requests much less sample size (≈58-59%) than the conventional single-time snapshot analysis (≈71-72%), relative to the ideal sample size of 672; each method targets its own conditional probability at 0.8. The shortage of reaching the 0.8 conditional probability is due to that cap of 800 patients.
(3) The continuous monitoring method conditioning on mTR≥0.2 sets a restrictive condition on when and whether to conduct SSR, as opposed to the conventional fixed schedule (t=½ or ¾) method which will conduct SSR without a restrictive condition. Under H₀ there is 50% chance the condition of mTR≥0.2 not being met during the trial thus no SSR being performed, as it should not be. (let τ=1 when no SSR is done). This is shown in Table 2, where τ=0.59 for the continuous monitoring method with the restrictive condition of mTR≥0.2 versus τ=0.5 for the fixed schedule t=½ method without a restrictive condition. Under H_{A}, however, it is more advantageous in trial operation and administration to perform a reliable SSR interim analysis earlier in time to determine whether and the amount of an increase of sample size is needed. Compared to the conventional single-time analysis at τ=0.5 or 0.75, the proposed continuous monitoring based on mTR method conducts the SSR much earlier at τ=0.34 (versus 0.5) or 0.32 (versus 0.75). The timing advantage of DAD/DDM in conducting SSR over the fixed schedule is very clearly demonstrated.

### Example 3

DAD/DDM with Consideration of Early Efficacy and Control of the Type-I Error Rate

The basis of DAD/DDM with continuous monitoring for early stop due to overwhelming evidence of efficacy is the seminal work of Lan, Rosenberger and Lachin (1993). DAD/DDM thus uses the continuous alpha-spending function α(t)=2{1−Φ(z_{1-α/2}/\sqrt{t})}, 0<t≤1, to ensure the control of the type-I error rate. Notice that α is the one-side level (usually 0.025) here. The corresponding Wald test Z-value boundary is the O'Brien-Fleming type boundary, which is often used in GSD and AGSD. For example, H₀ at α=0.025 would be rejected if

$$Z ( t ) \geq \frac{2.24}{\sqrt{t}} .$$

The second section of Example 1 discussed the formula for adjusting the critical value for the final test when SSR is performed after a GS boundary has been employed in the design for early efficacy monitoring and the final boundary value is C_{g}. For DAD/DDM with continuous monitoring, C_{g}=2.24.

On the other hand, if the continuous monitoring of efficacy is placed after SSR is performed (by either conventional CP_{\hat{θ}} or by CP_{mTR}) then the z_{1-α/2} quantile in the above alpha-spending function α(t) should be adjusted to C₁ as expressed in Eq. (3). Accordingly, the Z-value boundary would be adjusted to

$$\frac{{C}_{1}}{\sqrt{t}} .$$

The scale or the information fraction t would be based on the new maximum information I_{N}_{_{new}}.

TABLE 2
Total and conditional rate of rejecting H₀ (first and second columns)^{#}, AS = (average sample size)/672 for target conditional probability of 0.8
(third column), and timing (τ is information fraction conducting SSR) for SSR (fourth and fifth columns) averaged over 100,000 simulation runs.

| | | Total probability | Proportion of | Conditional Probability | | | |
|---|---|---|---|---|---|---|---|
| θ | SSR timing method | of rejecting H₀ | of mTR >=0.2 | of rejecting H₀ | AS (%) | τ * | τ ** |
| 0 | Single time point at t = 1/2⁺ | 0.025 | NA | NA | 486/266 = 183% | 0.50 | 0.50 |
| | mTR ≥0.2⁺⁺ | 0.025 | 0.50 | 0.044 | 380/266 = 143% | 0.59 | 0.18 |
| | Single time point at t = 3/4⁺ | 0.025 | NA | NA | 504/266 = 189% | 0.75 | 0.75 |
| | mTR ≥0.2⁺⁺⁺ | 0.025 | 0.51 | 0.045 | 386/266 = 145% | 0.59 | 0.19 |
| 0.25 | Single time point at t = 1/2⁺ | 0.775 | NA | NA | 478/672 = 71.1% | 0.5 | 0.5 |
| | mTR ≥0.2⁺⁺ | 0.651 | 0.81 | 0.741 | 390/672 = 58.0% | 0.34 | 0.18 |
| | Single time point at 3/4⁺ | 0.791 | NA | NA | 482/672 = 71.7% | 0.75 | 0.75 |
| | mTR ≥0.2⁺⁺⁺ | 0.660 | 0.85 | 0.744 | 398/672 = 59.2% | 0.32 | 0.20 |

1) Total Probability of rejection H₀: all rejection/simulations times (100000)
2) Condition rate: number of trials observing mTR ≥0.2/sim (100,000)
3) Conditional Probability of rejecting H₀: Rejection rates under situation of observing mTR ≥0.2
4) Average sample size (AS)/672: mean of all sample size (100,000) recorded/672
5) τ *: if trials don't observe mTR ≥0.2, then recorded as 1. Mean of information fraction from all simulations (100,000)
6) τ **: Mean of information fraction from only those with observing mTR ≥0.2.
${}^{\#} Rejecting {H}_{0} when \frac{{S}_{{N}_{new}}}{\sqrt{{I}_{{N}_{new}}}} \geq {C}_{1} , where 2 {N}_{new} is the new final total sample size capped at 800$ ⁺Eq. (1) with C₁ in Eq. (3) where C₀ = 1.96; t = iₙ_{_{E}}_{,n}_{_{C}}/I_{N}; using snap-shot point estimate of of \hat{θ} at t
⁺⁺mTR over TR(l) l = 10, 11, 12, . . . till t_{L} = t₃₃ using the average of the \hat{θ}'s, average Sₙ_{_{E}}_{,n}_{_{C}} and average iₙ_{_{E}}_{,n}_{_{C}} in the interval associated with mTR. τ = # of patients associated with mTR/266 or mTR/672
⁺⁺⁺mTR over TR(l) l = 10, 11, 12, . . . till t_{L} = t₅₀ using the average of the \hat{θ}'s, average Sₙ_{_{E}}_{,n}_{_{C}} and average iₙ_{_{E}}_{,n}_{_{C}} in the interval associated with mTR. τ = # of patients associated with mTR/266 or mTR/672

TABLE 3
Total Probability of rejection H₀: all rejection / simulations times (100000)

| | | Total probability of | Proportion of | Conditional Probability | | | |
|---|---|---|---|---|---|---|---|
| θ | SSR timing method | rejecting H₀ | minSR >= 1.02 | of rejecting H₀ | AS (%) | τ * | τ ** |
| 0 | Single time point at t = ½⁺ | 0.025 | NA | NA | 486/266 = 183% | 0.50 | 0.50 |
| | minSR ≥ 1.02⁺⁺⁺ | 0.025 | 0.57 | 0.028 | 526/266 = 197% | 0.59 | 0.28 |
| | Single time point at t = ¾⁺ | 0.025 | NA | NA | 504/266 = 189% | 0.75 | 0.75 |
| | minSR ≥ 1.02⁺⁺⁺ | 0.025 | 0.67 | 0.029 | 572/266 = 215% | 0.55 | 0.33 |
| 0.25 | Single time point at t = ½⁺ | 0.775 | NA | NA | 478/672 = 71.1% | 0.5 | 0.5 |
| | minSR ≥ 1.02⁺⁺⁺ | 0.801 | 0.66 | 0.864 | 534/672 = 79.5% | 0.53 | 0.28 |
| | Single time point at ¾⁺ | 0.791 | NA | NA | 482/672 = 71.7% | 0.75 | 0.75 |
| | minSR ≥ 1.02⁺⁺⁺ | 0.847 | 0.77 | 0.852 | 572/672 = 85.1% | 0.48 | 0.33 |

1) Condition rate: (# of trial with observing minSR ≥ 1.02) / sim (100,000)
2) Conditional Probability of rejecting H₀: Rejection rates under situation of observing minSR (minimum sample size ratio) ≥ 1.02
3) Average sample size /672: mean of all sample size (100,000) recorded /(266 or 672)
4) τ *: if trials don't observe minSR ≥ 1.02, then recorded as 1. Mean of information fraction from all simulations (100,000)
5) τ **: Mean of information fraction from only those with observing minSR ≥ 1.02.

In one embodiment, when using the continuous monitoring system of DAD/DDM, one may over-rule the suggestion of early stop when the efficacy boundary is crossed. Based on Lan, Lachine and Bautisa (2003), as one may over-rule an SSR signal recommended by the system. In this case, one may buy-back the previously spent alpha probability to be re-spent or re-distributed at future looks. Lan et al. (2003) showed that such plans using an O'Brien-Fleming-like spending function have a negligible effect on the final type I error probability and on the ultimate power of the study. They also showed that this approach can be simplified by using a fixed-sample size Z critical value for future looks after buying-back previously spent alpha (such as using a critical Z value of 1.96 for α=0.025.) This simplified procedure also preserves the type I error probability while incurring a minimal loss in power.

### Example 4

DAD/DDM with Consideration of Futility Decision

Several important aspects of futility interim analyses are worthy remarks. First, the SSR procedure discussed previously may also have implication on futility. If the re-estimated new sample size exceeds multiple folds of the originally planned sample size, beyond the feasibility of conducting the trial, then the sponsor may likely deem the trial futile. Second, futility analyses are sometimes imbedded in efficacy interim analyses. However, since the decision of whether a trial is futile (thus stop the trial) or not (thus continue the trial) is non-binding, futility analysis plan should not be used to buy back the type-I error rate. Rather, futility interim analyses increase the type-II error rate, thus induce power loss of the study. Third, when futility interim analysis is separately conducted from the SSR and efficacy analyses, the optimal strategy of futility analyses, including timing and criterion, should be considered to minimize cost and power loss. By analyzing the accumulative data continuously after each patient entry, it is conceivable that DAD/DDM can monitor futility more reliably and rapidly than the occasional, snap-shot interim analysis can. This section first reviews the optimal timing of futility analyses for occasional data monitoring, and then discusses the DAD/DDM procedure with continuous monitoring. The two methods, occasional and continuous monitoring, are compared by simulation studies.

### Optimal Timing of Futility Interim Analysis for Occasional Data Monitoring

In conducting SSR, the present invention secures study power by properly increasing the sample size, while guard against unnecessary increase if the null hypothesis is true. Conventional SSR is usually conducted at some mid time-point such as t=½, but no later than t=¾. In futility analysis, the procedure can spot the hopeless situation as early as possible to save cost as well human suffering from ineffective therapy. One the other hand, futility analysis induces power loss; frequent futility analyses induce excessive power loss. Thus, the present invention can frame the timing issue of futility analyses as an optimization problem by seeking minimization of the sample size (cost) as the objective while controlling the power loss. This approach has been taken by Xi, Gallo and Ohlssen (2017).

Futility Analysis with Acceptance Boundaries in GS Trials

Suppose that sponsor wants to schedule K−1 futility interim analyses in a GS trial at information fraction time tₖ with total cumulative information iₖ from sample size nₖ, k=1, . . . , K −1, respectively. Let the futility boundary value be bₖ at information fraction time

$${t}_{k} = \frac{{i}_{k}}{{I}_{K}} ,$$

k=1, . . . , K −1. (i_{K}=Iₖ and t_{K}=1). Thus the study is stopped at time tₖ if Zₖ≤bₖ and conclude futility for the test treatment; otherwise the clinical trial continues to the next analysis. At the final analysis, H₀ would be rejected if Z_{K}>z_{α} and otherwise accept H₀. Notice that the final boundary value is still z_{α} as remarked in the beginning of this section.

The expected total information is given by ETI_{θ}=Σₖ₌₁^{K-1}P (stop at tₖ for the first time|θ)+I_{K}P(never stop at any interim analysis|θ)=I_{K}Σₖ₌₁^{K-1}tₖP(Zₖ≤bₖ at tₖ for the first time|θ)+I_{K}P(never stop at any interim analysis|θ)

The expected total information may also be expressed as a percentage of the maximum information as ETI_{θ}(%)=ETI_{θ}/I_{K}.

The power of this GS trial is P[(Z_{K}>z_{α})∩ₖ₌₁^{K-1}(Zₖ>bₖ)|θ=θ*]

Compared to power of the fixed sample size design without interim futility analyses, which is U=P(Z>z_{α}|θ=θ*), the power loss due to stopping for futility is given by PL=U −P[Z_{K}>z_{α})∩ₖ₌₁^{K-1} (Zₖ>bₖ)|θ=θ*)

It can be seen that the higher dₖ, the easier to reach futility and stop, the more power loss. For a given boundary value bₖ, since Zₖ˜N(θ\sqrt{Iₖ},1), the smaller Iₖ (the earlier futility analysis), also the easier to reach futility and stop, the larger the power loss. However, if the null hypothesis is true, the earlier interim analysis, the smaller ETI₀, the more saving on the cost.

Therefore, (tₖ, bₖ) k=1, . . . , K −1, is searched to minimize ETI₀ such that PL≤λ. Here λ is a design choice for protection of power loss from the futility analysis that may incorrectly terminate a positive trial. Xi, Gallo and Ohlssen (2017) investigated optimal timing subject to various tolerable power loss λ and using the Gamma (γ) family of Hwang, Shih and DeCani (1990) as the boundary values.

For a single futility analysis, in particular, the task can be accomplished without restricting to a functional form of futility boundary. That is, (t₁, b₁) can be found to minimize ETI₀=[t₁Φ(b₁)+1−Φ(b₁)] such that that PL=P(Z₁≤d₁, Z₂>z_{α}|θ=θ*)≤λ. For a given λ and z_{α} to detect θ*, a grid search can be done among 0.10≤t₁≤0.80 (using an increment of 0.05 or 0.10) for the corresponding boundary value b₁.

For example, for a design with z_{α}=1.96 to detect θ*=0.25, if a λ=5% power loss is allowed, then the optimal timing is achieved by setting the futility boundary b₁=0.70 at t₁=0.40 (using an increment of 0.10 in grid search). The cost saving measured by the expected total information under the null hypothesis, expressed as a percentage of the fixed sample size design, is ETI₀=54.5%. If only λ=1% power loss is allowed, then the optimal timing is achieved by b₁=0.41 at t₁=0.50 with the same grid search. The cost saving is ETI₀=67.0%.

Next the robustness of the above optimization shall be considered on timing the futility analysis and associated boundary value. Suppose the optimal timing is designed with associated boundary value, but in practice when monitoring the trial, the timing of futility analysis may not on the designed schedule. What does the present invention do? Usually the original boundary value is desired to be kept (since it is often already documented in the statistical analysis plan), then the change in the power loss and ETI₀ can shall be investigated. Xi, Gallo and Ohlssen (2017) reported the following: In design, a λ=1% power loss is specified, leading to an optimal timing at t₁=0.50 with b₁=0.41. The cost saving is ETI₀=67.0%. (See previous paragraph). Suppose that during monitoring the actual time of the futility analysis is some t between [0.45, 0.55]. The z-scale boundary b₁=0.41 is kept as in the plan. As the actual time t deviates from 0.50 toward earlier time 0.45, the power loss increases slightly from 1% to 1.6%, and ETI₀ decreases slightly from 67% to 64%. As the actual time t deviates from 0.50 toward later time 0.55, the power loss decreases slightly from 1% to 0.6% and ETI₀ increases slightly from 67% to 70%. Therefore, the optimal futility rule (t₁=0.50, b₁=0.41) is very robust.

Furthermore, robustness of the optimal futility rule shall also be examined regarding the treatment effect assumption of θ* in the design. Xi, Gallo and Ohlssen (2017) considered optimal futility rules that yield power loss ranging from 0.1% to 5% with assumed θ*=0.25. For each level of these power loss, compare it with that calculated with θ=0.2, 0.225, 0.275, and 0.25, respectively. It was shown that the magnitude of power loss was quite close to each other. For example, for the maximum power loss of 5% with assumed θ*=0.25, the actual power loss is 5.03% if the actual θ=0.2, and the actual power loss is 5.02 if the actual θ=0.275.

Futility Analysis with Conditional Power Approach

Another approach for GS trial with futility consideration is to use the conditional power

$$P ( \frac{{S}_{N}}{\sqrt{{I}_{N}}} \geq C \vert {S}_{{n}_{E} , {n}_{C}} = u )$$

seen in Eq. (1) for N=N₀. If the conditional power under Hₐ is lower than a threshold (γ), then the trial is deemed hopeless and may be stopped for futility. Fixing γ, u is the futility boundary for Sₙ_{_{E}}_{,n}_{_{C}}. If the original power is 1−β, applying result given in Lan, Simon and Halperin (1982), the power loss would be at most

$$β ( \frac{1}{γ} - 1 ) .$$

For example, for a trial with original power of 90%, designing an interim futility analysis using conditional power approach with futility cutoff γ=0.40, the power loss is at most 0.14.

Similarly, if the SSR based on

$$P ( \frac{{S}_{N}}{\sqrt{{I}_{N}}} \geq C \vert {S}_{{n}_{E} , {n}_{C}} = u )$$

for N=N_{new} gives a new sample size that exceeds multiple folds of the original sample size to provide a target power, then the trial is also deemed hopeless and may be stopped for futility.

### Optimal Timing of Futility Interim Analysis for Continuous Monitoring

For continuous monitoring with conditional power expressed in Eq. (1), the “trend ratio based conditional power”

$$C {P}_{T R ( N )} = P ( \frac{S ( {I}_{N} )}{\sqrt{{I}_{N}}} \geq C \vert a \leq max \{ T R ( l ) , l = 10 , 11 , 12 , \text{...} \} < b )$$

where N=N₀ or N_{new} is used. As before, instead of using a single point estimate of

$$\hat{θ} = \frac{{S}_{{n}_{E} , {n}_{C}}}{{i}_{{n}_{E} , {n}_{C}}} ,$$

Sₙ_{_{E}}_{,n}_{_{C}} and iₙ_{_{E}}_{,n}_{_{C}}, the average of the \hat{θ}'s as well as the average Sₙ_{_{E}}_{,n}_{_{C}} and iₙ_{_{E}}_{,n}_{_{C}} are used, respectively, in the interval associated with the mTR. If CP_{TR(N}_{_{new}}₎ is lower than a threshold, then the trial is deemed hopeless and may be stopped for futility. If CP TR(N_{new}) to provide a target power requires N_{new} that exceeds multiple folds of N₀, then the trial is also deemed hopeless and may be stopped for futility. This is SSR with futility as opposed to the “pure” SSR discussed in Section 4. The timing of SSR discussed in Section 4 thus also is the time to perform futility analysis. That is, the futility analysis is conducted at the same time when SSR is being conducted. Since futility analysis and SSR are non-binding, the present invention can monitor the trial as it proceeds without affecting the type-I error. However, futility analysis decreases the study power, and sample size should be increased at most once during the trial for feasible operation. These should be considered with caution.

### Comparison of Futility Analysis Using Trend to GS

Following the same setup as in Example 2, the conventional SSR is usually conducted at some mid time-point when t≈½. DAD/MMD uses trend analysis over several time-points as described previously. Both use conditional power approach, but utilize different amount of data in estimating the treatment effect. The two methods are compared by simulation as follows. Assume a clinical trial with true θ=0.25 and common variance=1. (The same set up as in Sections 3.2 and 4). Here, a sample size of N=336 per arm (672 total) is ideally needed with 90% power at α=0.025 (one-sided). However, it is assumed that θ_{assumed}=0.4 in planning the study and the planned sample size of N=133 per arm (266 total) is used with randomization of block size 4. These two situations are compared: monitoring the trial continuously after each patient entry with the DAD/MDD procedure versus the conventional SSR procedure with futility considerations. Specifically, with the conventional SSR procedure, SSR+futility analysis is conducted at either t≈½ (N=66 per arm or 132 in total) using the snap-shot point estimate \hat{θ} at t ≈1/2. If conditional power under θ_{assumed}=0.4 is less than 40% or the total new sample size exceeds 800, then the trial is stopped for futility. In addition, if \hat{θ} is negative when conducting SSR, the trial is deemed futile too. In one embodiment, the present invention uses the bench mark result from Xi, Gallo and Ohlssen (2017) that the smallest average sample size (67% of the total 266) with 1% power loss is achieved by a futility boundary z=0.41 at 50% information.

With the DAD/DDM, there is no pre-specified time-point to conduct SSR but the timing with mTR is monitored, in which calculation of TR(l) starts at tₗ=t₁₀ with every 4 patients entry (hence total=40 patients at t₁₀). For timing by mTR, the calculation moves along t₁₀, t₁₁, . . . t_{L} and find max of TR(l) over 1, 2, . . . L−9 segments, respectively, until the first time mTR≥0.2 or till t≈½ (132 patients in total) where t_{L}=t₃₃ and the max would be over 33−9=24 segments—to compare with the above conventional t≈½ method. Only at the first mTR≥0.2 will the new sample size be calculated with Eq. (2) using the average of the \hat{θ}'s as well as the average Sₙ_{_{E}}_{,n}_{_{C}} and average iₙ_{_{E}}_{,n}_{_{C}} in the interval associated with mTR. If CP_{TR(N}_{₀}₎ is lower than 40%, or CP_{TR(N}_{_{new}}₎ to provide a target power of 80% requires N_{new} that exceeds 800 total, then the trial is stopped for futility. If till t=0.90 still mTR<0.2 then stop the trial for futility. In addition, if the average \hat{θ} is negative, the trial is deemed futile too.

The power loss, average sample size, and timing for these procedures are compared under θ=0, 0.25, and 0.40

Under the null hypothesis, the score function S(t)˜N(0, t). This means that the trend of the trajectory of S(t) is horizontal and the curve should be below zero half of the times. If the intervals are denoted on which S(t)≤0 as I_{0,1}, I_{0,2}, . . . , with lengths |I_{0,1}|, |I_{0,2}|, . . . , then E(Σᵢ|I_{0,i}|/t)=0.5. Therefore, if Σᵢ|I_{0,i}|/t is observed to be close to 0.5, then the trial will more than likely be futile. Furthermore, the Wald statistics Z(t)=S(t)/\sqrt{t}˜N(0,1) also shares the same characteristic. So, the same ratio from the Wald statistic can be used for futility evaluation. Similarly, number of observations that crossed below zero by either S(t) or Z(t) can be used for futility determination.

Table 4 shows indeed that the number of observed negative values has high specificity of separating the null (θ=0) from the alternative (θ>0). For example, using 80 times of S(t) or Z(t) below zero by time t as the cut-off for futility, the chance of correct decision is 77.7% versus wrong decision is 8% if θ=0.2. It is shown by more simulation that DAD/DDM performs better than the occasional, snap-shot monitoring for futility.

TABLE 4
Probability of futility stop using number of times S(t)
below zero (100,000 simulations)

| Futility stop by # of | θ = 0 | θ = 0.2 | θ = 0.3 | θ = 0.4 | θ = 0.5 | θ = 0.6 |
|---|---|---|---|---|---|---|
| times S(t) below zero | (%) | (%) | (%) | (%) | (%) | (%) |
| 10 | 91.7 | 43.6 | 27.51 | 17.13 | 9.32 | 5.4 |
| 20 | 87.0 | 30.6 | 10.6 | 5.7 | 3.6 | 1.5 |
| 30 | 82.7 | 24.4 | 7.5 | 4.1 | 1.0 | 0.5 |
| 40 | 82.0 | 19.2 | 5.6 | 1.2 | 0.9 | 0.0 |
| 50 | 80.2 | 15.0 | 3.5 | 0.5 | 0.0 | 0.0 |
| 60 | 79.0 | 11.9 | 3.0 | 0.3 | 0.0 | 0.0 |
| 70 | 76.9 | 10.1 | 1.4 | 0.2 | 0.0 | 0.0 |
| 80 | 77.7 | 8.0 | 1.5 | 0.3 | 0.0 | 0.0 |

Since the scores are calculated whenever new random samples are drawn, the futility ratio can be calculated at time t, FR(t), as follows: FR(t)=(# of S(t)=<0)/(# of S(t) calculated).

### Example 5

Making Inference when Using DAD/DDM with SSR

The DAD/DDM procedure assumes that there is an initial sample size N=N₀, with corresponding Fisher's information T₀, and that the score function S(t)≈B(t)+θt˜N(θt,t) is continuously calculated as data accumulate with the trial enrollment. Without any interim analysis, if the trial ends at the planned information time T₀, and S(T₀)=u_{T}_{₀}, then the null hypothesis is rejected if

$$\frac{S ( {T}_{0} )}{\sqrt{{T}_{0}}} \geq {Z}_{1 - α} = {C}_{0} .$$

For inferences (point estimate and confidence intervals), it is defined as

$$f ( θ ) = {P ( \frac{S ( {T}_{0} )}{\sqrt{{T}_{0}}} \geq \frac{{u}_{{T}_{0}}}{\sqrt{{T}_{0}}} )}_{θ} = P ( B ( {T}_{0} ) + θ {T}_{0} \geq {u}_{{T}_{0}} ) = 1 - φ ( \frac{{u}_{{T}_{0}} - θ {T}_{0}}{\sqrt{{T}_{0}}} ) .$$

Then f(θ) is an increasing function of θ, and f(0) is the p-value. Let θ_{γ}=f⁻¹(γ). Then

$${θ}_{0.5} = \frac{S ( {T}_{0} )}{{T}_{0}} ∼ N ( θ , \frac{1}{{T}_{0}} ) ,$$

and the Maximum Likelihood Estimator (MLE) is a median unbiased estimate of θ. The confidence limits are

$${θ}_{α} = {θ}_{0.5} - {Z}_{1 - α} \frac{1}{\sqrt{{T}_{0}}} and {Z}_{1 - α} = {θ}_{0.5} + {Z}_{1 - α} \frac{1}{\sqrt{{T}_{0}}} .$$

The two-sided confidence interval has exact (1 −2α)×100% coverage.

The adaptive procedure allows the sample size to be changed at any time, say at t₀ with observed score S(t₀)=uₜ_{₀}. Suppose the new information is T₁, which corresponds to sample size N₁. Let S(T₁) be the potential observation at T₁. To preserve the type-I error rate, the final critical boundary Z_{1-α}=C₀ must be adjusted to C₁, which satisfies P(S(T₁) >C₁≥C₁\sqrt{T₁}|S(t₀)=uₜ_{₀})=P(S(T₀)≥C₀\sqrt{T₀}|S(t₀)=uₜ_{₀}), using the independent increment property of Brownian motions, which can be solved as

$${C}_{1} = \frac{1}{\sqrt{{T}_{1}}} \{ \frac{\sqrt{{T}_{1} - {t}_{0}}}{\sqrt{{T}_{0} - {t}_{0}}} ( {C}_{0} \sqrt{{T}_{0}} - {u}_{{t}_{0}} ) \} + \frac{{u}_{{t}_{0}}}{\sqrt{{T}_{1}}} \qquad ( 2 )$$

Note that Chen, DeMets and Lan (2004) showed that if the conditional power using the current point estimate of θ at t₀ is at least 50%, then increasing sample size will not inflate the type-I error, hence there is no need to change the C₀ to C₁ for the final test.

Let the final observation be S(T₁)=u_{T}_{₁}. The null hypothesis will be rejected if

$$\frac{S ( {T}_{1} )}{\sqrt{{T}_{1}}} \geq {C}_{1} .$$

For any hypothesized value θ, a “backward image” is identified (denoted as u_{T}_{₀}^{BK}; see Gao, Liu, Mehta, 2013). u_{T}_{₀}^{BK} satisfies the relationship P(S(T₁)≥u_{T}_{₁}|S(t₀)=uₜ_{₀})=P(S(T₀)≥u_{T}_{₀}^{BK}|S(t₀)=uₜ_{₀}), which can be solved as

$${u}_{{T}_{0}}^{B K} = \{ \frac{\sqrt{{T}_{1} - {t}_{0}}}{\sqrt{{T}_{0} - {t}_{0}}} ( {u}_{{T}_{1}} - {u}_{{t}_{0}} + θ ( {T}_{1} - {t}_{0} ) ) \} + {u}_{{t}_{0}} + θ ( {T}_{0} - {t}_{0} ) .$$

TABLE 5
Point estimate and CI coverage (up to two sample size modifications)

| True θ | Median (\hat{θ}) | CI coverage | θ < θ_{α} | θ > θ_{1-α} |
|---|---|---|---|---|
| 0.0 | 0.0007 | 0.9494 | 0.0250 | 0.0256 |
| 0.2 | 0.1998 | 0.9471 | 0.0273 | 0.0256 |
| 0.3 | 0.2984 | 0.9484 | 0.0253 | 0.0264 |
| 0.4 | 0.3981 | 0.9464 | 0.0278 | 0.0259 |
| 0.5 | 0.5007 | 0.9420 | 0.0300 | 0.0279 |
| 0.6 | 0.5984 | 0.9390 | 0.0307 | 0.0303 |

Let

$$f ( θ ) = P ( B ( {T}_{0} ) + θ {T}_{0} \geq {u}_{{T}_{0}}^{B K} ) = 1 - Φ ( \frac{{u}_{{T}_{0}}^{B K}}{\sqrt{{T}_{0}}} - θ \sqrt{{T}_{0}} ) .$$

Then f(θ) is an increasing function, and f(0) is the p-value. Let θ_{γ}=f⁻¹(γ).

$${θ}_{0.5} = \frac{{u}_{{T}_{0}}^{B K}}{{T}_{0}}$$

is a median unbiased estimate of θ·(θₐ, θ₁₋ₐ) is an exact two−sided 100%×(1−2α) confidence interval.

Table 5 presents simulations that confirm that the point estimate is median unbiased and the two-sided confidence interval has exact coverage. The random samples are taken from normal distributions N(θ, 1), and the simulations are repeated 100,000 times.

### Example 6

### Comparison of AGSD and DAD/DDM

The present invention first describes the performance metric for a meaningful comparison between AGSD and DAD/DDM, followed by description of the simulation study, then the results.

### Metric for Design Performance

An ideal design would be able to provide adequate power (P) without requiring excessive sample size (N) for a range of effect sizes (θ) that are clinically beneficial. To be more specifically, the concept is illustrated in FIG. 3 with the following explanations:
It is common to design a trial with target power, say, at P₀=0.9 with some leeway such that P₀−Δ≤P (say Δ=0.1) is acceptable, but P<P₀−Δ (area A₁) will not be acceptable. For example, desired power is 0.9, but 0.8 is still acceptable.
Let Nₚ be the sample size that provides power P with a fixed sample design. Designs with P₀>0.9 are rarely seen since Nₚ will need to be much larger than N_{0.9} (i.e., it requires a large sample size increase over N_{0.9} to gain small additional power beyond 0.9. Such sample sizes can be infeasible in rare diseases or trials in which the per-patient cost is high). A sample size N larger than (1+r₁)N_{0.9} (say, r₁=0.5) may be considered excessively large, hence unacceptable (area A₂), even if the power provided by this sample size is slightly more than 0.9. For example, a design that requires a sample size of N_{0.999} to provide P=0.999 power would not be a desirable design. On the other hand, a sample size N<(1+r₁)N_{0.9} can be considered to be acceptable if it provided at least 0.9 power.
Another unacceptable situation is that, although the power is acceptable (but not ideal) at 0.8<P<0.9, the sample size is not “economical”. Such an example is that when N >(1+r₂)N_{0.9} (say, r₂=0.2). The unacceptable area is A₃ as shown.

These criteria for acceptance are applied to a range of effect sizes θ∈(θ_{low},θ_{high}), where θ_{low} is the smallest effect size that is clinically relevant.

The cutoffs such as P₀, Δ, or r₁, r₂ depend on many factors including the cost and feasibility, unmet medical need, etc. The above discussion suggests that the performance of a design (either fixed sample design, or a non-fixed sample design) involves three parameters, namely (θ, P_{d}, N_{d}), where θ∈(θ_{low},θ_{high}), P_{d} is the power provided by the design “d”, and N_{d} is the required sample size associated with P_{d}. Hence the evaluation of the performance of a given design is a three-dimensional issue. The Performance Score of design is defined as following and also illustrated in a figure below.

$$PS ( θ ) = \{ \begin{matrix}- 1 , & ( {P}_{d} , {N}_{d} ) ∈ ( {A}_{1} ⋃ {A}_{2} ⋃ {A}_{3} ) \\ 0 , & ( {P}_{d} , {N}_{d} ) ∈ ( {B}_{1} ⋃ {B}_{2} ⋃ {B}_{3} ) \\ 1 , & ( {P}_{d} , {N}_{d} ) ∈ C\end{matrix}$$

Previously, Liu et al (2008) and Fang et al (2018) both used one-dimensional scales to evaluate the performance of different designs. Both scales are difficult to interpret since they reduced three-dimensional aspects of performance to a one-dimensional metric. The performance score preserves the three-dimensional nature of design performance and it is easy for interpretation.

Simulation studies are conducted to compare AGSD and DAD/DDM as follows. In the simulations, θ_{assumed}=0.4, and the initial planned sample size was N=133 per arm to provide a 90% power (1-sided alpha=0.025) if the treatment effect is correctly assumed. Random samples were drawn from N(θ, 1), with (true) θ=0, 0.2, 0.3, 0.4, 0.5, 0.6. Sample size was capped at N=600 per arm. The performance score was calculated for each scenario with 100,000 simulation runs, there is no alpha buy-back with futility stopping, as futility stopping is usually considered non-binding.

### Simulation Rules for AGSD

Simulations require automated rules, which are usually simplified and mechanical. In the simulations for AGSD, rules commonly used in practice are used. These rules are: (i) Two looks, interim analysis at 0.75 of information fraction. (ii) SSR performed at the interim analysis (e.g., Cui, Hung, Wang, 1999; Gao, Ware, Mehta, 2008). (iii) Futility stop criterion: \hat{θ}<0 at the interim analysis.

### Simulation Rules for DAD/DDM

In our simulations for DAD/DDM, a set of simplified rules was used to make automated decisions. These rules are (in parallel and contrast to the AGSD): (i) Continuous monitoring through information time t, 0<t≤1. (ii) Timing the SSR by using the values of r. SSR, when performed, to achieve conditional power of 90%. (iii) Futility stop criterion: at any information time t, 80 times or more that \hat{θ}<0 during the time interval (0, t).

### Simulation Results

TABLE 6
Comparison of ASD and DDM

| Fixed | | | | | | | | | |
|---|---|---|---|---|---|---|---|---|---|
| sample | | ASD | | | | DDM | | | |
| Actual θ | SS | AS-SS | SP | FS | PS | AS-SS | SP | FS | PS |
| 0.00 | NA | 325 | 0.0257 | 49.8 | NA | 280 | 0.0248 | 74.8 | NA |
| 0.20 | 526 | 363 | 0.7246 | 8.20 | −1 | 399 | 0.8181 | 7.10 | 0 |
| 0.30 | 234 | 264 | 0.9547 | 1.76 | 0 | 256 | 0.9300 | 1.80 | 0 |
| 0.40 | 133 | 171 | 0.9922 | 0.25 | 0 | 157 | 0.9230 | 0.40 | 0 |
| 0.50 | 86 | 119 | 0.9987 | 0.03 | 0 | 106 | 0.9140 | 0.00 | 0 |
| 0.60 | 60 | 105 | 0.9999 | 0.00 | −1 | 79 | 0.9130 | 0.00 | 0 |

Note:
AS-SS = Avg. simulated SS; SP = simulated power; FS = Futility stop (%).

Table 6 shows simulation study of 100,000 runs to compare the ASD and DDM in term of futility stopping rate under H₀, average sample size, simulated power gained and the design performance. It clearly shows that DDM has higher futility stopping rate (74.8%), needs fewer sample size to gain desirable power and with acceptable performance.
For the null case (θ=0), the type I error is properly controlled by both AGSD and DAD/DDM. The trend-based futility stopping rule of DAD/DDM is more specific and reliable than the single-point snap-shot analysis used by AGSD. As a result, the futility stopping rate is much higher for DAD/DDM than for AGSD, and the sample size under the null for the DAD/DDM is smaller than that for AGSD.
For θ=0.2, AGSD does not provide acceptable power. For θ=0.6, AGSD results in excessive sample size. In both of these extreme cases, the performance scores of AGSD are rated as PS=−1, while for DAD/DDM they are acceptable (PS=0). For the other in-between cases θ=0.3, 0.4, and 0.5, AGSD and DAD/DDM both performed acceptably in terms of achieving the target conditional power with reasonable sample size adjustment.

In summary, the simulations show that if the effect size is incorrectly assumed in a trial design:
i) The DAD/DDM can guide the trial to a proper sample size to provide adequate power for all possible true effect size scenarios.
ii) AGSD adjusts poorly if the true effect size is either much smaller or much larger than the assumed. In the former case, AGSD provides less than the acceptable power, while in the latter case, it requests excessive sample size.

### Proof of Probability Calculation Using Backward Image

### A Median Unbiased Point Estimate

Suppose that there is one sample size change for W(⋅), given an observation Sₜ_{₀}=uₜ_{₀}, the sample size (information time) is changed to T₁, and S_{T}_{₁}=U_{T}_{₁} is observed. Then a backward image u_{T}_{₀}^{BK} is obtained. Note that W(T₀) ˜N(θT₀,T₀) and

$$\frac{W ( {T}_{0} ) - θ {T}_{0}}{\sqrt{{T}_{0}}} \text{∼} N ( 0 , 1 )$$

$${f}_{{u}_{{T}_{1}}} ( θ ) = f ( θ , {u}_{{T}_{1}} ) = P ( W ( {T}_{0} ) \geq {u}_{{T}_{0}}^{B K} ) = 1 - Φ ( \frac{{u}_{{T}_{0}}^{B K}}{\sqrt{{T}_{0}}} - θ \sqrt{{T}_{0}} )$$

For any given u_{T}_{₀}^{BK}, f(θ,u_{T}_{₁})=f(θ,u_{T}_{₀}^{BK}) is an increasing function of θ and a decreasing function of u_{T}_{₀}^{BK}. For any 0<γ<1, let

$${θ}_{γ} ( {u}_{{T}_{1}} ) = {f}_{{u}_{{T}_{1}}}^{- 1} ( γ ) .$$

Then f⁻¹(θ_{γ},u_{T}_{₁})=f⁻¹(θ_{γ},u_{T}_{₀}^{BK})=γ. Thus

$$γ = 1 - Φ ( \frac{{u}_{{T}_{0}}^{B K}}{\sqrt{{T}_{0}}} - {θ}_{γ} \sqrt{{T}_{0}} ) and {θ}_{γ} = \frac{{u}_{{T}_{0}}^{B K} - {Φ}^{- 1} ( 1 - γ )}{{T}_{0}} .$$

Note that θ_{γ}(u_{T}_{₁})=θ_{γ}(u_{T}_{₀}^{BK}). Let u_{γ}=θT₀+\sqrt{T₀}Φ⁻¹ (1−γ). Then

$$f ( θ , {u}_{γ} ) = 1 - Φ ( \frac{{u}_{γ}}{\sqrt{{T}_{0}}} - {θ}_{γ} \sqrt{{T}_{0}} ) = γ . \\ P ( {θ}_{γ} \leq θ ) = P ( f ( {θ}_{γ} , {u}_{{T}_{0}}^{B K} ) \leq f ( θ , {u}_{{T}_{0}}^{B K} ) ) = P ( γ \leq f ( θ , {u}_{{T}_{0}}^{B K} ) ) \\ = P ( f ( θ , {u}_{γ} ) \leq f ( θ , {u}_{{T}_{0}}^{B K} ) ) = P ( {u}_{γ} \geq {u}_{{T}_{0}}^{B K} ) = P ( {u}_{γ} \geq W ( {T}_{0} ) ) \\ = Φ ( \frac{{u}_{γ}}{\sqrt{{T}_{0}}} - θ \sqrt{{T}_{0}} ) = 1 - γ$$

### And

Hence,

$$( {θ}_{0.5} \leq θ ) = 0.5 , P ( {θ}_{1 - \frac{α}{2}} \leq θ ) = \frac{α}{2} , P ( θ \leq {θ}_{\frac{α}{2}} ) = 1 - P ( {θ}_{\frac{α}{2}} \leq θ ) = \frac{α}{2} .$$

Thus θ_{0.5} is a median unbiased estimate of θ, and

$$( {θ}_{\frac{α}{2}} , {θ}_{1 - \frac{α}{2}} )$$

is an exact two-sided 100%×(1−α) confidence interval.

### Backward Image Calculation

Estimates with One Sample Size Modification

Let

$$f ( {θ}_{γ} ) = 1 - Φ ( \frac{{u}_{N}^{B K}}{\sqrt{{T}_{N}}} - {θ}_{γ} \sqrt{{T}_{N}} ) = γ$$

Solve for θ_{γ}:

$${θ}_{γ} = \frac{\frac{\sqrt{{T}_{N} - {t}_{{n}_{E} , {n}_{C}}}}{\sqrt{{T}_{{N}_{n e w}} - {t}_{{n}_{E} , {n}_{C}}}} ( {u}_{{N}_{n e w}} - {u}_{{n}_{E} , {n}_{C}} ) + {u}_{{n}_{E} , {n}_{C}} + {Z}_{1 - γ} \sqrt{{T}_{N}}}{\sqrt{{T}_{N} - {t}_{{n}_{E} , {n}_{C}}} \sqrt{{T}_{{N}_{n e w}} - {t}_{{n}_{E} , {n}_{C}}} + {t}_{{n}_{E} , {n}_{C}}}$$

Hence,

$${θ}_{0.5} = \frac{\frac{\sqrt{{T}_{N} - {t}_{{n}_{E} , {n}_{C}}}}{\sqrt{{T}_{{N}_{n e w}} - {t}_{{n}_{E} , {n}_{C}}}} ( {u}_{{N}_{n e w}} - {u}_{{n}_{E} , {n}_{C}} ) + {u}_{{n}_{E} , {n}_{C}}}{\sqrt{{T}_{N} - {t}_{{n}_{E} , {n}_{C}}} \sqrt{{T}_{{N}_{n e w}} - {t}_{{n}_{E} , {n}_{C}}} + {t}_{{n}_{E} , {n}_{C}}}$$

$$and$$

$${θ}_{\frac{α}{2}} = {θ}_{0.5} - \frac{{Z}_{1 - \frac{α}{2}} \sqrt{{T}_{N}}}{\sqrt{{T}_{N} - {t}_{{n}_{E} , {n}_{C}}} \sqrt{{T}_{{N}_{n e w}} - {t}_{{n}_{E} , {n}_{C}}} + {t}_{{n}_{E} , {n}_{C}}}$$

$${θ}_{1 - \frac{α}{2}} = {θ}_{0.5} + \frac{{Z}_{1 - \frac{α}{2}} \sqrt{{T}_{N}}}{\sqrt{{T}_{N} - {t}_{{n}_{E} , {n}_{C}}} \sqrt{{T}_{{N}_{n e w}} - {t}_{{n}_{E} , {n}_{C}}} + {t}_{{n}_{E} , {n}_{C}}}$$

Estimates with Two Sample Size Modification

For the final inference, let

$$f ( {θ}_{γ} ) = 1 - Φ ( \frac{{u}_{N}^{B K}}{\sqrt{{T}_{N}}} - {θ}_{γ} \sqrt{{T}_{N}} ) = γ .$$

θ_{γ} can be solved as

$${θ}_{γ} = \frac{( \frac{\sqrt{{T}_{N} - {t}_{{n}_{E , 1} , {n}_{C , 1}}}}{\sqrt{{T}_{{N}_{new , 1}} - {t}_{{n}_{E , 1} , {n}_{C , 1}}}} ( \frac{\sqrt{{T}_{{N}_{new , 1}} - {t}_{{n}_{E , 2} , {n}_{C , 2}}}}{\sqrt{{T}_{{N}_{new , 2}} - {t}_{{n}_{E , 2} , {n}_{C , 2}}}} ( {u}_{{N}_{new , 2}} - {u}_{{{n}_{E , 2}}^{, n} C , 2} ) + {u}_{{n}_{E , 2} , {n}_{C , 2}} - {u}_{{n}_{E , 1} , {n}_{C , 1}} ) + {u}_{{n}_{E , 1} , {n}_{C , 1}} ) - {Z}_{1 - γ} \sqrt{{T}_{N}}}{\frac{\sqrt{{T}_{N} - {t}_{{n}_{E , 1} , {n}_{C , 1}}}}{\sqrt{{T}_{{N}_{new , 1}} - {t}_{{n}_{E , 1} , {n}_{C , 1}}}} ( \sqrt{{T}_{{N}_{new , 1}} - {t}_{{n}_{E , 2} , {n}_{C , 2}}} \sqrt{{T}_{{N}_{new , 2}} - {t}_{{n}_{E , 2} , {n}_{C , 2}}} + {t}_{{n}_{E , 2} , {n}_{C , 2}} - {t}_{{n}_{E , 1} , {n}_{C , 1}} ) + {t}_{{n}_{{E}^{, n} C}}}$$

Hence,

$${θ}_{0.5} = \frac{( \frac{\sqrt{{T}_{N} - {t}_{{n}_{E , 1} , {n}_{C , 1}}}}{\sqrt{{T}_{{N}_{new , 1}} - {t}_{{n}_{E , 1} , {n}_{C , 1}}}} ( \frac{\sqrt{{T}_{{N}_{new , 1}} - {t}_{{n}_{E , 2} , {n}_{C , 2}}}}{\sqrt{{T}_{{N}_{new , 2}} - {t}_{{n}_{E , 2} , {n}_{C , 2}}}} ( {u}_{{N}_{new , 2}} - {u}_{{{n}_{E , 2}}^{, n} C , 2} ) + {u}_{{{n}_{E , 2}}^{, n} C , 2} - {u}_{{{n}_{E , 1}}^{, n} C , 1} ) + {u}_{{{n}_{E , 1}}^{, n} C , 1} )}{\frac{\sqrt{{T}_{N} - {t}_{{n}_{E , 1} , {n}_{C , 1}}}}{\sqrt{{T}_{{N}_{new , 1}} - {t}_{{n}_{E , 1} , {n}_{C , 1}}}} ( \sqrt{{T}_{{N}_{new , 1}} - {t}_{{n}_{E , 2} , {n}_{C , 2}}} \sqrt{{T}_{{N}_{new , 2}} - {t}_{{n}_{E , 2} , {n}_{C , 2}}} + {t}_{{n}_{E , 2} , {n}_{C , 2}} - {t}_{{n}_{E , 1} , {n}_{C , 1}} ) + {t}_{{n}_{{E}^{, n} C}}}$$

$${θ}_{\frac{α}{2}} = {θ}_{0.5} - \frac{{Z}_{1 - \frac{a}{2} \sqrt{{T}_{N}}}}{\frac{\sqrt{{T}_{N} - {t}_{{n}_{E , 1} , {n}_{C , 1}}}}{\sqrt{{T}_{{N}_{new , 1}} - {t}_{{n}_{E , 1} , {n}_{C , 1}}}} ( \sqrt{{T}_{{N}_{new , 1}} - {t}_{{n}_{E , 2} , {n}_{C , 2}}} \sqrt{{T}_{{N}_{new , 2}} - {t}_{{n}_{E , 2} , {n}_{C , 2}}} + {t}_{{n}_{E , 2} , {n}_{C , 2}} - {t}_{{n}_{E , 1} , {n}_{C , 1}} ) + {t}_{{n}_{{E}^{, n} C}}}$$

$${θ}_{1 - \frac{α}{2}} = {θ}_{0.5} + \frac{{Z}_{1 - \frac{a}{2} \sqrt{{T}_{N}}}}{\frac{\sqrt{{T}_{N} - {t}_{{n}_{E , 1} , {n}_{C , 1}}}}{\sqrt{{T}_{{N}_{new , 1}} - {t}_{{n}_{E , 1} , {n}_{C , 1}}}} ( \sqrt{{T}_{{N}_{new , 1}} - {t}_{{n}_{E , 2} , {n}_{C , 2}}} \sqrt{{T}_{{N}_{new , 2}} - {t}_{{n}_{E , 2} , {n}_{C , 2}}} + {t}_{{n}_{E , 2} , {n}_{C , 2}} - {t}_{{n}_{E , 1} , {n}_{C , 1}} ) + {t}_{{n}_{{E}^{, n} C}}}$$

### Example 7

An important aspect of conducting interim analyses is the cost associated with preparation of the data for the data monitoring committee (DMC) meeting in terms of time and manpower involved. It is the main reason for the current monitoring to be occasional. The present invention has shown that the occasional monitoring only takes a snapshot of the data, hence it is subject to more uncertainty. In contrast, the continuous monitoring utilizes the up-to-date data at each patient entry, reveals the trend rather than a single time-point snapshot. The concern of cost is being much mitigated by implementing the DAD/DDM tool for the DMC to use.

### Feasibility of DDM

The DDM process requires continuously monitoring the on-going data. This involves continuous unblinding the data and calculating the monitoring statistics. It was unfeasible to handle it by an Independent Statistical Group (ISG). With the development of technologies nowadays, nearly all trials are managed by an Electronic Data Capture (EDC) system and the treatment assignment is processed by using the Interactive Responding Technology (IRT) or Interactive Web-Responding System (IWRS). Many off-shelf systems have EDC and IWRS integrated. The unblinding and calculation tasks can be carried out within an integrated EDC/IWRS system. This will avoid human-involved unblinding and preserve the data integrity. Although the technical details of machine-assisted DDM is not the focus of this article, it is worth noting that the DDM is feasible by utilizing the existing technologies.

### Data-Guided Analysis

With the DDM, the data-guided analysis can be started as early as practically possible. This can be built into a DDM engine so that the analysis can be performed automatically. The automation mechanism is in fact utilizing the “Machine Learning (M.L)” idea. The data-guided adaptation options, such as sample size re-estimation, dose selection, population enrichment, etc. can be viewed as applying Artificial Intelligence (A.I) technology to on-going clinical trials. Obviously, DDM with M.L and A.I can be applied to broader areas, such as the Real-World Evidence (RWE) and Pharmacovigilance (PV) for signal detection.

### Implementing the Dynamic Adaptive Designs

Increased flexibility associated with the DAD procedure improves efficiency of clinical trials. If used properly, it can help advance medical research, especially in rare diseases and trials in which per patient cost is expensive. However, the implementation of the procedure requires careful discussions. Measures to control and reduce the potential of operational bias can be critical. Such measures can be more effective and assuring if the specifics of potential biases can be identified and targeted. For practicality and feasibility, the procedures for implementing the adaptive sequential designs is well established. At the planned interim analysis, a Data Monitoring Committee (DMC) would receive the summary results from independent statisticians and hold a meeting for discussion. Although multiple sample size modifications are theoretically possible (e.g., see Cui, Hung, Wang, 1999; Gao, Ware, Mehta, 2008), it is usually not done more than once. Protocol amendments are usually made to reflect the DMC recommended changes. However, the DMC can hold unscheduled meetings for safety evaluations (in some diseases, efficacy endpoints are also safety endpoints). The current setting of the DMC, with minor modifications, can be used to implement the dynamic adaptive designs. The main difference is that, with the dynamic adaptive design, there may not be scheduled DMC efficacy review meetings. Trend analysis can be done by independent statisticians as the data accumulates (this can be facilitated with an electronic data capturing (EDC) system from which data can be constantly downloaded), but the results do not need to be constantly shared with the DMC members (However, if necessary and permissible by regulatory authorities, the trend analysis results may be communicated to DMC members through some secure web site, accessible through mobile devices, without needing any formal DMC meetings), and the DMC may be notified when a formal DMC review and decision is deemed necessary. Because most trials do amend the protocol multiple times, more than one amendment on sample size modification are not necessarily an increased burden, considering the benefit of improved efficiency. However, such decisions are to be made by the sponsors.

### DAD and DMC

The present invention introduced the Dynamic Data Monitoring concept and demonstrated its advantages for improving the trial efficiency. The advanced technology makes it possible to be implemented in future clinical trials.

A direct application of DDM may be for Data Monitoring Committee (DMC), which is formed for most of Phase II-III clinical trials. The DMC usually meets every 3 or 6 months depending on specific study. For example, for an oncology trial with new regimen, the DMC may want to meet more frequent than a trial for non-life threating disease. The committee may want to meet more frequent at early stage of the trial to understand the safety profile sooner. The current practice for DMC involves three parties: Sponsor, Independent Statistical Group (ISG) and DMC. The sponsor's responsibility is to conduct and manage the on-going study. The ISG prepares blinded and unblinded data packages: tables, listing and figures (TLFs) based on scheduled data cut (usually a month before the DMC meeting). The preparation work usually takes about 3-6 months. The DMC members receive the data packages a week before the DMC meeting and will review it during the meeting.

There are some issues in current DMC practice. First, the data package presented is only a snapshot of the data. The DMC couldn't see the trend of treatment effect (efficacy or safety) as data accumulated. Recommendation based on the snapshot of data may differ from that based on a continuous trace of data as illustrated in the following plots. In part a, DMC may recommend both trials to continue at interim 1 and 2, whereas in part b, the DMC may recommend terminating trial 2 due to its negative trend.

The current DMC process also has a logistic issue. It takes about 3-6 months for ISG to prepare data package for DMC. For a blinded study, the unblinding is usually handled by ISG. Although it is assumed that the data integrity will be preserved at ISG level, it is not 100% warranted by a human process. EDC/IWRS systems facilitated with DDM will have advantages of key safety and efficacy data to be monitored by DMC directly in real time.

### Incorporating Sample Size Reduction to Improving Efficiency

Theoretically, sample size reduction is valid with both the dynamic adaptive design and the adaptive sequential designs (e.g. Cui, Hung, wang, 1999, Gao, Ware, Mehta, 2008). Our simulations on both ASD and DAD show that incorporating sample size reduction can improve efficiency. However, due to concerns about “operating bias”, in current practice, sample size modification usually means sample size increase.

### Comparison of Non-Fixed Sample Designs

Besides ASD, there are other non-fixed sample designs. Lan el al (1993) proposed a procedure in which the data is continuous monitored. The trial can be stopped early if the actual effect size is larger than the assumed one, but the procedure does not include SSR. Fisher's “Self-designing clinical trials” (Fisher (1998), Shen, Fisher (1999)), is a flexible design that does not fix the sample size in the initial design but let the observations from “interim looks” guide the determination of the final sample size. It also allows for multiple sample size corrections through “variance spending”. Group sequential design, ASD, the procedure by Lan el al (1993) are all multiple testing procedures in which a hypothesis test is conducted at each interim analysis, and thus some alpha must be spent each time to control type I error (e.g. Lan, DeMets, 1983, Proschan et al (1993)). On the other hand, Fisher's self-designing trial is not a multiple testing procedure, because no hypothesis testing is conducted at the “interim looks”, and hence no alpha spending is necessary to control type I error, as explained in Shen, Fisher (1999): “A significant distinction between our method and the classical group sequential methods is that we will not test for the positive treatment effect in the interim looks.” The type I error control is achieved using a weighted statistic. So, the self-designing trials does possess the majority of the aforementioned “added flexibilities”, however, it is not based on multi-timepoint analysis and it does not provide unbiased point estimate, nor confidence interval. The following table summarizes the similarities and differences among the methods.

### Example 8

A Randomized, double-blind, placebo-controlled, exploratory Phase IIa study was conducted to assess the safety and efficacy of an orally administered drug candidate. The study failed to demonstrate efficacy. The DDM procedure was applied on the study database, displaying the trend of the whole study.

The relevant plots include Estimation of Primary Endpoint with 95% Confidence Interval, Wald Statistics (see FIG. 22), Score Statistics, Conditional Power and Sample Size Ratio (New sample size/Planned sample size). The plots of Score Statistics, Conditional Power and Sample size are stable and close to zero (no plot is shown here). As the plots of different doses (all dose, low dose, and high dose) vs Placebo exhibits similar trend and pattern, only all dose vs placebo is typically shown in FIG. 22 here. The plots started from at least two patients in each group for the reason of standard deviation estimation. The x-axis is time of patients' completion of study. The plots were updated after every patient completing study.

1): All dose vs Placebo

2): Low dose vs Placebo (1000 mg)

3): High dose vs Placebo (2000 mg)

### Example 9

A multi-center, double-blinded, placebo-controlled, 4-arm, Phase II Trial on a drug candidate for treatment of Nocturia has demonstrated safety and efficacy, and DDM procedure was applied on the study database, displaying the trend of the whole study.

The relevant plots include Estimation of Primary Endpoint with 95% Confidence Interval, Wald Statistics (FIG. 23A), Score Statistics, Conditional Power (FIG. 23B) and Sample Size Ratio (New sample size/Planned sample size) (FIG. 23C). As the plots of different doses (all dose, low dose, medium dose and high dose) vs Placebo exhibits similar trend and pattern, only all dose vs placebo is representatively shown here.

The plots start from at least two patients in each group for the reason of standard deviation estimation. The x-axis is time of patients' completion of study. The plots were updated after every patient completing study.

1: All dose vs Placebo
2: Low dose vs Placebo
3: Mid dose vs Placebo
4: High dose vs Placebo

### REFERENCES

1. Chandler, R. E., Scott, E. M., (2011). Statistical Methods for Trend Detection and Analysis in the Environmental Sciences. John Wiley & Sons, 2011
2. Chen Y H, DeMets D L, Lan K K. Increasing the sample size when the unblinded interim result is promising. Statistics in Medicine 2004; 23:1023-1038.
3. Cui, L., Hung, H. M., Wang, S. J. (1999). Modification of sample size in group sequential clinical trials. Biometrics 55:853-857.
4. Fisher, L. D. (1998). Self-designing clinical trials. Stat. Med. 17:1551-1562.
5. Gao P, Ware J H, Mehta C. (2008), Sample size re-estimation for adaptive sequential designs. Journal of Biopharmaceutical Statistics, 18: 1184-1196, 2008
6. Gao P, Liu L. Y, and Mehta C. (2013). Exact inference for adaptive group sequential designs. Statistics in Medicine. 32, 3991-4005
7. Gao P, Liu L. Y., and Mehta C. (2014) Adaptive Sequential Testing for Multiple Comparisons, Journal of Biopharmaceutical Statistics, 24:5, 1035-1058
8. Herson, J. and Wittes, J. The use of interim analysis for sample size adjustment, Drug Information Journal, 27, 753D760 (1993).
9. Jennison C, and Turnbull BW. (1997). Group sequential analysis incorporating covariance information. J. Amer. Statist. Assoc., 92, 1330-1441.
10. Lai, T. L., Xing, H. (2008). Statistical models and methods for financial markets. Springer.
11. Lan, K. K. G., DeMets, D. L. (1983). Discrete sequential boundaries for clinical trials. Biometrika 70:659-663.
12. Lan, K. K. G. and Wittes, J. (1988). The B-value: A tool for monitoring data. Biometrics 44, 579-585.
13. Lan, K. K. G. and Wittes, J. ‘The B-value: a tool for monitoring data’, Biometrics, 44, 579-585 (1988).
14. Lan, K. K. G. and DeMets, D. L. ‘Changing frequency of interim analysis in sequential monitoring’, Biometrics, 45, 1017-1020 (1989).
15. Lan, K. K. G. and Zucker, D. M. ‘Sequential monitoring of clinical trials: the role of information and Brownian motion’, Statistics in Medicine, 12, 753-765 (1993).
16. Lan, K. K. G., Rosenberger, W. F. and Lachin, J. M. Use of spending functions for occasional or continuous monitoring of data in clinical trials, Statistics in Medicine, 12, 2219-2231 (1993).
17. Tsiatis, A. ‘Repeated significance testing for a general class of statistics used in censored survival analysis’, Journal of the American Statistical Association, 77, 855-861 (1982).
18. Lan, K. K. G. and DeMets, D. L. ‘Group sequential procedures: calendar time versus information time’, Statistics in Medicine, 8, 1191-1198 (1989).
19. Lan, K. K. G. and Demets, D. L. Changing frequency of interim analysis in sequential monitoring, Biometrics, 45, 1017-1020 (1989).
20. Lan, K. K. G. and Lachin, J. M. ‘Implementation of group sequential logrank tests in a maximum duration trial’, Biometrics. 46, 657-671 (1990).
21. Mehta, C., Gao, P., Bhatt, D. L., Harrington, R. A., Skerjanec, S., and Ware J. H., (2009)
Optimizing Trial Design: Sequential, Adaptive, and Enrichment Strategies, Circulation, Journal of the American Heart Association, 119; 597-605 (including online supplement made apart thereof).
22. Mehta, C.R., and Ping Gao, P. (2011) Population Enrichment Designs: Case Study of a Large Multinational Trial, Journal of Biopharmaceutical Statistics, 21:4 831-845.
23. Müller, H. H. and Scháfer, H. (2001). Adaptive group sequential designs for clinical trials: combining the advantages of adaptive and of classical group sequential approaches. Biometrics 57, 886-891.
24. NASA standard trend analysis techniques (1988). https://elibrary.gsfc.nasa.gov/_assets/doclibBidder/tech_docs/29.%20NASA_STD_8070.5% 20-%20Copy.pdf
25. O'Brien, P. C. and Fleming, T. R. (1979). A multiple testing procedure for clinical trials. Biometrics 35, 549-556.
26. Pocock, S. J., (1977), Group sequential methods in the design and analysis of clinical trials. Biometrika, 64, 191-199.
27. Pocock, S. J. (1982). Interim analyses for randomized clinical trials: The group sequential approach. Biometrics 38, (1):153-62.
28. Proschan, M. A. and Hunsberger, S. A. (1995). Designed extension of studies based on conditional power. Biometrics, 51(4):1315-24.
29. Shih, W. J. (1992). Sample size reestimation in clinical trials. In Biopharmaceutical Sequential Statistical Applications, K. Peace (ed), 285-301. New York: Marcel Dekker.
30. Shih, W. J. Commentary: Sample size re-estimation—Journey for a decade. Statistics in Medicine 2001; 20:515-518.
31. Shih, W. J. Commentary: Group sequential, sample size re-estimation and two-stage adaptive designs in clinical trials: a comparison. Statistics in Medicine 2006; 25:933-941.
32. Shih W J. Plan to be flexible: a commentary on adaptive designs. Biom J; 2006; 48(4):656-9; discussion 660-2.
33. Shih, W. J. “Sample Size Reestimation in Clinical Trials” in Biopharmaceutical Sequential Statistical Analysis. Editor: K. Peace. Marcel-Dekker Inc., New York, 1992, pp. 285-301.
34. K. K. Gordon Lan John M. Lachin Oliver Bautista Over-ruling a group sequential boundary—a stopping rule versus a guideline. Statistics in Medicine, Volume 22, Issue 21
35. Wittes, J. and Brittain, E. (1990). The role of internal pilot studies in increasing the efficiency of clinical trials. Statistics in Medicine 9, 65-72.
36. Xi D, Gallo P and Ohlssen D. (2017). On the optimal timing of futility interim analyses. Statistics in Biopharmaceutical Research, 9:3, 293-301.

What is claimed is:

1. A graphical user interface-based system for dynamically monitoring and evaluating an on-going clinical trial associated with a disease or condition, said system comprising:
(1) a data collection system that dynamically collects blinded data from said on-going clinical trial in real time,
(2) an unblinding system, operable with said data collection system, that automatically unblinds said blinded data into unblinded data,
(3) an engine that continuously calculates statistical quantities, threshold values and success and failure boundaries based on said unblinded data and exports to a graphical user interface (GUI), and
(4) an outputting unit that dynamically outputs to said GUI an evaluation result indicating one of the following:
said on-going clinical trial is promising; and
said on-going clinical trial is hopeless;
wherein said GUI comprises a menu allowing a user to select from a group of statistical quantities comprising maximum trend ratio (mTR), sample size ratio (SSR), and mean trend ratio, to be displayed on said GUI.

2. The system of claim 1, wherein said group of statistical quantities further comprises Score statistics, point estimate (\hat{θ}) and its 95% confidence interval, Wald statistics (Z(t)), and conditional power (CP(θ,t,C|u)) calculated by
$$C P ( θ , N , C \vert u ) = P ( \frac{{s}_{N}}{\sqrt{{I}_{N}}} \geq C \vert {S}_{{n}_{E} , {n}_{C}} = u ) = 1 - Φ ( \frac{C \sqrt{{I}_{N}} - u - θ ( {I}_{N} - {i}_{{n}_{E} , {n}_{C}} )}{\sqrt{{I}_{N} - {i}_{{n}_{E} , {n}_{C}}}} ) ,$$
wherein Φ is the standard normal distribution function.

3. The system of claim 2, wherein said GUI reveals via a subsection thereof that said on-going clinical trial is promising, when one or more of the following are met:
(1) value of the Score statistics is constantly trending up or is constantly positive along information time,
(2) the slope of a plot of the Score statistics versus information time is positive,
(3) value of said mTR is in the range of (0.2, 0.4),
(4) value of said mean trend ratio is no less than 0.2, and
(5) said sample size ratio (SSR) is no more than 3.

4. The system of claim 3, wherein said GUI reveals via a subsection thereof that said on-going clinical trial is hopeless, when one or more of the following are met:
(1) value of said mTR is less than −0.3, and said point estimate is negative,
(2) said point estimate is observed to be negative for over 90 times (count each pair),
(3) value of said Score statistics is constantly trending down or is constantly negative along information time,
(4) the slope of a plot of said Score statistics versus information time is zero or near zero, and there is no or very limited chance for said Score statistics to cross said success boundary with a statistically significant level p<0.05, and
(5) said sample size ratio (SSR) is greater than 3.

5. The system of claim 4, wherein, when said on-going clinical trial is promising, said engine further conducts another evaluation of said on-going clinical trial and outputs to said GUI another result indicating whether a sample size adjustment is needed.

6. The system of claim 5, wherein said GUI reveals that no sample size adjustment is needed when said SSR is stabilized in the range of [0.6, 1.2].

7. The system of claim 6, wherein said GUI reveals that a sample size adjustment is needed when said SSR is stabilized and less than 0.6 or greater than 1.2.

8. The system of claim 1, wherein said data collection system is an Electronic Data Capture (EDC) System or Interactive Web Respond System (IWRS).

9. The system of claim 1, wherein said engine is a Dynamic Data Monitoring (DDM) engine.

10. The system of claim 1, wherein said desired conditional power is at least 90%.

11. A graphical user interface-based method of dynamically monitoring and evaluating an on-going clinical trial associated with a disease or condition, said method comprising:
(1) dynamically collecting blinded data by a data collection system from said on-going clinical trial,
(2) automatically unblinding said blinded data by an unblinding system operable with said data collection system into unblinded data,
(3) continuously calculating statistical quantities, threshold values, and success and failure boundaries by an engine based on said unblinded data, wherein said statistical quantities, threshold values, and success and failure boundaries are communicated to a graphical user interface (GUI), and
(4) dynamically outputting to said GUI an evaluation result indicating one of the following:
said on-going clinical trial is promising, and
said on-going clinical trial is hopeless,
wherein said GUI comprises a menu allowing a user to select from a group of statistical quantities comprising maximum trend ratio (mTR), sample size ratio (SSR), and mean trend ratio, to be displayed on said GUI.

12. The method of claim 11, wherein said group of statistical quantities further comprises Score statistics, point estimate (\hat{θ}) and its 95% confidence interval, Wald statistics (Z(t)), and conditional power (CP(θ, t, C|u)) calculated by
$$C P ( θ , N , C \vert u ) = P ( \frac{{s}_{N}}{\sqrt{{I}_{N}}} \geq C \vert {S}_{{n}_{E} , {n}_{C}} = u ) = 1 - Φ ( \frac{C \sqrt{{I}_{N}} - u - θ ( {I}_{N} - {i}_{{n}_{E} , {n}_{C}} )}{\sqrt{{I}_{N} - {i}_{{n}_{E} , {n}_{C}}}} ) ,$$
wherein Φ is the standard normal distribution function.

13. The method of claim 12, wherein said GUI reveals that said on-going clinical trial is promising, when one or more of the following are met:
(1) value of said mTR is in the range of (0.2, 0.4),
(2) value of said mean trend ratio is no less than 0.2,
(3) value of said Score statistics is constantly trending up or is constantly positive along information time,
(4) the slope of a plot of said Score statistics versus information time is positive, and
(5) said sample size ratio (SSR) is no more than 3.

14. The method of claim 12, wherein said GUI reveals that said on-going clinical trial is hopeless, when one or more of the following are met:
(1) value of said mTR is less than −0.3, and said point estimate is negative;
(2) said point estimate is observed to be negative for over 90 times (count each pair);
(3) value of said Score statistics is constantly trending down or is constantly negative along information time;
(4) the slope of a plot of said Score statistics versus information time is zero or nearly zero, and there is no or very limited chance for said Score statistics to cross said success boundary with a statistically significant level p<0.05; and
(5) said sample size ratio (SSR) is greater than 3.

15. The method of claim 13, wherein, when said on-going clinical trial is promising, said method further comprises conducting another evaluation of said on-going clinical trial and outputting to said GUI another result indicating whether a sample size adjustment is needed.

16. The method of claim 15, wherein said GUI reveals that no sample size adjustment is needed when said SSR is stabilized in the range of [0.6, 1.2].

17. The method of claim 15, wherein said GUI reveals that a sample size adjustment is needed when said SSR is stabilized and less than 0.6 or greater than 1.2.

18. The method of claim 1, wherein said data collection system is an Electronic Data Capture (EDC) System, or Interactive Web Respond System (IWRS).

19. The method of claim 11, wherein said engine is a Dynamic Data Monitoring (DDM) engine.

20. The method of claim 11, wherein said desired conditional power is at least 90%.
